(19) United States
(12) Patent Application Publication
(10) Pub. No.: US 2026/0297546 A1
(43) Pub. Date: Oct. 1, 2026

(54) ENGINEERED TYPE III RNA-TARGETING CRISPR EFFECTORS

(71) Applicant: THE REGENTS OF THE UNIVERSITY OF CALIFORNIA, Oakland, CA (US)

(72) Inventor: Daniel Brogan, La Jolla, CA (US)

(21) Appl. No.: 19/478,614

(22) PCT Filed: Apr. 26, 2024
(86) PCT No.: PCT/US2024/026605

§ 371 Date: Oct. 24, 2025

Related U.S. Application Data
(60) Provisional application No. 63/462,195, filed on Apr. 26, 2023.

(51) Int. Cl. C12N 9/22; C12N 15/11; C12N 15/86; C12N 15/90

(52) U.S. Cl. CPC C12N 9/224; C12N 15/11; C12N 15/86; C12N 15/907; C12N 2310/20; C12N 2750/14143

(57) ABSTRACT

This disclosure provides a synthetic or engineered protein or polypeptide comprising a Cas7.1 peptide and a Casl or Casl 1 peptide, the synthetic or engineered protein or polypeptide having RNA-targeting function. This disclosure further provides CRISPR systems including the polypeptide or protein and methods of use of the CRISPR systems.

### CROSS-REFERENCE TO RELATED APPLICATIONS

This application claims priority under 35 U.S.C. § 119(e) to U.S. Provisional Application No. 63/462,195, filed Apr. 26, 2023, the content of which is hereby incorporated by reference in its entirety.

### STATEMENT OF GOVERNMENT SUPPORT

This invention was made with government support under NIH/NIAID Grant Numbers R01GM132825-04, R21RAI149161A, and DP2AI152071 awarded by the National Institutes of Health/National Institute of Allergy and Infectious Diseases. The government has certain rights in the invention.

### BACKGROUND OF THE DISCLOSURE

The continuous evolution of CRISPR-Cas systems has resulted in the discovery and engineering of revolutionary genome engineering technologies. Recently, the type III-E CRISPR-Cas system was discovered (1) and characterized (2, 3) and has changed how the understanding of Cas protein evolution. Type III-E effectors are categorized as class 1 CRISPR-Cas systems, which typically utilize a multiprotein complex to accomplish DNA- and/or RNA-targeting (1). However, type III-E Cas effectors accomplish programmable RNA-targeting by employing a single, large protein composed of four Cas7-like domains, one Cas11 domain, and a large insertion (INS) domain, hence being named Cas7-11 (4, 5) (or gRAMP for giant Repeat Associated Mysterious Proteid (6)). Recent studies have demonstrated that type III-E effectors are capable of programmable RNA knockdown in prokaryotic and eukaryotic cells, and do not exhibit RNA collateral cleavage activity that consistently limits applications of CRISPR-Cas13 systems (7-10). Using prokaryotic expression, it was demonstrated that Cas7-11 target binding activates a downstream caspase activity that results in cell dormancy, or death, and appears to serve as the primary immune response for type III-E CRISPR-Cas systems (6, 11, 12). Therefore when type III-E effectors are expressed independently of their protease signaling pathway, they operate as highly specific ribonucleases and their unique architecture provides a pathway for further engineering.

It is important to consider effector size when engineering CRISPR-Cas systems, and efforts have been made to generate compact effectors for almost all therapeutically relevant CRISPR-Cas systems. Usually, Class I CRISPR-Cas systems are extremely challenging to encode in therapeutic vectors since they require co-expression of multiple independent proteins (13), which highlights the advantage of using Cas7-11 instead. However, type III-E effectors are the largest single-effector Cas protein subtype discovered to date (2, 3), which is a limitation for therapeutic applications, such as AAV delivery. For RNA-targeting CRISPR-Cas systems, Cas13 effectors, such as the compact Cas13d subtype (14, 15), are more feasible for packaging, however severely limited by collateral cleavage of ssRNA (7). Recently, a small Cas7-11 (Cas7-11S) protein was engineered through deletion of the INS domain and replacement with a GS-linker (4).

### SUMMARY OF THE DISCLOSURE

Provided herein are several compact effectors for programmable RNA-targeting in mammalian cells. Applicant identified a novel composition of a type III-E-like effector composed of Cas7-like and a Cas1-like domain, that can be engineered into an active chimeric RNA-targeting Cas effector and presents a new function of Cas1 in RNA-targeting. Furthermore, Applicant demonstrate a unique modularity of type III-E effectors by methodically substituting domains between orthogonal type III-E proteins to engineer compact synthetic Cas effectors. Cas7-S represents a new understanding of type III-E architecture and modularity, and provides a platform for engineering genome engineering technologies from the blueprint of nature.

Provided herein are naturally occurring CRISPR proteins with missing elements engineered with workable parts from other CRISPR proteins to create a synthetic protein by combining the crRNA recognition domain from other Type III-E CRISPR effectors used to program the HyCas system to target select RNA molecules. In particular, the synthetic RNA-targeting Cas effectors can be engineered to be compact in size. The HyCas system combines essential features of Cas7-11 with a novel, uncharacterized Cas7-1 protein to generate a synthetic programmable nuclease with RNA-targeting capabilities. The HyCas systems can programmably target and degrade RNA molecules. Also included here is a method for generating synthetic fusion proteins of the Type III-E CRISPR effector class. The Cas7-1 protein has not been characterized, nor shown to be functional and through the fusion with the crRNA processing domain of Cas7-11, the Cas7-1 protein is capable of RNA-targeting. Even more unique is no known CRISPR protein has repurposed a Cas1 nuclease domain for targeted degradation making this technology a novel formation and function for the Cas1 nuclease domain.

In one aspect, this disclosure provides a synthetic or engineered protein comprising a Cas7-11 peptide and a Cas7-1 peptide with RNA-targeting function. Also provided is a synthetic protein, as described herein as well as equivalents thereof, that provide the protein with RNA-targeting function. In one aspect, the synthetic protein comprises a crRNA recognition domain from an uncharacterized Cas7-1 peptide.

Further provided are CRISPR systems comprising the synthetic protein as described herein and for example, as described in any one of the examples and figures.

Also provided are compositions comprising the synthetic proteins and/or CRISPR systems as described herein and a carrier. In one aspect, the carrier is a pharmaceutically acceptable carrier.

The synthetic proteins, CRISPR systems and compositions are useful to target and cleave an RNA molecule, by contacting the RNA with the synthetic proteins and/or CRISPR systems.

### BRIEF DESCRIPTION OF THE DRAWINGS

FIGS. 1A-1G: Cas7-1 sequence and predictive structure in relation to type III-E effectors. (FIG. 1A) Schematic representation of a typical Cas7-11 architecture and the architecture of Cas7-1. (FIG. 1B) Alignment of catalytic residue in the Cas7.3 domain of Cas7-1 against published Cas7-11 proteins (3). (FIG. 1C) Alignment of zinc finger residues in the Cas7.3 and Cas7.4 domains of Cas7-1 and Cas7-11 proteins. Amino Acid (AA) position represents position in Cas7-1 protein sequence. (FIG. 1D) Structural alignment of Cas7.3 zinc finger residues of Cas7-1 with aligned residues of DiCas7-11. (FIG. 1E) Structural alignment of Cas7.4 zinc finger residues of Cas7-1 with aligned residues of DiCas7-11. (FIG. 1F) Alignment of Cas1 domain of Cas7-1 to MMB1 RT-Cas1 fusion protein (NCBI Ascension here). An additional alignment of Cas7-1 and MMB-1RT-Cas1 is in FIG. 15. Highlighted sections represent catalytic domains of Cas1, where E847 of Cas7-1 aligns to E870 of MMB1 RT-Cas1. (FIG. 1G) Predicted structure of Cas7-1 obtained from AlphaFold 2. The sequences in FIG. 1 are the following: SEQ ID NO: 1 (Cas7-1), SEQ ID NO: 2 (CbfCas7-11), SEQ ID NO: 3 (CjcCas7-11), SEQ ID NO: 4 (CmaCas7-11), SEQ ID NO: 5 (CsbCas7-11), SEQ ID NO: 6 (DiCas7-11), SEQ ID NO: 7 (DpbaCas7-11), SEQ ID NO: 8 (DsbaCas7-11), SEQ ID NO: 9 (FmCas7-11), SEQ ID NO: 10 (GwCas7-11), SEQ ID NO: 11 (HreCas7-11), SEQ ID NO: 12 (HsmCas7-11), SEQ ID NO: 13 (HvmCas7-11), SEQ ID NO: 14 (HvsCas7-11), SEQ ID NO: 15 (OmCas7-11), SEQ ID NO: 16 (SmCas7-11), SEQ ID NO: 17 (SstCas7-11), SEQ ID NO: 18 (SybCas7-11).

FIGS. 2A-2D: Cas7-S construction and analysis of initial variants. (FIG. 2A) Schematic representing the construction of Cas7-S10 and a depiction of RNA degradation by Cas7-S10. Red lines represent domains from DiCas7-11 and blue lines represent domains from Cas7-1. (FIG. 2B) qPCR analysis of EGFP targeted knockdown with Cas7-S variants that have a C-terminal orientation of the Cas1 domain and Cas7-S variants that orient the Cas11 or Cas1 domain internally in the typical Cas7-11 architecture. (FIG. 2C) Predicted structure and partial amino acid sequence of Cas1 domain with highlighted areas representing truncation of the domain. (FIG. 2D) qPCR analysis quantifying EGFP knockdown by Cas7-S variants with different truncations of Cas1. In all qPCR plots, significance is calculated and determined using unpaired t-test between crRNA^{EGFP} and crRNA^{NT}. Error bars represent standard error of the mean (sem) (n=4).

FIGS. 3A-3H: Biochemical assessment of Cas7-S cleavage activity. (FIG. 3A) qPCR analysis of EGFP knockdown comparing 30 nt vs 22 nt spacers for DiCas7-11 and Cas7-S10. Significance is calculated and determined using unpaired t-test between crRNA^{EGFP} and crRNA^{NT}. Error bars represent sem (n=3). (FIG. 3B) Schematic depicting the crRNAs used for assessment of in vitro cleavage activity and depiction of cleavage and expected cleavage products. Arrowheads represent cut sites of crRNA-5. (FIG. 3C) Experiment tiling six 22 nt crRNAs across the 40 nt ssRNA target to obtain stepwise cleavage products. (FIG. 3D) Assessment of cleavage pattern and catalytic inactivation of DiCas7-11, Cas7-S10, and Cas7-S12 through site specific mutagenesis (D429A/D654A). (FIG. 3E) Schematic representation of different crRNA processing outcomes. (1) “single-guide” crRNA processing. (2) native array crRNA processing. (3) modified array crRNA processing. (FIG. 3F-FIG. 3G) crRNA array processing activity by DiCas7-11 (WT) and ΔINS mutant, Cas7-S12. Array structures represented by schematics above each lane. crRNA processing products and approximate location on gel depicted by schematics on the side of gel. (FIG. 3H) Comparison cleavage assay between WT DiCas7-11, Cas7-S10, and Cas7-S12 (ΔINS) with varying array structures. Arrows represent cleavage by Cas7.2 only. cleavage products by Cas7.3 or from both Cas7.2/Cas7.3 active sites cleaving the target, 20 nt processed DR fragments, and 15 nt mature direct repeats (DR) (3′ only).

FIGS. 4A-4E: Generation and validation of compact Cas7-S effectors. (FIG. 4A) Schematic depicting workflow for analysis of Cas7-S library with refined ODS design method. (FIG. 4B) qPCR analysis of EGFP knockdown with all 36 compact Cas7-S effectors. Data is broken into two groups HvsCas7.1-based (S17-S34) and DiCas7.1-based (S35-S52) Cas7-S effectors. Dashed lines represents average knockdown by DiCas7-11 (WT). Significance is calculated and determined using unpaired t-test between crRNA^{EGFP} and crRNA^{NT}. WT=wild type DiCas7-11. Error bars represent sem (n=4). (FIG. 4C) Flow cytometry analysis of select Cas7-S effectors demonstrating effect of RNA knockdown on translation of EGFP and mCherry. Data depicts population change in High or Low GFP, or mCherry, expression groups (see methods for gating). Error bars represent sem (n=4). (FIG. 4D) RNA knockdown of KRAS gene with DiCas7-11 and select Cas7-S effectors analyzed using qPCR. Error bars represent sem (n=3). (FIG. 4E) SENSR assay determining ssRNA collateral activity of Cas7-S effectors depicted by background corrected fluorescence levels. RfxCas13d was used as a positive control. Error bars represent sem (n=3). RFU=relative fluorescence unit.

FIGS. 5A-5E: Sequence and structure prediction of Cas7-1. (FIG. 5A) HHpred domain assignment results. Domains are segmented based on alignments to most likely results. Results are depicted from most likely to least likely. (FIG. 5B) AlphaFold predicted structure and heat map representation of Cas7-1. (FIG. 5C) Spatial alignment of Csm3 over Cas7-1. (FIG. 5D) Spatial alignment of Cas1 over Cas7-1. (FIG. 5E) Predicted domains from HHPred analysis.

FIGS. 6A-6D: Cas7-1 predicted DR processing and cleavage assessment. (FIG. 6A) Predicted fold of predicted 38 nt Cas7-1 direct repeat sequence DRX. (FIG. 6B) Predicted fold of predicted 38 nt Cas7-1 direct repeat sequence DRY. F represents 5′ orientation and R represents 3′ orientation of DR related to the spacer. (FIG. 6C) Predicted fold of known 35 nt DR for DiCas7-11. (FIG. 6D) Gel electrophoresis assessment of crRNA processing and targeted cleavage with predicted DRs of Cas7-1 compared with DiCas7-11. Arrows indicate cleavage products from Cas7.2 domain, cleavage products from Cas7.3 and/or Cas7.2/Cas7.3 domain(s), and 20 nt product from DR processing. The sequences in FIGS. 6A, 6B, and 6C are also included in Table 5.

FIG. 7: Domain breakdown of initial Cas7-S variants. Original proteins to build the initial Cas7-S variants are DiCas7-11 and Cas7-1. Domains in the Cas7-S variants originate from DiCas7-11 and/or Cas-7-1 proteins. Domains are not scaled.

FIGS. 8A-8D: Design and assessment of Cas7-S effectors. (FIG. 8A) Schematic representation of 2-effector design strategy with Cas7-S10 (left) and Cas7-S14 (right) depicted. Top effector is DiCas7-11 and bottom effector is Cas7-1. (FIG. 8B) Schematic representation of 3-effector design strategy with Cas7-S41 (right) and Cas7-S47 (left) depicted. Top effector is DiCas7-11, bottom left is Cas7-1, and bottom right is HvsCas7-11. (FIG. 8C) Size comparison between type III-E effectors and the various Cas7-S effectors generated in this study. (FIG. 8D) Heat map representation of qPCR data from Cas7-S17-52 analysis. The x- and y-axes show the protein of origin for select domains in the Cas7-S effector. For example, S35 (Cas7-S35) includes a DiCas7.1 (Cas7.1 domain from DiCas7-11), DiCas11 (Cas11 domain from DiCas7-11), HvsCas7.2 (Cas7.2 domain from HvsCas7-11), and HvsCas7.3 (Cas7.3 domain from HvsCas7-11) The more intense the saturation the stronger the knockdown level. Data shown in the heatmap are 4 replicates in each cell.

FIG. 9: Alignment of 29AA N-terminus of Cas1 domain to RT-Cas1 fusion proteins. The Cas7-1 Cas1 N-terminal linker domain (SEQ ID NO: 1) aligns well with multiple N-terminal regions of RT-Cas1 fusion proteins. NCBI ascension identities listed for each RT-Cas1 protein: MBU0568284.1, MCR4321456.1, MBI4691638.1, KHE91657.1, and MCF6147510.1. Only a portion of the RT-Cas1 fusion proteins are included in the alignment. The portion of the of the Cas1 N-terminal linker domain that aligns well with the RT-Cas1 fusion proteins is underlined.

FIGS. 10A-10B: Biochemical assessment of Cas7-S10. (FIG. 10A) Examination of the influence of spacer length on cleavage activity of Cas7-S10. Arrows indicate cleavage products from Cas7.2 domain, cleavage products from Cas7.3 and/or Cas7.2/Cas7.3 domain(s), and/or 20 nt product from DR processing. The asterisk next to the 20 nt spacer Cas7.2 cleavage indicates a cleavage product and processed crRNA. (FIG. 10B) Assessment of cleavage activity for Cas7-S10 over time (1.5 hr, 3 hr, 6 hr, and 12 hr).

FIG. 11: Domain breakdown of compact Cas7-S variants. Original proteins to build the initial Cas7-S variants are DiCas7-11, HvsCas7-11, and Cas7-1. Domains of the Cas7-S variants can include domains from DiCas7-11 and/or HvsCas7-11 and/or Cas7-1. While not included in the figures, HvsCas7-11 also includes a CTE domain. The CTE domain in HvsCas7-11 is included on the schematic in FIG. 8B. The placement of the HvsCas7-11 CTE domain is the same on HvsCas7-11 as the placement of the DiCas7-11 CTE domain is on DiCas7-11 (shown in FIG. 11).

FIGS. 12A-12C: Cas7-S Targeted RNA knockdown comparison against RfxCas13d and DiCas7-11. (FIG. 12A) qPCR analysis of RNA knockdown targeting GFP in HEK293T cells. (FIG. 12B) GFP knockdown of each effector relative to RfxCas13d. (FIG. 12C) GFP knockdown of each effector relative to DiCas7-11. Significance is calculated and determined using unpaired t-test between crRNA^{EGFP} and crRNA^{NT}. Error bars represent sem (n=3).

FIGS. 13A-13D: Representative gating method for flow cytometry analysis of EGFP knockdown. (FIG. 13A) Area of cells with polygon identifying selected cells. (FIG. 13B) Further refinement of selected cells—FSC width vs area. (FIG. 13C) Further refinement of selected cells-SSC width vs area. (FIG. 13D) Defined range of high EGFP population.

FIG. 14: Breakdown of domains for Type III-E CRISPR-Cas effectors based on clustal omega sequence alignment. Numbers in individual cells represent the length (amino acid) of the sequences. The number of amino acids in each domain added together is the length of the effector. As seen in FIG. 1A, for example, the Cas7-11 effectors domain layout and the Cas7-1 effector domain layout follows the exemplary architecture of the effectors. For example, in the effectors the INS domain is within the Cas7.4 domain. In FIG. 14 however, only the full length of the Cas7.4 domain is given (i.e. Cas7-1 (com)), not indicating placement of INS within the domain.

FIG. 15: The alignment of the Cas7-1 Cas1 domain (SEQ ID NO: 1 amino acids 791-933, as seen in FIG. 1A) and MMB-1RT-Cas1 (Reverse Transcriptase-Cas1) fusion protein commonly associated with type III systems is shown.

FIG. 16: GFP RNA-knockdown by SynCas.v54 (also referred to herein as Cas7-S54). The graph above depicts qPCR analysis of programmable GFP knockdown by SynCas.v54 following transfection in HEK293 cells. The different crRNA types depict different crRNA array structures: DX1=2 spacers, DX2=3 spacers, and DX3=4 spacers, DF2=1 spacer, CG1=1 spacer with two direct repeats.

FIG. 17: Domain boundaries of the Cas7-S synthetic proteins. The number represents the position of the first and final amino acid for each domain. N/A indicates the domain is not present in the synthetic protein. Cas7.4-1 and Cas7.4-2 are both part of the same Cas7.4 domain, but indicates the placement of the INS domain within the Cas7.4 domain. For Cas7-S12, there are two embodiments. The first being shown in FIG. 17, and the second disclosed in Table 2.

### DETAILED DESCRIPTION

### Definitions

The following definitions are intended to support and describe the embodiments and aspects of this disclosure, including as described in the attached Appendices, incorporated herein by reference.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein, the singular forms “a”, “an”, and “the” include plural referents unless the context clearly dictates otherwise. Thus, for example, reference to “a cell” includes a plurality of cells.

As will be understood by one skilled in the art, for any and all purposes, all ranges disclosed herein also encompass any and all possible subranges and combinations of subranges thereof. Furthermore, as will be understood by one skilled in the art, a range includes each individual member.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series; Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail ed. (2004) Gene Silencing by RNA Interference: Technology and Application (CRC Press).

As used herein, the term “CRISPR” (Clustered Regularly Interspaced Short Palindromic Repeats) refers to a technique of sequence specific genetic manipulation relying on the clustered regularly interspaced short palindromic repeats pathway, which unlike RNA interference regulates gene expression at a transcriptional level. The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. (2014) Nature biotechnology; Graham, D., et al. (2015) Genome Biol. “Single guide RNA” or “sgRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct.

The term “Cas9” refers to a CRISPR associated endonuclease referred to by this name (UniProtKB G3ECR1 (CAS9_STRTR)) as well as deadCas-9 or dCas9, which lacks endonuclease activity.

Type III-E CRISPR effector intends an effector molecule use in the Type III-E CRISPR system. The Type III-E CRISPR system uses a single multidomain effector called Cas7-11 (also called gRAMP, for giant Repeat Associated Mysterious Proteid) to cleave RNA. Cas7-11 can associate with a caspase-like protease Csx29. See, for example, Yu, G. et al. (2022) “Structure and function of a bacterial type III-E CRISPR-Cas7-11 complex” Nat. Microbiol., Vol. 7:2078-2088, incorporated herein by reference.

Thus, as used herein, “CRISPR system” (also referred to herein as “CRISPR-Cas system”) refers collectively to transcripts and other elements involved in the expression of or directing the activity of CRISPR-associated (“Cas”) genes. This includes but is not limited to sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer”, “guide RNA” or “gRNA” in the context of an endogenous CRISPR system), or other sequences and transcripts from a CRISPR locus. One or more tracr mate sequences operably linked to a guide sequence (e.g., direct repeat-spacer-direct repeat) can also be referred to as “pre-crRNA” (pre-CRISPR RNA) before processing or crRNA (CRISPR RNA) after processing by a nuclease. The CRISPR system can include a Cas protein and crRNA.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced there from.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

The terms “equivalent” or “biological equivalent” are used interchangeably when referring to a particular molecule, biological, or cellular material and intend those having minimal homology while still maintaining desired structure or functionality.

As used herein, the term “functional” may be used to modify any molecule, biological, or cellular material to intend that it accomplishes a particular specified effect.

As used herein, the term “crRNA” refers to CRISPR RNA, known in the art to be used with the CRISPR-Cas system to facilitate targeting of the gene. As used herein, crRNAs include a direct repeat sequence and a sequence (also referred to as a spacer sequence) complementary to a target RNA sequence. The direct repeat (DR) associates with the Cas protein in the CRISPR-Cas system. crRNAs can be incorporated into plasmids which can include a promoter and terminator, for example a U6 promoter and terminator. crRNA is known in the art, including in Type III CRISPR-Cas systems (e.g. Kolesnik et al. (2021) Biochemistry (Mosc), 86(10): 1301-1314; Woodside et al. (2022) RNA, 28(8): 1074-1078, incorporated herein by reference). Where more than one crRNA is present in a construct, multiple spacers may be used to ensure gene targeting. The target specific sequences may be experimentally determined or found on one of many public databases, such as Addgene (www.addgene.org).

As used herein, the term “gRNA” refers to a guide RNA sequence, known in the art to be used with the CRISPR-Cas9 system to facilitate targeting of the gene. gRNAs typically comprise a promoter, gRNA scaffold, and a target specific sequence. Where more than one gRNA is present in a construct, spacers may be used to ensure gene targeting. The target specific sequences may be experimentally determined or found on one of many public databases, such as Addgene (www.addgene.org). gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 74-83).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include hU6 and mU6 promoter, CMV promoter, a T7 promoter, and EF-1α promoter. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency. Non-limiting examples of enhancers include an RSV enhancer or a CMV enhancer. Non-limiting exemplary promoter sequences are provided herein below:

CMV promoter:
ATACGCGTTGACATTGATTATTGACTAGTTATTAATAGTAATCAATTACGGGGTC
ATTAGTTCATAGCCCATATATGGAGTTCCGCGTTACATAACTTACGGTAAATGGC
CCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACC
ATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGGCACCA
AAATCAACGGGACTTTCCAAAATGTCGTAACAACTCCGCCCCATTGACGCAAAT
GGGCGGTAGGCGTGTACGGTGGGAGGTCTATATAAGCAGAGCTCGTTTAGTGAA
CCGTCAGATCGCCTGGAGACGCCATCCACGCTGTTTTGACCTCCATAGAAGACAC
CGGGACCGATCCAGCCTCCGGACTCTAGAGGATCGAACCCTT, or a biological
equivalent thereof.
U6 promoter:
GAGGGCCTATTTCCCATGATTCCTTCATATTTGCATATACGATACAAGGCTGTTA
GAGAGATAATTAGAATTAATTTGACTGTAAACACAAAGATATTAGTACAAAATA
CGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTT
ATATATCTTGTGGAAAGGACGAAACACC, or a biological equivalent thereof.
EF1a promoter:
CGTGAGGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTGGCCTC
TTTACGGGTTATGGCCCTTGCGTGCCTTGAATTACTTCCACGCCCCTGGCTGCAGT
ACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCG
CTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGC
CGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGGCG
GGTGAGTCACCCACACAAAGGAAAAGGGCCTTTCCGTCCTCAGCCGTCGCTTCAT
GTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCC
CACACTGAGTGGGTGGAGACTGAAGTTAGGCCAGCTTGGCACTTGATGTAATTCT
CCTTGGAATTTGCCCTTTTTGAGTTTGGATCTTGGTTCATTCTCAAGCCTCAGACA
GTGGTTCAAAGTTTTTTTCTTCCATTTCAGGTGTCGTGAG, or a biological equivalent
thereof.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “about” when used before a numerical designation, e.g., temperature, time, amount, concentration, and such other, including a range, indicates approximations which may vary by (+) or (−) 10%, 5% or 1%.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has, or is diagnosed of having, or is suspected of having, or is at risk of having a disease, such as a cancer or a hereditary disease such as sickle cell anemia or cystic fibrosis.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the composition or cell to provide the therapeutic benefit in vitro or in vivo by at least 10%, 25%, 40%, 60%, 80%, 90% or 95% as compared to control. Determination of these parameters is well within the skill of the art. These considerations, as well as effective formulations and administration procedures are well known in the art and are described in standard textbooks.

Administration or delivery in vivo can be effected in one dose, continuously or intermittently throughout the course of treatment. Methods of determining the most effective means and dosage of administration are well known to those of skill in the art and will vary with the composition used for therapy, the purpose of the therapy, the target cell, solid tumor or cancer being treated, and the subject being treated. Single or multiple administrations can be carried out with the dose level and pattern being selected by the treating physician.

The pharmaceutical compositions can be administered by inhalation, orally, intranasally, parenterally, injection, orally and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present disclosure, the compositions can also contain other pharmaceutically active compounds or a plurality of systems or cells of the disclosure.

More particularly, an agent of the present disclosure also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

While it is possible for the agent to be administered alone, it is preferable to present it as a pharmaceutical formulation comprising, or consisting essentially of, or consisting of at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present disclosure suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the agent, such carriers as are known in the art to be appropriate.

Formulations suitable for nasal administration or aerosol (directly into the lung), wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above, the formulations of this disclosure may include other agents conventional in the art having regard to the type of formulation in question, for example, those suitable for oral administration may include such further agents as sweeteners, thickeners and flavoring agents. It also is intended that the agents, compositions and methods of this disclosure be combined with other suitable compositions and therapies.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides, ribonucleotides, hybrid polynucleotides or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., WO 97/03211 and WO 96/39154. A polynucleotide may comprise one or more modified nucleotides, such as methylated nucleotides and nucleotide analogs or those as described herein. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “vector” refers to a construct which is capable of delivering, and, in some embodiments expressing, a polynucleotide in to a cell. Non-limiting examples of delivery vectors include viral vectors, nucleic acid expression vectors (such as a plasmid), naked DNA, and certain eukaryotic cells (e.g., producer cells). In some embodiments, nucleic acids described by the disclosure are delivered via a viral vector. Examples of viral vectors include retroviral vectors (e.g., Maloney murine leukemia virus, MML-V), adenoviral vectors (e.g., AD 100), lentiviral vectors (e.g., HIV and FIV-based vectors), and herpesvirus vectors (e.g., HSV, HSV-1, HSV-2), as described by Chira et al. (2015) Oncotarget, 6 (31): 30673-30703. In some embodiments, nucleic acids described by the disclosure are delivered by an adeno-associated virus (AAV) vector (e.g., a recombinant AAV (rAAV) vector).

The terms “non-naturally occurring” or “engineered” or “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridising to the reference sequence under highly stringent conditions. Generally, in order to maximize the hybridization rate, relatively low-stringency hybridization conditions are selected: about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the T m. In order to require at least about 70% nucleotide complementarity of hybridized sequences, moderately-stringent washing conditions are selected to be about 15 to 30° C. lower than the T m. Highly permissive (very low stringency) washing conditions may be as low as 50° C. below the T m, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. Exemplary highly stringent conditions comprise incubation in 50% formamide, 5×SSC, and 1% SDS at 42° C., or incubation in 5×SSC and 1% SDS at 65° C., with wash in 0.2×SSC and 0.1% SDS at 65° C.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “domain” or “protein domain” refers to a part of a protein sequence that may exist and function independently of the rest of the protein chain.

As described in aspects of this disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel (1990), GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al (1985), Cell, 41:521-530], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R—U5′ segment in LTR of HTLV-I (Mol. Cell. Biol. (1988), Vol. 8(1), p. 466-472); SV40 enhancer; and the intron sequence between exons 2 and 3 of rabbit β-globin (1981) (Proc. Natl. Acad. Sci. USA, Vol. 78(3), p. 1527-31). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., gene-editing system transcripts, proteins, enzymes, mutant forms thereof, fusion proteins thereof, etc.). With regards to promoters, mention is made of PCT publication WO 2011/028929, the content of which is incorporated by reference herein in their entirety.

Vectors can be designed for expression of gene-editing system transcripts (e.g. nucleic acid transcripts, proteins, or enzymes) in prokaryotic or eukaryotic cells. For example, gene-editing system transcripts can be expressed in bacterial cells such as Escherichia coli, insect cells (using baculovirus expression vectors), yeast cells, or mammalian cells. Suitable host cells are discussed further in Goeddel (1990), GENE EXPRESSION TECHNOLOGY: METHODS IN ENZYMOLOGY 185, Academic Press, San Diego, Calif. Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al. (1990), GENE EXPRESSION TECHNOLOGY: METHODS IN ENZYMOLOGY 185, Academic Press, San Diego, Calif. 60-89).

In some embodiments, a vector is a yeast expression vector. Examples of vectors for expression in yeast Saccharomyces cerevisae include pYepSec1 (Baldari, et al., (1987) EMBO J. 6:229-234), pMFa (Kuijan and Herskowitz, (1982) Cell 30:933-943), pJRY88 (Schultz et al., (1987) Gene 54:113-123), pYES2 (Invitrogen Corporation, San Diego, Calif.), and picZ (In Vitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., (1983) Mol. Cell. Biol. 3:2156-2165) and the pVL series (Lucklow and Summers, (1989) Virology 170:31-39).

In some embodiments, a vector is capable of driving expression of one or more sequences in mammalian cells using a mammalian expression vector. Examples of mammalian expression vectors include pCDM8 (Seed (1987) Nature 329:840) and pMT2PC (Kaufman, et al., (1987) EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al. (1989), MOLECULAR CLONING: A LABORATORY MANUAL. 2^{nd} ed., Cold Spring Harbor Laboratory, Cold Spring Harbor Laboratory Press, Cold Spring Harbor, N.Y.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., (1987) Genes Dev. 1:268-277), lymphoid-specific promoters (Calame and Eaton, (1988). Adv. Immunol. 43:235-275), in particular promoters of T cell receptors (Winoto and Baltimore, (1989) EMBO J. 8:729-733) and immunoglobulins (Baneiji, et al., (1983) Cell 33:729-740; Queen and Baltimore, (1983). Cell 33:741-748), neuron-specific promoters (e.g., the neurofilament promoter; Byrne and Ruddle, (1989). Proc. Natl. Acad. Sci. USA 86:5473-5477), pancreas-specific promoters (Edlund, et al., (1985), Science 230:912-916), and mammary gland-specific promoters (e.g., milk whey promoter; U.S. Pat. No. 4,873,316 and European Application Publication No. 264,166). Developmentally regulated promoters are also encompassed, e.g., the murine hox promoters (Kessel and Gruss, (1990). Science 249:374-379) and the α-fetoprotein promoter (Campes and Tilghman, (1989). Genes Dev. 3:537-546). With regards to these prokaryotic and eukaryotic vectors, mention is made of U.S. Pat. No. 6,750,059, the contents of which are incorporated by reference herein in their entirety. Tissue-specific regulatory elements are known in the art and in this regard, mention is made of U.S. Pat. No. 7,776,321, the contents of which are incorporated by reference herein in their entirety.

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, (1989), Handbook of Parvoviruses, Vol. 1, pp. 169-228, and Berns, (1990), Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, (1988), pp. 165-174 of Parvoviruses and Human Disease, J. R. Pattison, ed.; and Rose, (1974) Comprehensive Virology 3:1-61). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al. (1983) J. Virol., 45:555-564; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al. (2004), J. Virol., 78:6381-6388; the AAV-10 genome is provided in Mol. Ther., 13 (1): 67-76 (2006); and the AAV-11 genome is provided in Virology (2004), 330 (2): 375-383. The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, (1992) Current Topics in Microbiology and Immunology, 158:97-129.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., (1996) Hum Gene Ther, 8:659-669 (1997); Kessler et al., Proc Nat. Acad Sc. USA, 93:14082-14087; and Xiao et al., (1996) J Virol, 70:8098-8108. See also, Chao et al., Mol Ther, (2000), 2:619-623 and Chao et al., (2001) Mol Ther, 4:217-222. Moreover, because muscle is highly vascularized, recombinant AAV transduction has resulted in the appearance of transgene products in the systemic circulation following intramuscular injection as described in Herzog et al., (1997) Proc Natl Acad Sci USA, 94:5804-5809 and Murphy et al., (1997) Proc Natl Acad Sci USA, 94:13921-13926. Moreover, Lewis et al., (2002) J Virol, 76:8769-8775 demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the disclosure comprise, or consist essentially of, or yet further consist of a nucleic acid molecule encoding γ-sarcoglycan and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of rAAV variants, for example rAAV with capsid mutations, are also contemplated. See, for example, Marsic et al., (2014), Molecular Therapy, 22(11): 1900-1909. The nucleotide sequences of the genomes of various AAV serotypes are known in the art. To promote skeletal muscle specific expression, AAV1, AAV5, AAV6, AAV8 or AAV9 may be used.

In some embodiments, a regulatory element is operably linked to one or more elements of a gene-editing system so as to drive expression of the one or more elements of the gene-editing system.

The gene-editing systems described herein can further comprise one or more labels or detection tags (e.g., FLAG™ tag, epitope or protein tags, such as myc tag, 6 His, and fluorescent fusion protein). In an aspect, the label (e.g., FLAG™ tag) is fused to the NLS. In an aspect, the disclosed methods and compositions further comprise a fusion protein, or a polynucleotide encoding the same. In various aspects, the fusion protein comprises at least one epitope-providing amino acid sequence (e.g., “epitope-tag”), wherein the epitope-tag is selected from i) an epitope-tag added to the N- and/or C-terminus of a protein, or ii) an epitope-tag inserted into a region of a protein, and an epitope-tag replacing a number of amino acids in a protein.

As used herein, “epitope tags” refer to short stretches of amino acids to which a specific antibody can be raised, which in some respects allows one to specifically identify and track the tagged protein that has been added to a living organism or to cultured cells. Detection of the tagged molecule can be achieved using a number of different techniques. Examples of such techniques include immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975), Remington's Pharm. Sci., 15th Ed. (Mack Publ. Co., Easton).

As used herein, the term “detectably labeled” means that the agent (biologic or small molecule) is attached to another molecule, compound or polymer that facilitates detection of the presence of the agent in vitro or in vivo. A “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996), Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. 1996), Handbook of Fluorescent Probes and Research Chemicals (6^{th} ed.).

### MODES FOR CARRYING OUT THE DISCLOSURE

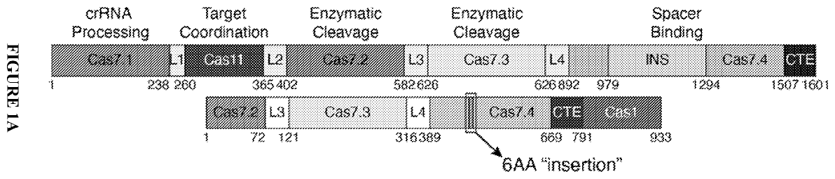

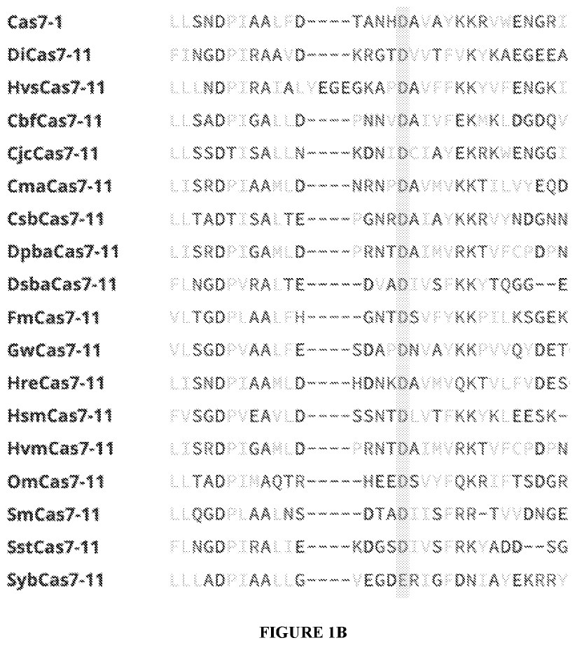

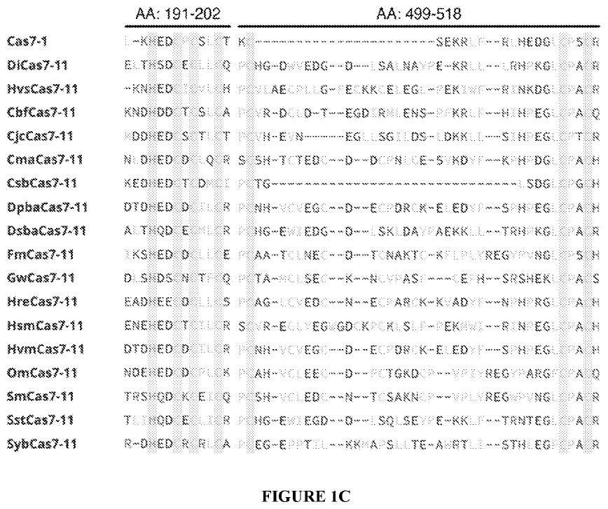

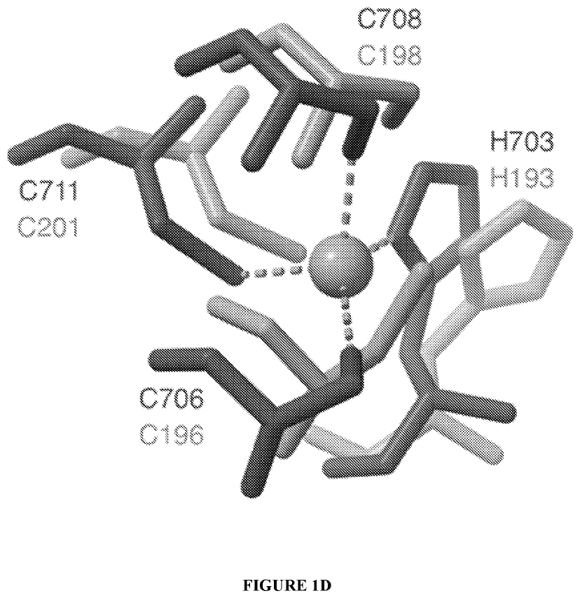

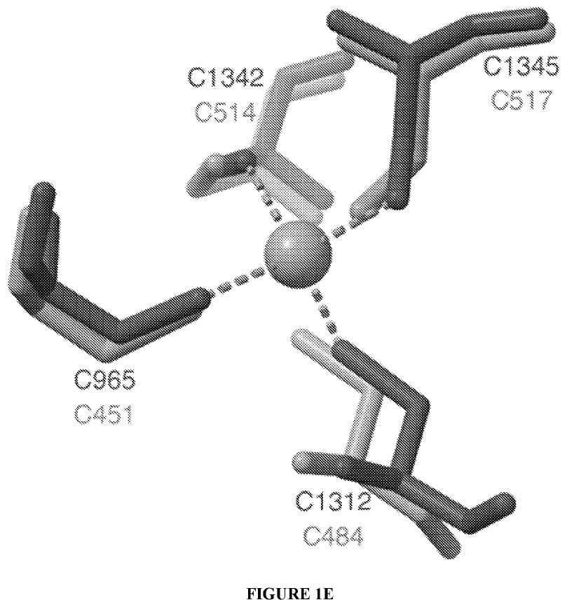

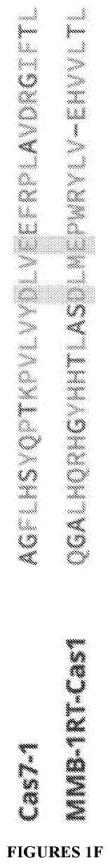

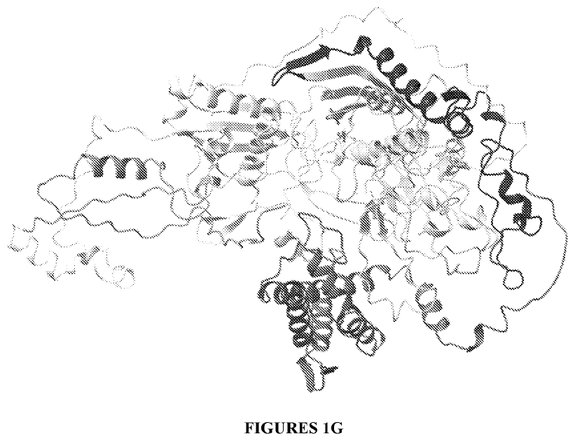

As new discoveries of CRISPR-Cas systems emerge, the continuous evolution of these enzymes reveals new possibilities for generating new genome engineering technologies. Type III-E CRISPRCas Effectors have commonly been characterized as Cas7-11 effectors. An example of a Cas7-11 effector is shown in FIG. 1A. These effectors operate as single-effector Cas proteins, and are comprised of multiple domains recombined into one protein: 4 Cas7 domains, an insertion domain (INS), a single Cas11 domain, a C-terminal domain, and multiple linkers. The size of known Cas7-11 effectors typically ranges from 1300-1800 amino acids. Described herein is a previously uncharacterized polypeptide composition, termed Cas7-1 (SEQ ID NO: 1), where the Cas1 domain appears to operate similarly to the Cas11 domain. Cas7-1 is also the first example of a type III-like effector naturally lacking an INS domain and demonstrating the possibility of a naturally compact type III-E effector. A type III-E effector is a type of type III effector. Using both Cas7-1 and the Cas7-11 architecture, in one aspect, Applicant developed orthologous domain substitution (“ODS”) to generate synthetic type III-E effectors capable of RNA-targeting. The ODS method reveals a unique modularity of the type III-E effector class, where orthologous domains are often interchangeable and minimally impact RNA knockdown activity. Furthermore, Applicant demonstrates the applicability of this design method for generating the most compact type III-E effector currently known.

Along with presenting an engineering method that manipulates the modularity of Cas7-11 proteins, Applicant also uncovered a unique crRNA processing activity where the mature crRNA contains a spacer surrounded by fragments of the DR on both the 5′ and 3′ end. This mode of crRNA processing has not been reported for type III-E or other single-effector CRISPR-Cas systems capable of array processing (22, 23), and is reminiscent of crRNA processing by multiprotein type I CRISPR-Cas systems (20). For synthetic Cas7 (herein referred to as Cas7-S or SynCas) effectors, Applicant found this crRNA processing activity to directly impact both array processing and targeting activity and Applicant provides a solution for crRNA array design when applying Cas7-S effectors for RNA-targeting applications using a mature direct repeat (“mDR”) array structure.

In several aspects, the ODS method and Cas7-S effectors are useful for protein and transcriptome engineering, respectively.

The following reference is incorporated herein by reference in its entirety: Brogan, D J, et. al “Synthetic type III-E CRISPR-Cas effectors for programmable RNA-targeting” (2024) bioRxiv https://doi.org/10.1101/2024.02.23.581838.

Applicant describes herein synthetic proteins and a CRISPR system including the synthetic polypeptides. Applicant also describes herein methods of targeting RNA with the CRISPR system to treat microsatellite repeat expansion (MRE) disorders and viral diseases of plants and animals.

The unique architecture of Cas7-11 characterized herein suggests a unique modularity of type III-E effectors that can be exploited for engineering.

### Synthetic Protein and Vector Comprising Synthetic Protein

Applicant provides herein a synthetic or engineered protein comprising, or consisting essentially of, or yet further consisting of a Cas7 peptide and either a Cas1 peptide or Cas11 peptide, wherein the synthetic effector has RNA-targeting function. As referenced herein, the synthetic or engineered protein is also referred to as SynCas or Cas7-S, or as a synthetic effector protein. As used herein, the peptides that make up the synthetic protein as well as CRISPR-Cas Type III-E CRISPR-Cas effectors are also referred to as domains. That is, in the context of the synthetic proteins and Type III-E CRISPR-Cas effectors the terms “peptide” and “domain” are used interchangeably. For example, the Cas7.1 peptide is also referred to as the Cas7.1 domain.

More specifically, Applicant provides a synthetic protein comprising a first Cas7 peptide and either a Cas1 peptide or a Cas11 peptide, wherein the first Cas7 peptide is Cas7.1. In one embodiment, the Cas1 peptide or Cas11 peptide is C-terminal relative to the first Cas7 peptide (i.e. the domains are arranged so the first Cas7 peptide is towards the N-terminus and the Cas1 or Cas11 peptide relative to the first Cas7 peptide is towards the C-terminus of the protein). In another aspect, the synthetic protein includes at least two Cas7 peptides. In another aspect, the synthetic protein includes a second, third, and fourth Cas7 peptide, optionally wherein the second Cas7 peptide is Cas7.2, the third Cas7 peptide is Cas7.3, and the fourth Cas7 peptide is Cas7.4. In one embodiment, the second, third, and fourth Cas7 peptides are C-terminal relative to the first Cas7 peptide and the Cas1 or Cas11 peptide.

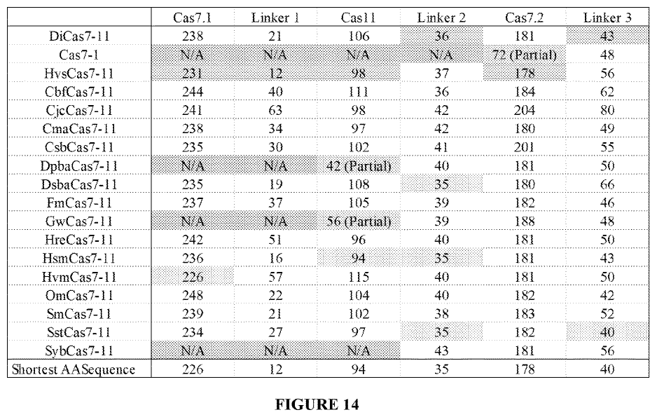

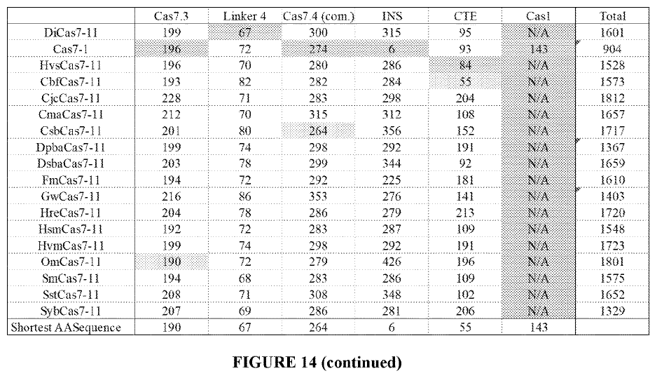

The Cas7 peptides can have lengths according to the sizes of peptides shown in FIG. 14. However, Cas7 peptide size can vary depending on the Type III-C CRISPR-Cas Effectors selected to build the synthetic proteins.

In one aspect the synthetic protein includes an INS domain inserted in the fourth Cas7 peptide Cas7.4. The INS domain is found in all known CRISPR-Cas Type III-E effectors. The INS derived from the Cas7-1 effector is only 6 amino acids in length while the INS from the Cas7-11 effectors is longer. The length of the INS domain in the synthetic protein can vary depending on the Type III-C CRISPR-Cas Effectors selected to build the synthetic proteins. In some embodiments the synthetic protein includes a CTE domain. The CTE domain size can vary depending on the Type III-C CRISPR-Cas Effectors selected to build the synthetic proteins.

The domains of the synthetic proteins have different roles. For example, Cas7.1 is involved in crRNA processing, Cas11 and Cas1 are involved in target coordination, Cas7.2 is involved in enzymatic cleavage, and Cas7.3 is involved in enzymatic cleavage (see FIG. 1A).

The synthetic protein is designed to be compact and modular, wherein the synthetic protein includes peptides from multiple Cas7-11 and/or Cas7-1 effector proteins (collectively referred to as Type III-E CRISPR-Cas effectors). All of the synthetic proteins provided herein are derived from Cas7-11 and/or Cas7-1 effector proteins. The Cas7-1 effector protein has a Cas1 domain and the Cas7-11 effector has a Cas11 domain. In one aspect, the Cas7-11 effector is selected from DiCas7-11 and HvsCas7-11. In other aspects, the Cas7-11 effector can be selected from other Cas7-11 effectors including, but not limited to, CbfCas7-11, CjcCas7-11, CmaCas7-11, CsbCas7-11, DpbaCas7-11, DsbaCas7-11, FmCas7-11, GwCas7-11, HreCas7-11, HsmCas7-11, HvmCas7-11, OmCas7-11, SmCas7-11, SstCas7-11, and SybCas7-11. Exemplary sequences for these effectors can be found in Table 1 (SEQ ID NOs: 1-18). The domain information for the Type III-E CRISPR-Cas effectors is found in FIG. 14. Cas7.4 (com) indicates that the domain is not broken up into two sections (as is seen in FIG. 17 for the synthetic protein domains). As seen in FIG. 14 and Table 1, the length of the Type III-E CRISPR-Cas effector matches the sum of the individual domains. Therefore, sequence of an individual domain, except for the exact placement of the INS domain within the Cas7.4 domain, can be mapped onto the sequence. For example, in DiCas7-11:

Cas7.1 is Amino Acids (AA) 1-238; Linker 1 (L1) is AA 239-259; and Cas11 is AA 260-365. Type III-E CRISPR-Cas effectors are known in the art. NCBI Reference Sequences that match to 100% identity in a BLASTP search are given in Table 1.

In one aspect, the effectors described in Table 1 have domain sizes (amino acid) according to FIG. 14. In one aspect, the Cas7-11 effectors can have sequences that comprise, or consist essentially of, or yet further consist of the effectors found in Table 1.

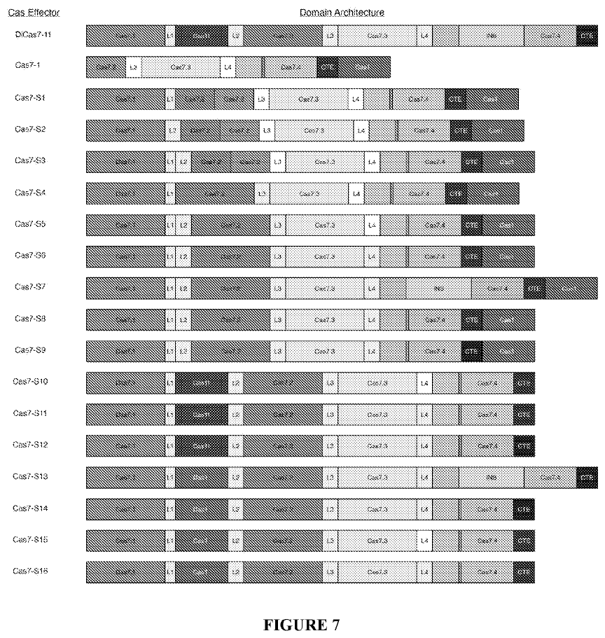

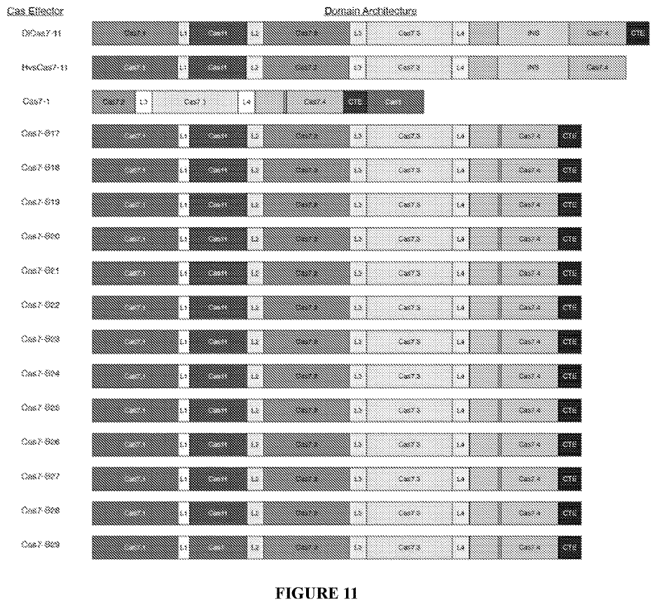

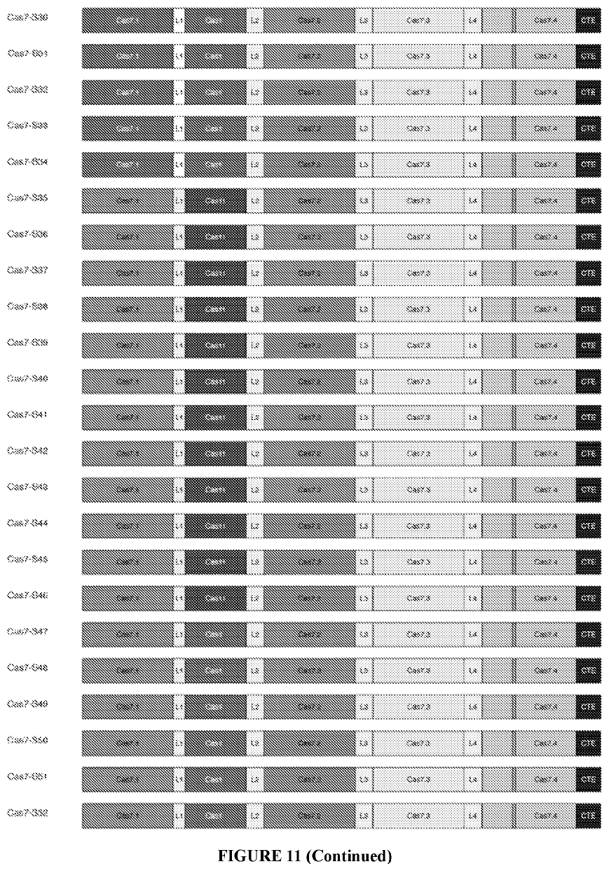

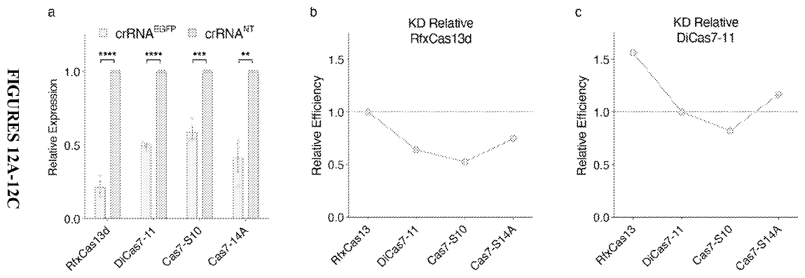

According to one embodiment, all of the domains (Cas7 domains, linker domains, INS domain, and CTE domain) in the synthetic proteins are derived from Cas7-11 and/or Cas7-1 effector proteins. In one aspect, the synthetic proteins are derived from two Cas7-11 and/or Cas 7-1 effector proteins. In another aspect the synthetic proteins are derived from three Cas7-11 and/or Cas7-1 effector proteins. In still other aspects the synthetic proteins are derived from more than three Cas7-11 and/or Cas7-1 effector proteins. For example, the synthetic protein Cas7-S54 (SynCas.v54, SEQ ID NO: 47) has a domain breakdown of: Cas7.1 (DiCas7-11), Linker 1 (DiCas7-11), Cas11 (HsmCas7-11), Linker 2 (HsmCas7-11), Cas7.2 (HvsCas7-11), Linker 4 (SstCas7-11), Cas7.3 (OmCas7-11), Linker 4 (DiCas7-11), Cas7.4 (1&2) (CsbCas7-11), 6 Amino Acid INS (Cas7-1), CTE (CbfCas7). The domain information for the synthetic proteins as described herein are found in FIG. 7, FIG. 11, FIG. 17, Table 2 (SEQ ID NO: 19-47), and Table 6. As seen in the embodiments in FIGS. 7 and 11, the configuration of the synthetic protein can vary. For example, the Cas1 peptide can be at the C-terminus or positioned between linkers at the N-terminus end of the protein.

Cas7-11 has a domain layout according to the schematic of FIG. 1A. Cas7-1 has a domain layout according to the schematic of FIG. 1A. As seen in FIG. 1A, Cas7-1 has a partial Cas7.2 domain similar to Cas7-11, a small (6 amino acid) INS segment in the Cas7.4 domain rather than the INS domain in Cas7-11, and a Cas1 nuclease domain at the C-terminus. As compared to Cas7-11, Cas 7-1 does not include a Cas7.1 domain, which is the domain responsible for crRNA processing, a portion of the Cas7.2 domain which contains a catalytic residue, two linker domains. Cas7-11 includes a Cas11 domain, while Cas7-1 includes a Cas1 domain. A comparison of the Cas7-1 and Cas7-11 domains in multiple effectors is shown in FIG. 14. The Cas7-1 and Cas7-11 domains in different effectors can vary in size, as well as presence or absence. In one aspect, the Cas7-11 effector used to make the synthetic proteins is selected from DiCas7-11 and/or HvsCas7-11. In other aspects, the Cas7-11 effector can be selected from DiCas7-11 and/or HvsCas7-11 as previously mentioned and/or additional Cas7-11 effectors including, but not limited to, CbfCas7-11, CjcCas7-11, CmaCas7-11, CsbCas7-11, DpbaCas7-11, DsbaCas7-11, FmCas7-11, GwCas7-11, HreCas7-11, HsmCas7-11, HvmCas7-11, OmCas7-11, SmCas7-11, SstCas7-11, and/or SybCas7-11. Exemplary sequences for these Cas7-11 effectors can be found in Table 1 (SEQ ID NOs: 2-18). In some aspects, the Cas7-11 effectors used to create the synthetic proteins comprise, or consist essentially of, or yet further consist of any one or more of the sequences in Table 1 (SEQ ID NOs: 2-18). In some aspects, the Cas7-1 effector comprises, or consists essentially, of, or yet further consists of SEQ ID NO: 1 (Table 1).

As seen in FIG. 7 and FIG. 11, in one aspect the synthetic protein includes linkers (also referred to herein as linker domains), optionally one, optionally, two, optionally, three, or optionally 4 linkers. The configurations of the linker domains can vary according to the synthetic protein. The linkers can vary in length, but in some aspects may be the length according to the linkers described in FIG. 14. In some aspects the linkers are between 10 and 70 amino acids in length. However, linker size can vary depending on the Type III-C CRISPR-Cas Effectors selected to build the synthetic proteins. In one embodiment, the synthetic protein includes a first linker located between the first Cas7 peptide and either the Cas1 peptide or Cas11 peptide, further including a second linker located between either the Cas1 peptide or Cas11 peptide and the second Cas7 peptide, a third linker located between the second Cas7 peptide and the third Cas7 peptide, and a fourth linker between the third Cas7 peptide and the fourth Cas7 peptide, optionally wherein the first, second, third, and fourth linkers are the same or different. In another aspect, the synthetic protein includes a first linker located between the first Cas7 peptide and the second Cas7 peptide, further including three additional linkers. In some embodiments, the synthetic protein includes three linkers. In some embodiments the first and second linker are adjacent, optionally wherein the first and second linker are located between the first Cas7 peptide and the second Cas7 peptide. The linkers are derived from at least one Cas7-11 and/or Cas7-1 effector. In some embodiments the linkers are derived only one Cas7-11 or Cas7-1 effector. Exemplary linker position in the synthetic proteins can be found in FIG. 7, FIG. 11, and FIG. 17.

In some embodiments, the synthetic protein is between about 1100 and about 1500 amino acids, between about 1200 and about 1400 amino acids, between about 1200 and about 1300 amino acids, or about 1300 amino acids in length.

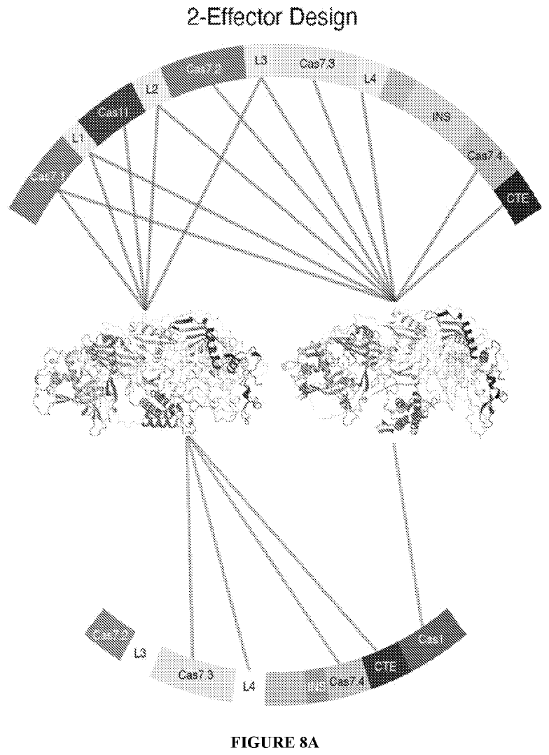

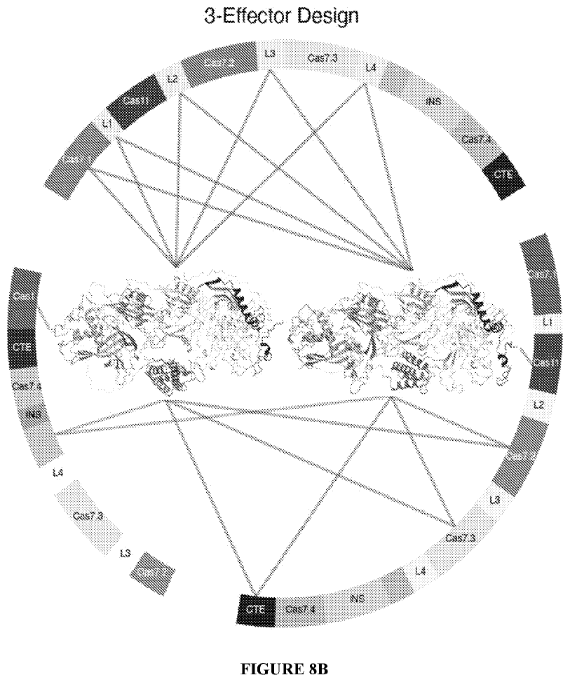

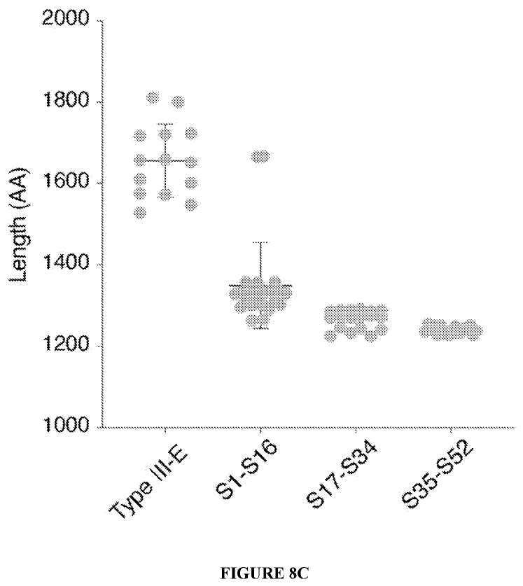

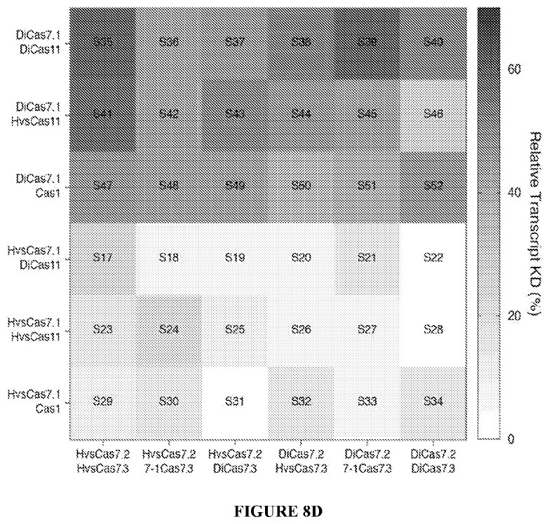

The synthetic proteins can be configured as shown in FIG. 7, FIG. 8, and FIG. 11. As seen in FIG. 7 and FIG. 11 and Tables 2 the domains of the synthetic proteins can be derived from multiple effectors, and the domains can vary in size. Further, the synthetic proteins sequences are in Table 2 (SEQ ID NOs: 19-54). The origin of each domain (i.e. which Type III-E CRISPR-Cas effector the domain is derived from) is indicated in Table 2. The individual domains (amino acid boundaries) are indicated in FIG. 17. In some embodiments, the synthetic protein comprises an amino acid sequence comprising at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of any one of SEQ ID NO: 19, 20, 21, 22, 23, 24, 25, 26, 27, 28, 29, 30, 31, 32, 33, 34, 35, 36, 37, 38, 39, 40, 41, 42, 43, 44, 45, 46, or 47. In some embodiments the synthetic protein comprise, or consist essentially of, or yet further consists of any one of SEQ ID NO: 19, 20, 21, 22, 23, 24, 25, 26, 27, 28, 29, 30, 31, 32, 33, 34, 35, 36, 37, 38, 39, 40, 41, 42, 43, 44, 45, 46, or 47.

While most of the synthetic proteins included in Table 2 (domain information shown in FIG. 17) included domains from only two or three Type III-E CRISPR-Cas effectors (DiCas7-11, HvsCas7-11, and Cas7-1), other Type III-E CRISPR-Cas effectors can be used as shown in Cas7-S54 (SEQ ID NO: 47). The included synthetic proteins are intended to be exemplary, not limiting. In some embodiments the synthetic proteins can have domain configurations that are not shown in FIGS. 7 and 11 and Table 2, but include four Cas7 peptides (Cas7.1, Cas7.2, Cas7.3, and Cas7.4), either a Cas1 or Cas11 peptide, an INS peptide, and a CTE peptide.

Synthetic proteins not included in Table 2 also have domain information included in Table 6. While the sequences are not included, Applicant shows the variability of domains that can exist in the synthetic proteins. In one aspect, the synthetic proteins are compact enough to package into a vector. According to one embodiment, a vector is comprised of the synthetic proteins (SynCas) described herein. Optionally, the vector is, comprises or is derived from a plasmid, an adenovirus, an adeno-associated virus (AAV), a retrovirus, a herpes simplex virus, a human immunodeficiency virus (HIV), or a synthetic vector.

In one aspect, the vector is a plasmid wherein the plasmid includes a CMV promoter and bGH terminator. In one aspect the vector is an AAV vector. The plasmids described herein in Table 4 are available at addgene.org.

In a further aspect, the synthetic protein or vector is detectably labeled. The detectable label can be an radioisotopes, fluorochromes, chemiluminescent compounds, dyes, and proteins, including enzymes.

### CRISPR System

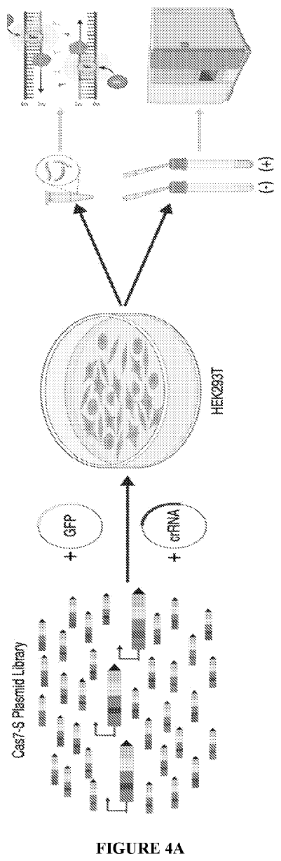

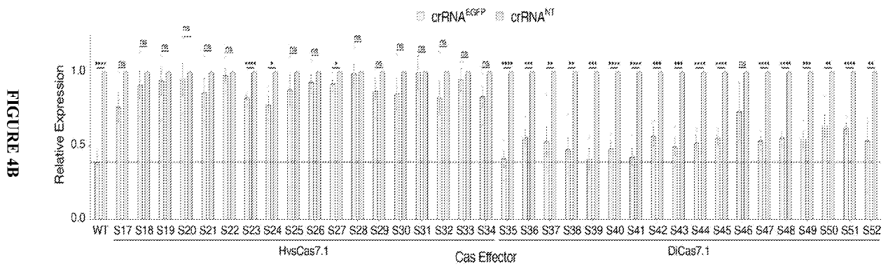

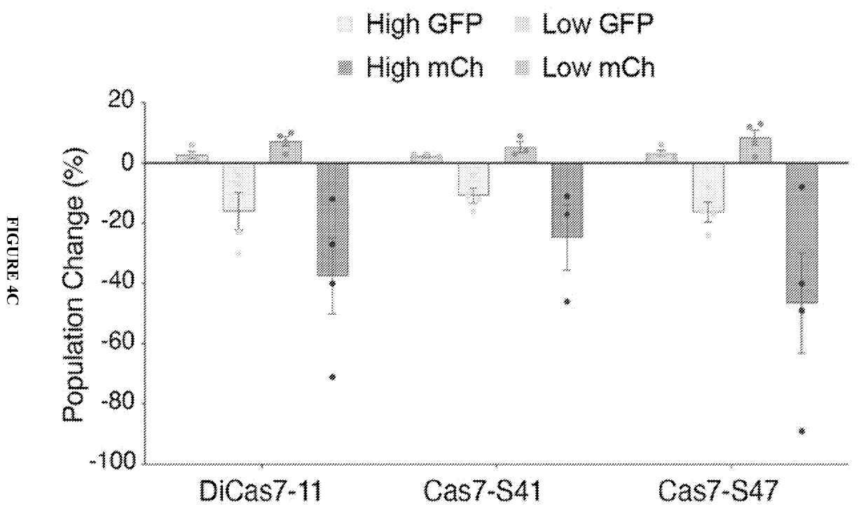

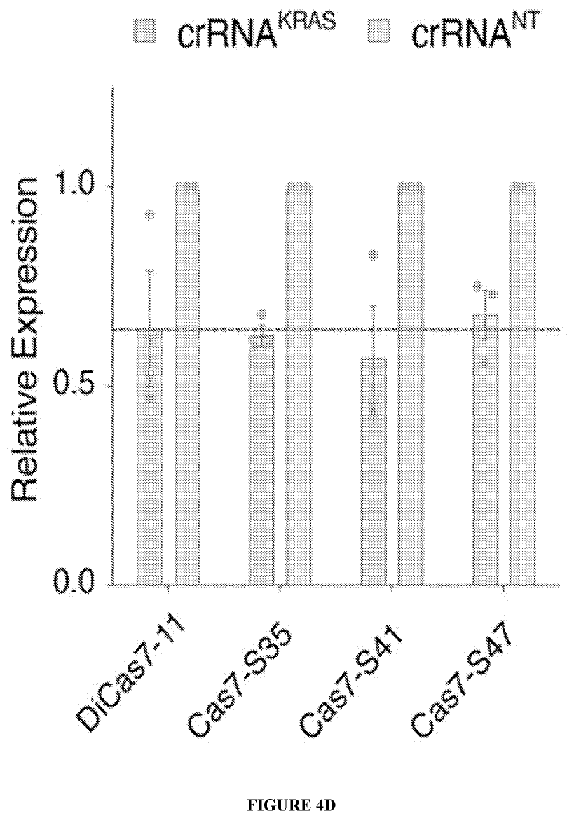

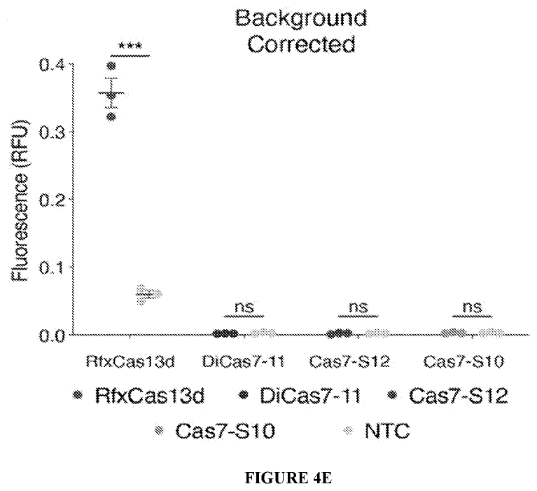

Also provided herein is a CRISPR system (referred to interchangeably as a CRISPR-Cas system) which in some embodiments includes the synthetic proteins and/or vectors including the synthetic proteins described herein and CRISPR RNA (crRNA). As shown in FIG. 4B, the crRNA is combined with the synthetic protein (Cas7-S, also referred to as SynCas) to form the CRISPR system. Both the crRNA and synthetic proteins can be packaged in a plasmid or vector. In some embodiments the crRNA and synthetic protein are packaged in the same vector and in other embodiments the crRNA and synthetic protein are packaged in different vectors and delivered simultaneously or sequentially. For example, the crRNA in the CRISPR system can be delivered in a plasmid, wherein the plasmid includes a U6 promoter and terminator and the synthetic protein is delivered in an AAV vector. In some embodiments, the CRISPR system includes more than one synthetic protein. In some embodiments, the CRISPR system includes more than one crRNA.

In some embodiments, the crRNA can recognize a target RNA in an organism (for example, a human) or cell, for example, by hybridizing to the target RNA. In some embodiments, the crRNA comprises a sequence that is complementary to the target RNA. In alternative embodiments, a variety of RNA targets can be recognized by the crRNA. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, viral noncoding RNA, or the like. In some embodiments, a target RNA can be an RNA involved in pathogenesis or a therapeutic target for conditions such as cancers, neurodegeneration, cutaneous conditions, endocrine conditions, intestinal diseases, infectious conditions, neurological disorders, liver diseases, heart disorders, autoimmune diseases, or the like. In some embodiments, the target RNA is in a patient in need of treatment, such as a human suffering from a disease or disorder.

Exemplary target sequences are included in Table 3. In some embodiments, the RNA sequence is from a pathogen, more specifically a ssRNA viral pathogen, for example severe acute respiratory syndrome coronavirus 2 (SARS-COV-2) or deformed wing virus (DVW). The genome of SARS-COV-2 is known in the art, for example in NCBI Reference Sequence NC_045512.2 or as disclosed in PCT/US2021/030953. The genome of DVW is known in the art, for example NCBI Genbank Accession No. KY909333.1. In other embodiments the target sequence is derived from a mammal or other animal. In some embodiments the target sequence is from a gene associated with a microsatellite repeat expansion (MRE) disorder. MRE disorders include but are not limited to Huntington's Disease, Amyotrophic lateral sclerosis, Fragile X syndrome, spinal muscular atrophy, and myotonic dystrophy. The genes associated with these MRE disorders are known in the art. The genes below are intended to be representative, not exhaustive. For example:
Huntington's Disease: HTT gene (Nopoulos, (2016) Dialogues Clin. Neurosci. 18 (1): 91-98, 10.31887/DCNS.2016.18.1/pnopoulos);
Amyotropic Lateral Sclerosis: SOD1, VCP, OPTN, UBQLN2, PFN1, DCTN1, ELP3, C9ORF72 (Ghasemi and Brown, (2018) Cold Spring Harb. Perspect. Med. 8(5): a024125, 10.1101/cshperspect.a024125);
Fragile X Syndrome: FMRI (Protic et al., (2022) Int. J. Mol. Sci. 23 (4): 1935, 10.3390/ijms23041935);
Spinal muscular atrophy: SMN1 (D'Amico et al., (2011) Orphanet Journal of Rare Diseases 6 (71), https://doi.org/10.1186/1750-1172-6-71); and
Myotonic dystrophy: DPMK (Soltanzadeh, (2022) Genes (Basel) 13 (2): 367, 10.3390/genes13020367).

The target sequence can also be from a plant. In some embodiments, the sequence in a plant is associated with disease susceptibility. The sequence can also be from a plant pathogen. CRISPR/Cas9 and CRISPR/Cpf1 genome editing in plants and in plant pathogens is known in the art, and could be translated to the CRISPR system describe herein (see e.g. Paul et al., (2021) Front. Plant Sci. 12: article 700925.

In one aspect, the crRNA is comprised of a direct repeat and a spacer sequence, wherein the spacer sequence is about 20 to about 30 nucleotides in length, optionally wherein the spacer is about 22 nucleotides to about 24 nucleotides in length. The spacer sequence can be complementary to a target RNA sequence in a target organism or cell. In some aspects, the direct repeat comprises an amino acid sequence comprising at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of any one of SEQ ID NO: 75, 76, or 77 (Table 5). In some aspects, the crRNA includes the spacer sequence surrounded by fragments of the direct repeat at both the 5′ end and the 5′ end. According to some embodiments, the crRNA comprises, or consists essentially of, or yet further consists of the sequence of any one of SEQ ID NOs: 48, 49, 50, 51, 52, 53, 54, 55, 56, 57, 58, 59, 60, 61, 62, 63, 64, 65, 66, 67, 68, 69, 70, 71, 72, 73, or 74 (Table 3).

In some embodiments, the crRNA or the direct repeat are represented herein as a DNA sequence encoding the crRNA or the direct repeat. As it would be understood by one of skill in the art, a crRNA as disclosed herein can be substituted by a polynucleotide encoding such crRNA, thereby the encoded crRNA can be used in a system or a method as disclosed herein. According to some embodiments, a polynucleotide encoding the crRNA is added along with other reagents necessary for transcribing the polynucleotide to the crRNA, such as RNA polymerase, ATP, GTP, UTP, CTP, a primer pair consisting of a reverse primer and a forward primer, and a buffer suitable for the transcription, thus producing the crRNA.

According to some embodiments, the CRISPR system can further include a detectable label. The detectable label can be an radioisotopes, fluorochromes, chemiluminescent compounds, dyes, and proteins, including enzymes. In some embodiments the detectable label, crRNA, and synthetic protein are packaged in the same vector. In other embodiments the detectable label, crRNA, and synthetic protein are packaged in two or more vectors and are delivered simultaneously or sequentially.

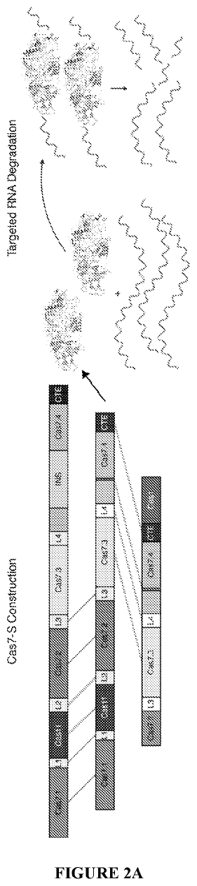

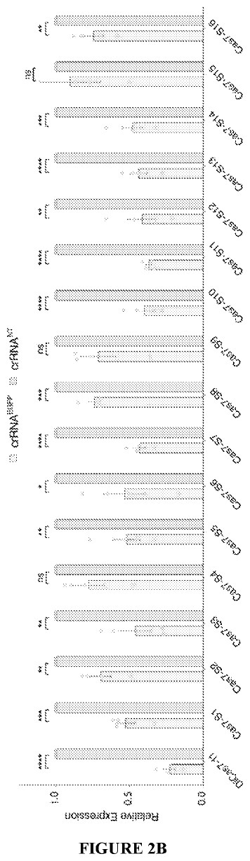

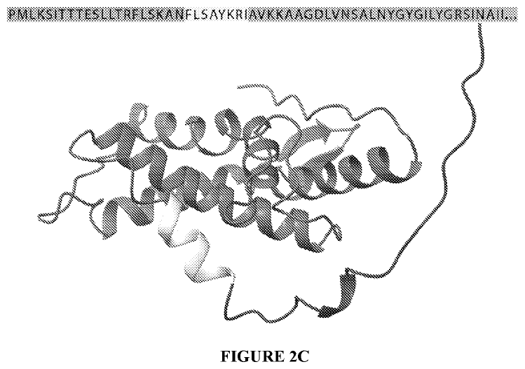

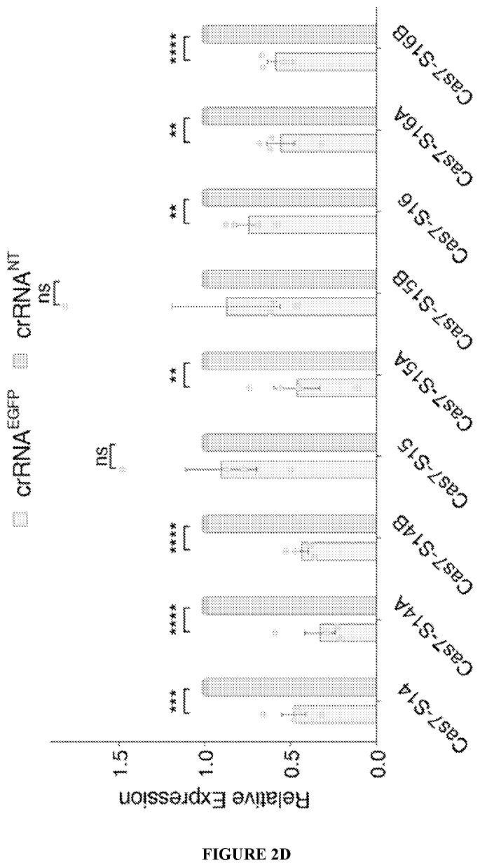

As shown in FIG. 8D, which shows a heatmap of RNA knockdown (of RNA target EGFP) for multiple Cas7-S synthetic effector proteins tested in a CRISPR system, and FIG. 4B, which shows knockdown of the synthetic proteins tested in a CRISPR system as compared to wild type DiCas7-11, the RNA knockdown activity varies depending on the synthetic protein. The synthetic proteins in these images include domains from three effector proteins: DiCas7-11, HvsCas7-11, and Cas7-1. Similar variable activity is seen in FIG. 2B, which shows RNA knockdown (of RNA target EGFP) for multiple Cas7-S synthetic proteins tested in a CRISPR system as compared to wild type DiCas7-11 for synthetic proteins which include domains from two effector proteins: DiCas7-11 and Cas7-1.

For the synthetic proteins including domains from three effectors, Cas7-S35, Cas7-S36, Cas7-S37, Cas7-S38, Cas7-S39, Cas7-S40, Cas7-S41, Cas7-S42, Cas7-S43, Cas7-S44, Cas7-S45, Cas7-S47, Cas7-S48, Cas7-S49, Cas7-S51, and Cas7-S52 had the best knockdown activity. Cas7-S35, Cas7-S39, and Cas7-S41 had activity comparable to wild type DiCas7-11 (see FIG. 8D). Sequences for these synthetic proteins are in Table 2 and domain information is in FIG. 17.

For the synthetic proteins including domains from two effectors, Cas7-S3, Cas7-S7, Cas7-S10, Cas7-S11, Cas7-S12, Cas7-S13, and Cas7-S14 had the best knockdown activity (see FIG. 2B). Sequences for these synthetic proteins are in Table 2 and domain information is in FIG. 17.

Further, truncation of the Cas7-1 protein can affect the knockdown activity of a synthetic effector, as seen in FIG. 2D (Cas7-S15, Cas7-S15A, Cas7-S15B). Sequences for these synthetic proteins are in Table 2 and domain information is in FIG. 17.

In one aspect, the synthetic protein includes both a Cas7.2 domain derived from HvsCas7-11 and a Cas7.3 domain derived from HvsCas7-11. In one aspect the synthetic protein includes Cas7.1 domain derived from DiCas7-11. In one aspect the synthetic protein includes a Cas11 domain from DiCas7-11 or HvsCas7-11. In one aspect the synthetic protein has a Cas1 domain is located between L1 and L2. In another aspect the synthetic protein includes a DiCas7-11 derived INS domain and a Cas1 domain. Proteins according to the aforementioned aspects had increased knockdown activity compared to other synthetic effectors (FIG. 2B, FIG. 4B, FIG. 7, FIG. 8D, and Table 2).

### Compositions Including the CRISPR System

Also provided herein are compositions including the synthetic proteins and/or vectors and/or CRISPR systems as described herein. In some embodiments, the composition includes a carrier. In some aspects the carrier is a pharmaceutically acceptable carrier.

The compositions described herein can be manufactured in conventional manners including by means of mixing, dissolving, granulating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, subcutaneous, oral, buccal, rectal, sublingual, or transdermal administration routes. In some cases, parenteral administration comprises, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, 40ocusate40pyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides, or the synthetic proteins as described herein.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium 44ocusate, triacetin, Tween 80, vitamin E TPGS, ammonium salts and the like.

The pharmaceutical compositions for the administration can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology can take a form suitable for virtually any mode of administration, including, for example, topical, ocular, oral, buccal, systemic, nasal, injection, infusion, transdermal, rectal, and vaginal, or a form suitable for administration by inhalation or insufflation.

### Methods of Use

Also provided herein are methods to target and cleave an RNA molecule with the CRISPR system and/or compositions described herein. In some embodiments, the CRISPR system is delivered by contacting the RNA with the CRISPR system or composition in vitro. The RNA can be in a cellular genome. In another aspect, the CRISPR system is delivered by contacting a cell with the CRISPR system or composition in vivo. Contacting the cell with the CRISPR system and/or composition can cleave a target RNA molecule in the cell, optionally wherein the target RNA molecule encodes a reporter gene or SARS-Cov-2 gene. The CRISPR system and/or composition can knockdown the RNA and inhibiting or reducing expression of the RNA. In some embodiments, the target RNA is fragmented, and in other embodiments the splicing of the target RNA is modified. The cells can be from an animal, such as a human. In another aspect, the cells can be from a plant. The cells can be commercially available form the American Type Culture Collection (ATCC). The cell can be a prokaryotic cell such as an E. coli cell or a eukaryotic cell such as a mammalian or human cell, or plant cell. In one aspect, the cell is HEK293T.

In some embodiments, the CRISPR system and/or compositions can be used to target and cleave an RNA molecule in a virus, for example a ssRNA. ssRNA viruses include but are not limited to SARS-COV-2, DVW. Contacting the virus with the CRISPR system and/or composition can cleave a target RNA molecule in the ssRNA virus, thereby knocking down the RNA and inhibiting or reducing RNA expression.

Further, this application provides methods to treat a microsatellite repeat expansion (MRE) disorder in a subject in need thereof, which includes administering to a subject in need thereof an effective amount of the CRISPR system and/or composition. Target RNA in the subject in need thereof can be modified (knocked down) with the CRISPR system and/or composition. More specifically, the CRISPR system and/or composition can be delivered to the central nervous system of the subject, for example through intrathecal administration. The CRISPR system can include a crRNA complementary to a target sequence or target sequences in a subject. The target sequence or target sequences can be genes or parts of genes associated with a MRE disorder. Genes associated with MRE disorders are known in the art. The MRE disorder can be selected from Huntington's Disease, Amyotrophic lateral sclerosis, Fragile X syndrome, spinal muscular atrophy, and myotonic dystrophy. The CRISPR system can also be used to treat additional diseases such as cancer. The use of CRISPR systems are known in the art to target and treat MRE disorders in subjects in need, and include Morelli et al. (2023) nature neuroscience 26, 27-38, https://doi.org/10.1038/s41593-022-01207-1; and Powell et al. (2022) Science Advances 8 (3), https://doi.org/10.1126/sciadv.abk2485, the contents of which are hereby incorporated by reference in their entirety. The methods described can be adapted to the CRISPR system as described herein

The methods are useful to treat subjects in need such as mammals for example humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a MRE disorder. The methods are also useful to treat non-mammal subjects, for example bees infected with the Deformed Wing Virus.

In further aspects, the CRISPR system and/or composition can be used to target an RNA virus infected in a subject in need thereof. For example, the CRISPR system can be used to target a virus such as DVW in a bee. For example, the CRISPR system and/or composition can be added to queen bee or other bee or insect food. The CRISPR system will be taken up by the bee upon ingestion of the food. In one embodiment, the CRISPR system can be designed to target a sequence in the bee genome associated with DWV susceptibility. In another embodiment, the CRISPR system can be designed to target a sequence in the DWV genome associated with pathogenicity.

For the above methods, an effective amount is administered, and administration of the cell or population serves to attenuate any symptom or prevent additional symptoms from arising. When administration is for the purposes of preventing or reducing the likelihood of cancer, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal. Preferably the composition is administered intrathecally.

Also provided herein are methods to treat a disease in a plant, which comprises administering to the plant an effective amount of the CRISPR system and/or the composition as described herein. Administration of the CRISPR system and/or the composition can be done in situ. For example, the CRISPR system and/or the composition can be added to water and applied as a drench to the plant or soil the plant is in. The disease can be a viral disease, for example a disease caused by a plant virus. The CRISPR system can target a sequence in the plant virus associated with pathogenicity, for example a ssRNA virus including but not limited to Soybean Mosaic Virus, Potato Virus Y, Rice Stripe Virus, and Maize Mosaic Virus. The target RNA sequence in the virus is knocked down thereby protecting the plant. In alternative embodiments, the methods described herein can also be used to treat a bacterial or fungal disease The CRISPR system can target a sequence in the bacterial or viral plant pathogen associated with pathogenicity. In an alternative embodiment, an RNA sequence or sequences associated with plant pathogen susceptibility in the plant genome can also be targeted, thereby knocking down RNA expression and increase plant disease resistance against bacterial, fungal, or viral diseases. The plant can include, but is not limited to commercial crops such as rice, maize, soy, or potatoes. The use of CRISPR systems for in planta virus interference is known in the art, for example in Mahas et al. (2019) Genome Biology 20, https://doi.org/10.1186/s13059-019-1881-2 and Aman et al. (2018) Genome Biology 19, https://doi.org/10.1186/s13059-017-1381-1. Further, CRISPR/Cas9 and CRISPR/Cpf1 genome editing in plants and in plant pathogens is known in the art. (see e.g. Paul et al., (2021) Front. Plant Sci. 12: article 700925. These references to CRISPR editing in plants are hereby incorporated by reference in their entirety. The methods described could be adapted to the CRISPR systems described herein.

The following examples are intended to illustrate, and not limit the embodiments disclosed herein.

### Example 1—Synthetic Type III-E CRISPR-Cas Effectors for Programmable RNA-Targeting

Without wishing to be bound by any particular theory, Applicant envisioned type III-E effectors as a natural fusion of modular proteins with interchangeable orthologous domains. The unique assembly of, essentially, individual protein domains into a single synthetic protein, provided evidence of a naturally occurring fusion system. Without wishing to be bound by any particular theory Applicant hypothesized that interchanging domains between orthologs may enhance certain functions, such as improving catalytic activity and experimentally test this in mammalian cells.

Applicant identified an architecture of a type III-E-like protein composed of Cas7-like domains and a Cas1-like domain. Applicant used this novel composition to engineer recently characterized type III-E effectors into synthetic Cas effectors (term “Cas7-S”). Applicant demonstrates a likely novel function of Cas1 in RNA-targeting and provide a method for designing synthetic RNA-targeting Cas effectors.

### Results

### Identification of a Type III-E-Like Architecture Containing a Cas1 Domain

To begin engineering type III-E effectors, Applicant initially conducted a bioinformatic search and alignment to create a domain library (FIG. 14) and identify any variants with possible novel architectures.

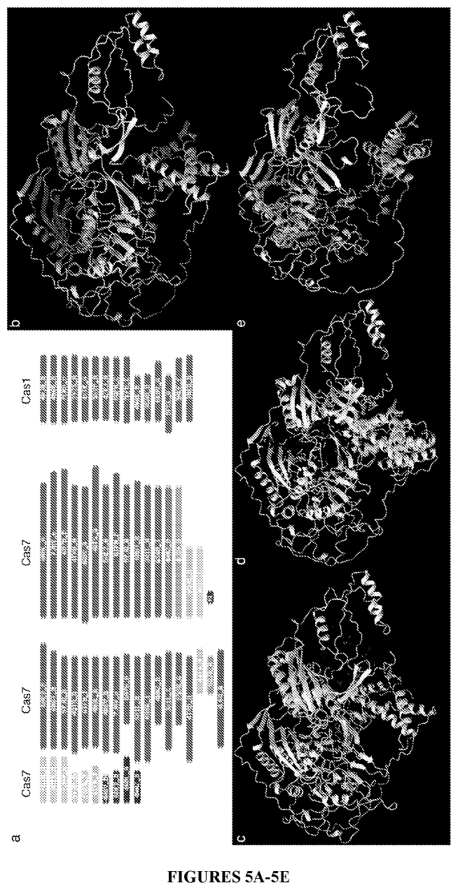

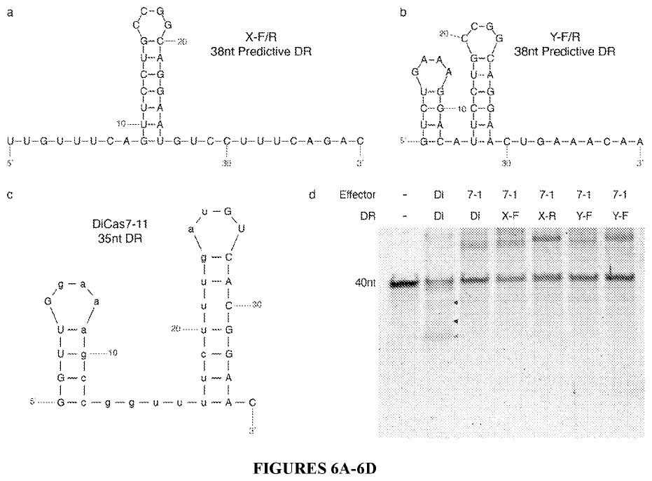

Applicant obtained an unusual blast hit for a protein denoted as a Cas1 nuclease (MBU1487208.1). Applicant analyzed the domain architecture of this protein using HHPred and found the protein to be composed of three Cas7-like domains and one Cas1 nuclease domain (FIGS. 5A-5E). For simplicity, Applicant terms this protein Cas7-1. Applicant next searched for possible crRNAs associated with Cas7-1 and obtained a 38 nt consensus direct repeat (“DR”). Applicant obtained secondary structure predicted folds of the forward and reverse directions of the consensus DR (FIGS. 6A-6B), where Applicant found the reverse direction to contain a similar stem-loop structure to the DR of DiCas7-11 (FIG. 6B-6C). Applicant biochemically tested Cas7-1 targeted cleavage activity with 5′ and 3′ orientations of the 2 DR sequences (Table 5) and observed no cleavage activity (FIG. 6D).

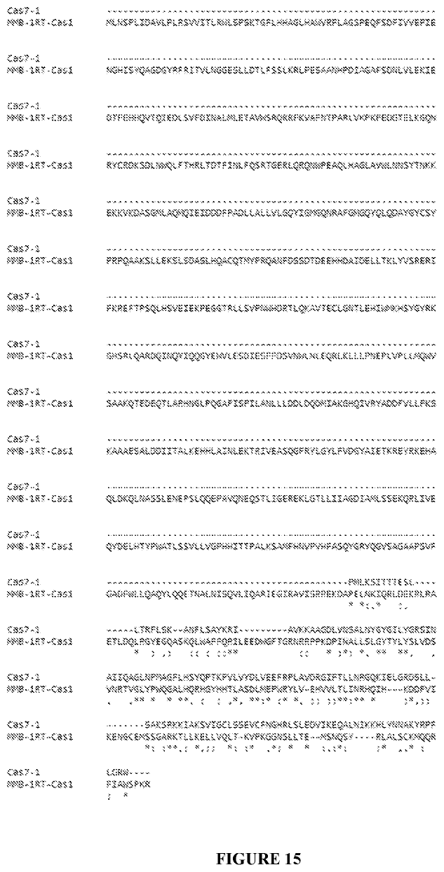

Although Cas7-1 demonstrated no enzymatic activity, the protein aligns well to all known type III-E effectors and through further analysis Applicant found interesting features of the protein. Cas7-1 appears to lack an INS domain—a domain found in all known type III-E effectors (3) (FIG. 1A). Sequences of Type III-effectors referenced in FIG. 1 are included in Table 1. Applicant also found Cas7-1 contains a conserved catalytic residue that aligns to all known Cas7.3 domains, as well as zinc-finger motifs unique to type III-E effectors (4) (FIGS. 1B-1E). Lastly, the Cas1 domain in Cas7-1 aligns to a Reverse Transcriptase-Cas1 (RT-Cas1) fusion protein commonly associated with type III systems of all characterized subtypes (III-A, III-B, III-C, and III-D) (FIG. 1F, FIG. 15). In these systems, the active Cas1 domain is required for adaptation of new spacers from interfering RNA (16). Without wishing to be bound by any particular theory, Applicant hypothesized that Cas1 may function by orienting the target substrate RNA for cleavage. However, from the alignments and structural prediction (FIG. 1G), it appears Cas7-1 is missing a portion of the N-terminus, including a CRISPR RNA (crRNA) processing domain and a secondary catalytic residue, which likely explains the lack of activity.

Engineering Synthetic Cas Effectors with Varying Architectures

Applicant suspected the missing domains of Cas7-1 would abolish crRNA recognition and processing and, partially, cleavage activity. Therefore, Applicant reasoned activity of the Cas7-1 protein could be rescued through the incorporation of Cas7.1 and Cas7.2 domains from D. ishimotonii Cas7-11 (DiCas7-11, SEQ ID NO: 6). Without wishing to be bound by any particular theory, Applicant also hypothesized that these Cas7-S effectors could be generated using a method termed Orthologous Domain Substitution (ODS), where aligned domains from type III-E effectors are substituted between orthologs. To rescue Cas7-1 activity and test the ODS design method, Applicant designed Cas7-S (designated SynCas in Table 2) effectors with different domain and linker substitutions, while simultaneously varying the overall architecture based on Cas7-1 or typical Cas7-11 proteins (FIG. 2A, FIG. 7, FIG. 8A, FIG. 17, Table 1, Table 2). Applicant obtained predicted structures from AlphaFold (17-19) to align the models against the solved structure of DiCas7-11 and use them for downstream engineering.

Since no previous study has utilized a Cas1 domain in programmable RNA-targeting and Applicant suspected the Cas1 domain would operate to orient the target substrate, Applicant focused on Cas7-S variants that were either Cas1-based or Cas11-based (FIG. 7). Sequence identifiers corresponding to the sequences in FIG. 7 are in Table 2. Applicant divided the groups by architecture, aiming to determine if the Cas7-1 orientation is functional or the typical Cas7-11 architecture reigned supreme. Applicant tested Cas7-S variants in HEK293T cells using an EGFP reporter assay and analyzed samples via qPCR. Applicant transiently transfected SynCas constructs with crRNAs containing direct repeats specific to DiCas7-11 and spacers corresponding to EGFP and assessed targeted knockdown via RT-qPCR. crRNAs used in assessment contained DRs respective to DiCas7-11 and spacer sequences complementary to EGFP (Table 3). Although Applicant observed significant RNA knockdown with the Cas7-1 architecture (Cas7-S1-S9), most variants exhibited high variability and the best variant (Cas7-S7) includes the large INS domain (FIG. 2B). In the Cas7-11 architecture group (Cas7-S10-S16), Applicant observed consistent RNA knockdown comparable to DiCas7-11 from multiple variants including one Cas1-based variant (Cas7-S14) (FIG. 2B). Applicant identified Cas7-S10-12 (where Cas7-S12 is nearly identical to Cas7-11s (4)) as the best Cas11-based effectors and Cas7-S14 as the best Cas1-based effector.

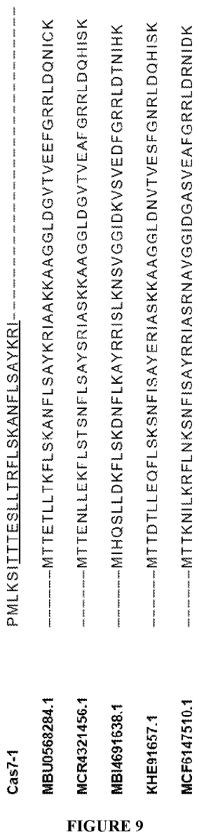

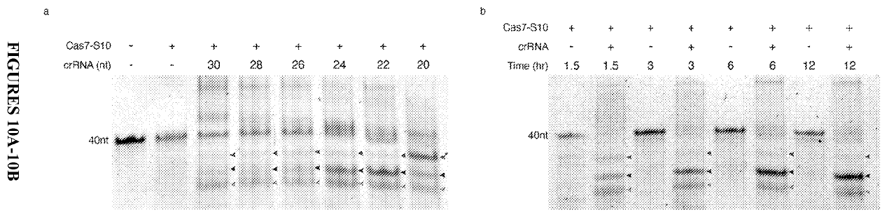

Without wishing to be bound by any particular theory, due to the natural orientation of the Cas1 domain on the C-terminus of Cas7-1, Applicant suspected the N-terminal region of the domain would be relatively unstructured and highly flexible in order to help orient the Cas1 domain underneath target substrates. Applicant identified a 29 amino acid (AA) stretch that aligned specifically to the N-terminus of the RT portion of RT-Cas1 fusion proteins (FIG. 9). Using the predicted structure of Cas7-S14, Applicant generated two truncated versions of the Cas1 domain (FIG. 2C) and tested these truncations with Cas7-S14, Cas7-S15, and Cas7-S16. Aside from Cas7-S15B, Applicant found both truncations generally improved knockdown activity for the Cas7-S variants and observed that complete deletion of the linker region consistently improved activity likely due to increased stabilization of the Cas1 domain (FIG. 2D). Together these findings demonstrate RNA-targeting type III-E effectors are highly modular and Cas1 can be used in RNA-targeting applications.

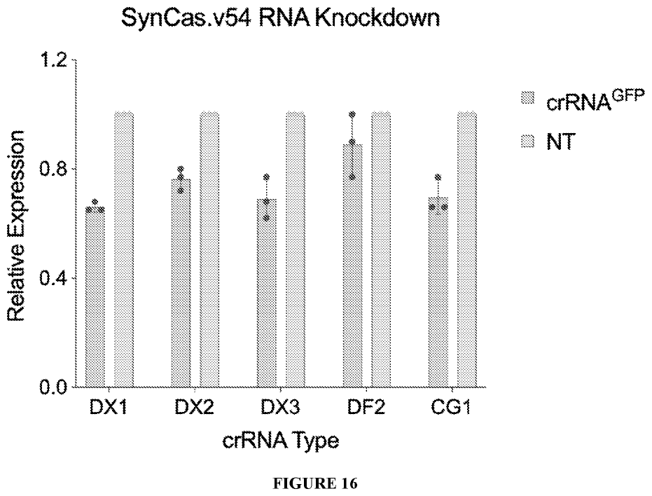

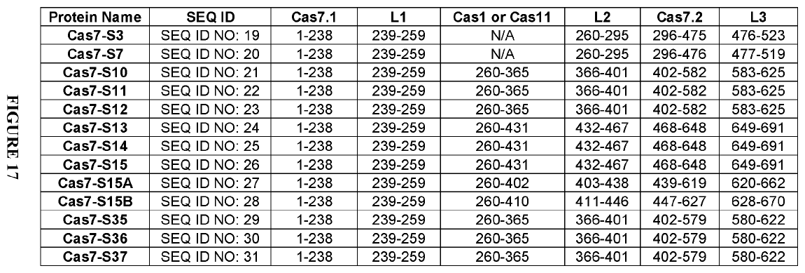

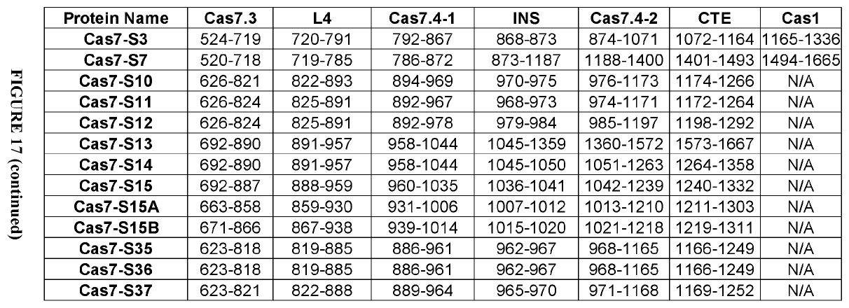

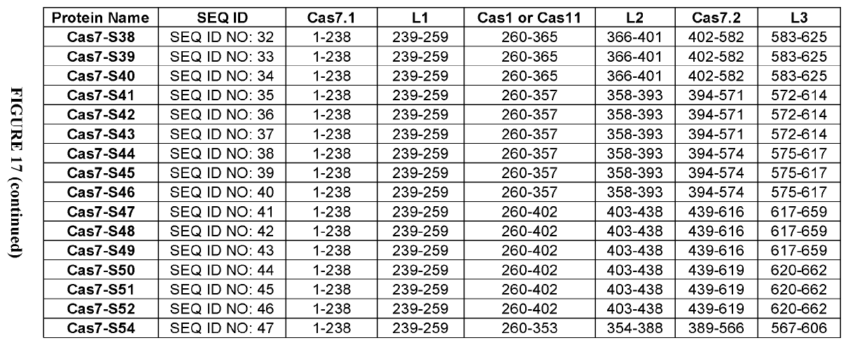

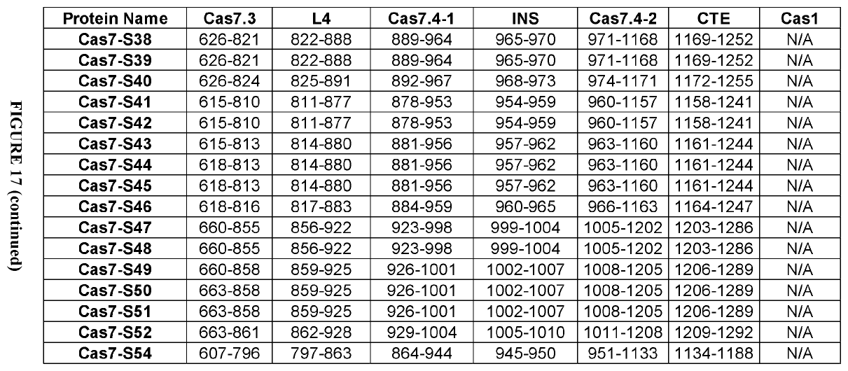

Applicant identified SynCas.v54 as the most compact SynCas effector that demonstrates programmable RNA-targeting. SynCas.v.54 is 1188 amino acids in length and knocked down gene expression up to 34% compared to the control (FIG. 16). Applicant demonstrates the RNA-targeting activity in HEK293 cells and analyzed the abundance of RNA using qPCR. All samples were compared with negative controls (non-targeting spacers). SynCas.v54 is a combination of domains and linkers from 8 different type III-E effectors. SynCas.v54 domain derivatives: Cas7.1=DiCas7-11; Linker 1=DiCas7-11; Cas11=HsmCas7-11; Linker 2=HsmCas7-11; Cas7.2=HvsCas7-11; Linker 3=SstCas7-11; Cas7.3=OmCas7-11; Linker 4=DiCas7-11; Cas7.4 (1&2)=CsbCas7-11; 6AAINS=Cas7-1; CTE=CbfCas7-11. Cas7.1 (1&2) indicates that the INS is in the middle of the Cas7.4 domain, breaking it up into two sections (see e.g. FIG. 8 for exemplary proteins showing the placement of the INS domain).

Programmable Cleavage of ssRNA with Cas7-S Effectors in Vitro

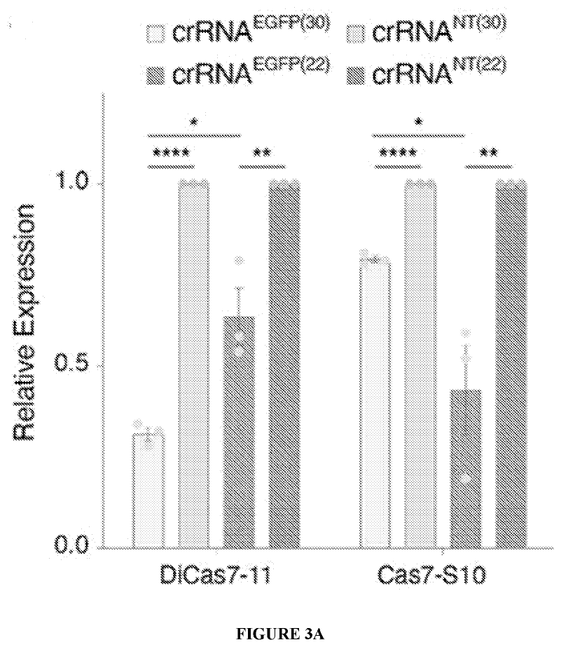

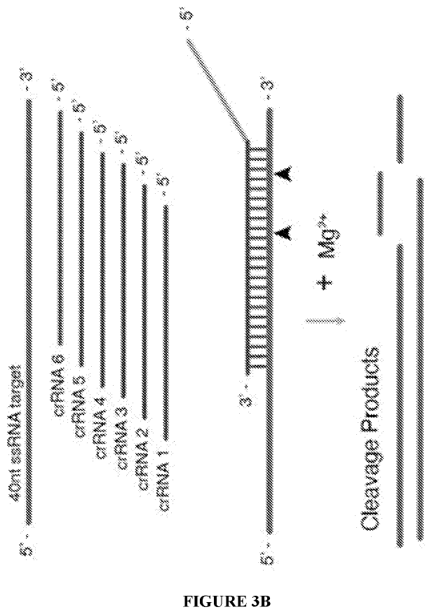

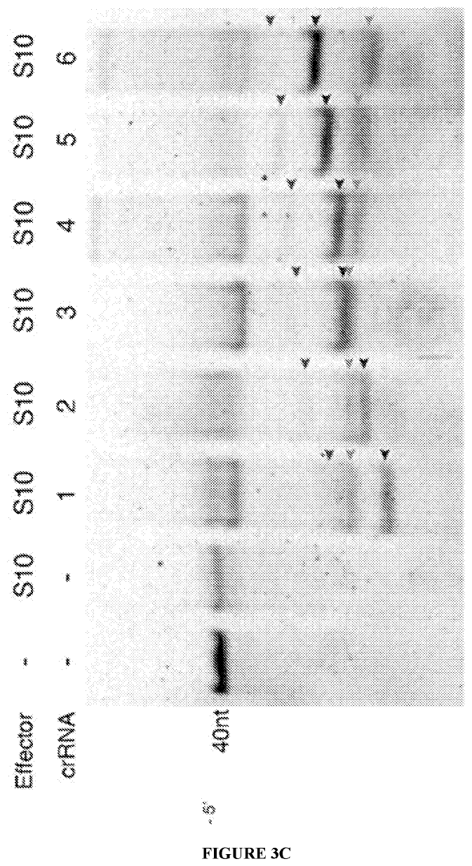

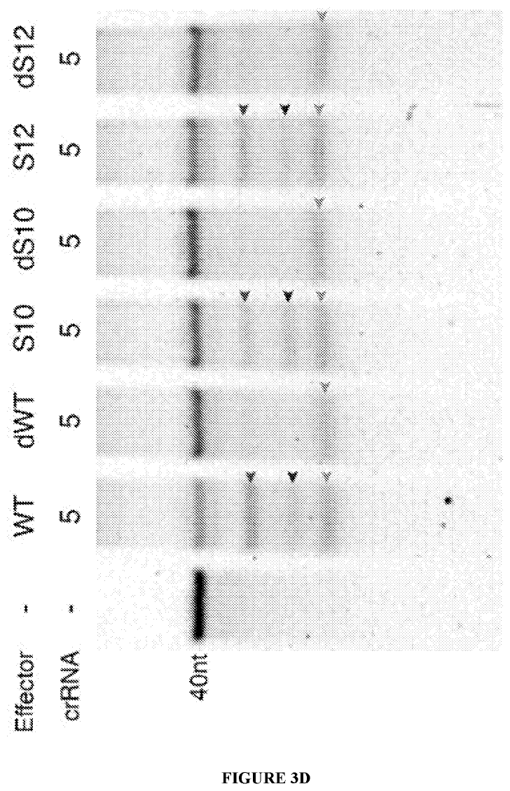

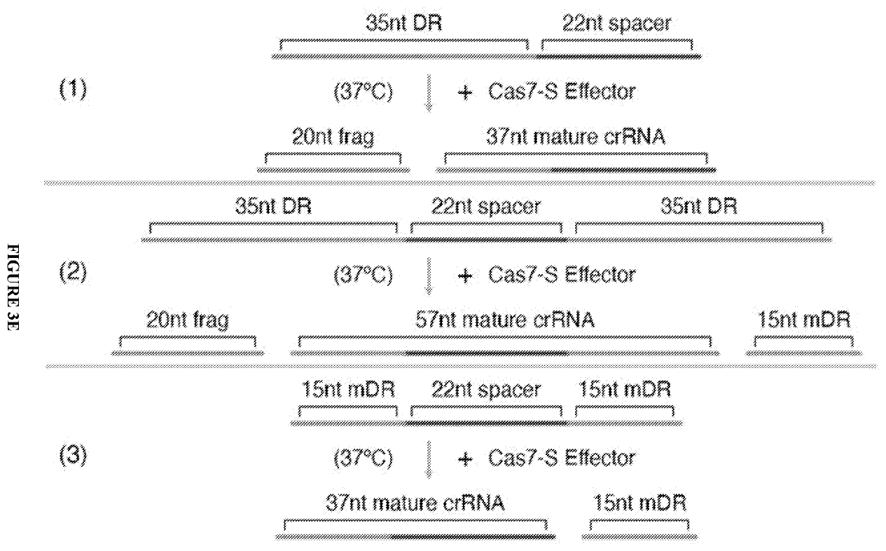

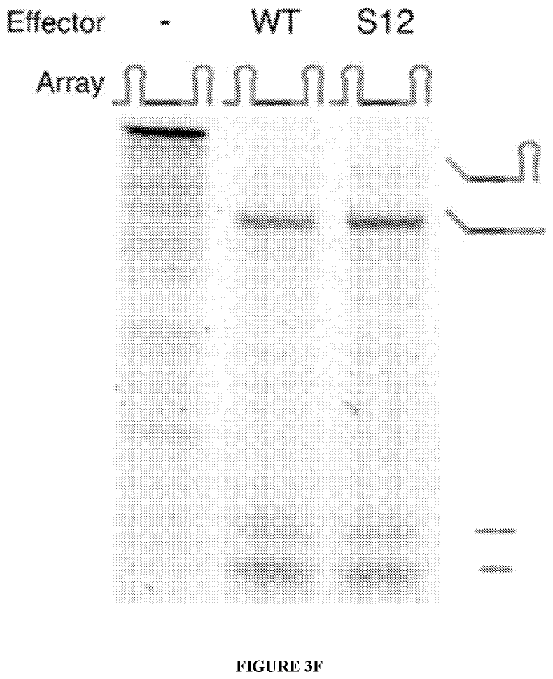

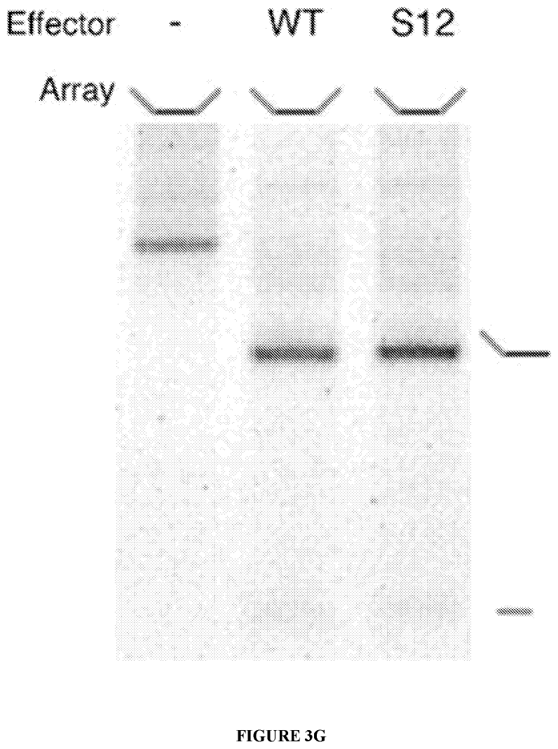

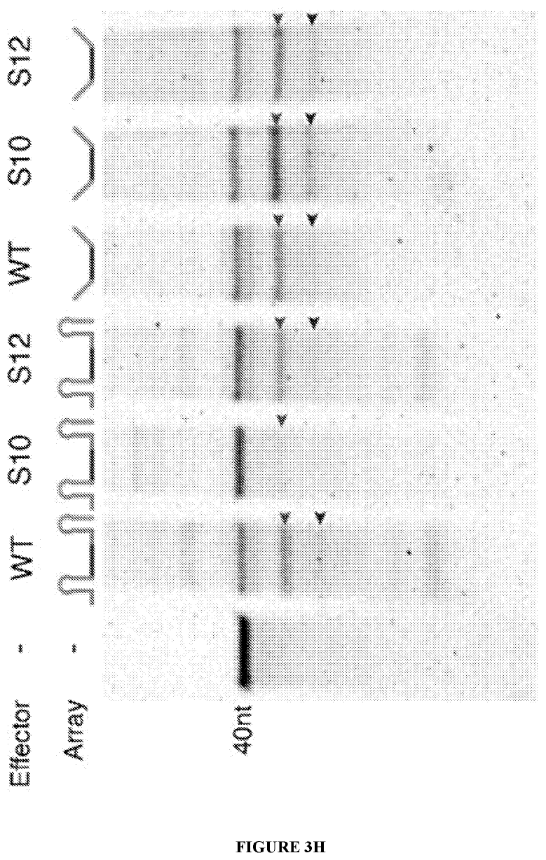

With evidence of RNA knockdown in cell culture, Applicant next wanted to characterize the mechanism of cleavage of the Cas7-S effectors. Applicant focused on Cas7-S10 to demonstrate that substitution of enzymatic domains permits retention of enzymatic activity and that RNA-targeting is unperturbed. Since Cas7-S10 contains the Cas7.1 domain deriving from DiCas7-11, all crRNAs were designed with DRs respective to DiCas7-11 (Table 3). Applicant ran an initial assay truncating the spacer of the crRNA and observed cleavage for spacers between 20-30 nt in length. Interestingly, Applicant noticed a substantial increase in both crRNA processing and target cleavage as spacers were shortened, with the best cleavage activity observed for 22-24 nt spacers (FIG. 10A). This increase in cleavage activity due to spacer truncation was further replicated with Cas7-S10 in cell culture via qPCR analysis (FIG. 3A). Applicant also extended incubation time of the cleavage reaction from 1.5 hours up to 6 hours. Applicant found 1.5 hours of incubation is sufficient to obtain noticeable cleavage of the target substrate, however complete cleavage of the target substrate was observed after 6 hours of incubation (FIG. 10B). This suggests Cas7-S10, like type III-E effectors, accomplish target cleavage slowly.

To visualize both crRNA processing and target cleavage, Applicant designed several crRNAs across a 40 nt ssRNA target and incubated the Cas7-S effectors with unprocessed crRNAs (FIG. 3B). Tiling the crRNAs across the ssRNA target and incubating for 6 hours to clearly visualize the complete cleavage product, Applicant observed stepwise cleavage products demonstrating activity of both enzymatic domains (FIG. 3C). Cleavage activity was completely abolished through mutagenesis of both active sites for DiCas7-11, Cas7-S10, and Cas7-S12 (D429A/D654A), while crRNA processing was unaffected (FIG. 3D).

Applicant investigated the crRNA processing activity further by introducing a full array structure containing the same spacer as crRNA-5 (FIG. 3E, Table 3). Differing from other reports (2, 3), Applicant found that neither Cas7-S12, nor wild-type (WT) DiCas7-11, could completely process an array structure into a mature 37 nt crRNA, but instead yielded an incompletely processed 57 nt product (FIG. 3F). Applicant found only the Cas7.1 domain processed the DR producing a product where a 15 nt mature DR (mDR) is upstream of the spacer and a 20 nt DR fragment (DRf) remains at the 3′ end (FIG. 3E). This suggests that type III-E effectors do not cleave off the DRf downstream of the spacer when processing arrays. However, by modifying the array structure to incorporate mDRs surrounding the spacer sequence, Applicant was able to obtain a crRNA without excess nucleotides on the 3′ end (FIG. 3G). This array design is extremely important for targeting and multiplexing applications with Cas7-S effectors, as Applicant found Cas7-S10, which contains Cas7.3/Cas7.4/CTE domains from Cas7-1, RNA cleavage activity is partially inhibited when the DRf is present (FIG. 3H). Together, these results suggest that Cas7-S effectors accomplish RNA cleavage like other Cas7-11 proteins and type III-E CRISPR-Cas systems use a particular crRNA structure and processing mechanism similar to previous reports of type I and type IV CRISPR-Cas systems (20).

### Generation of Compact Cas7-S Effectors for RNA-Targeting

Cas7-11 proteins are the largest single-effector Cas protein family discovered (2, 3). In therapeutic applications, this large size is a major obstacle for delivery modalities and efforts have quickly been made to reduce the size of these enzymes (4). Without wishing to be bound by any particular theory, Applicant hypothesized the ODS design method could be applied to engineer compact effectors. To test this, Applicant selected three effectors (DiCas7-11, HvsCas7-11, and Cas7-1) to generate a set of 36 compact Cas7-S effectors and further refine our ODS design method for systematic interrogation of domain arrangement (FIG. 4A, FIG. 11, FIG. 8B). Applicant included Cas7.4 from Cas7.1 in all compact Cas7-S effectors and also maintained all linkers from DiCas7-11, except when HvsCas7.1 and HvsCas11 were encoded together. The compact effectors varied in size from 1233-1292AA in length and are far smaller than the average size of Cas7-11 proteins (>1600AA) (Table 2).

To assess the RNA knockdown capabilities of the compact Cas7-S effectors, Applicant analyzed these proteins using the previously described reporter assay in HEK293T cells, and quantified knockdown using qPCR and flow cytometry (FIG. 4A). Applicant found that Cas7-S effectors containing the DiCas7.1 domain resulted in consistent EGFP knockdown comparable to DiCas7-11, while effectors containing the HvsCas7.1 domain demonstrated little-to-no knockdown activity (FIG. 4B). Within the DiCas7.1 groups, Applicant also found that either DiCas11- or HvsCas11-based variants greatly outperformed Cas7-S effectors with a Cas1 domain, contrary to prior analysis of the first 16 variants, where Cas7-S14A demonstrated comparable knockdown capabilities to DiCas7-11. Of the Cas11-based effectors, Cas7-S35, Cas7-S39, and Cas7-S41 demonstrated knockdown comparable to DiCas7-11, where Cas7-S41 is the compact (1241AA) and represents the one of the smallest type III-E effector developed by Applicant (FIG. 8C). Interestingly, composing the Cas7-S effector of HvsCas7.2 and HvsCas7.3 resulted in high RNA knockdown regardless of the Cas11/Cas1 domain used, which was not observed for any other Cas7.2-Cas7.3 combination (FIG. 4B, FIG. 8D).

Applicant further analyzed the Cas7-S effectors to gauge their ability to reduce the expression of target proteins. Using flow cytometry, Applicant observed knockdown of EGFP and mCherry with Cas7-S41 and Cas7-S47 at comparable levels to DiCas7-11 for both targets tested (FIG. 4C). This demonstrates that Cas7-S effectors can elicit phenotypic effects on a target of interest regardless of domain composition and at levels similar to wild-type type III-E effectors. Furthermore, Applicant demonstrates Cas7-S effectors are capable of knocking down endogenous genes as effectively as DiCas7-11 (FIG. 4D). Applicant also shows Cas7-S effectors do not possess the collateral cleavage activity found in other CRISPR-based RNA-targeting systems, such as Cas13. Applicant accomplished this by running SENSR reactions (21) with Cas7-S effectors and comparing them to RfxCas13d. From the fluorescence assays, Applicant found no evidence of collateral cleavage activity with any effectors composed of type III-E domains (FIG. 4E), consistent with previous reports (3).

Here Applicant demonstrate that the Cas7-11 proteins are modular and can be engineered into compact, efficient, and highly specific Cas7-S effectors for RNA-targeting applications.

### DISCUSSION AND CONCLUSION

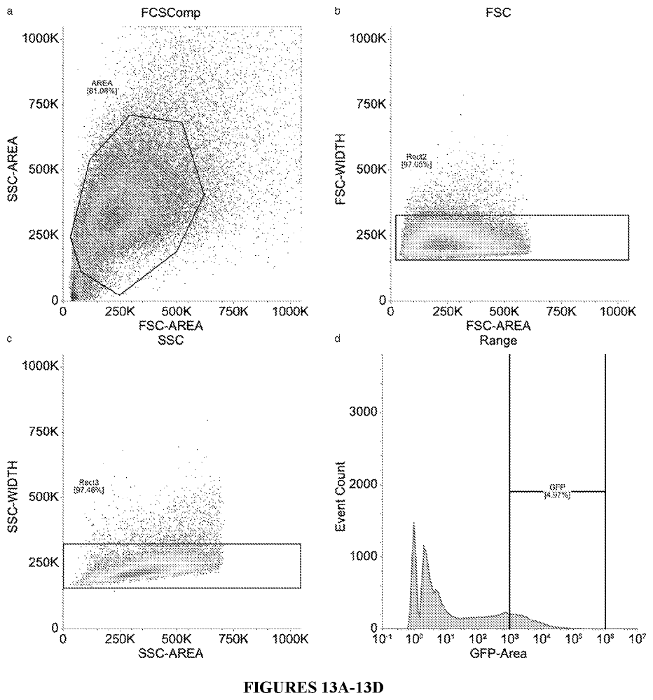

Applicant observed clear evidence of RNA-targeting in vitro and in mammalian cells. However, the level of activity exhibited by Cas7-S effectors in mammalian cells did not generally outperform wildtype DiCas7-11. This activity gap suggests further refinement is required to improve engineering of Cas7-S effectors. This refinement can be accomplished with larger libraries of Cas7-S effectors, incorporating machine learning to guide library design, or employing other downstream protein engineering methods, such as direct evolution (24, 25). RNA cleavage by Cas7-11 enzymes is inherently slow due to the evolution as a caspase system for antiviral defense, which operates more effectively with longer target binding (6, 11, 12). This slow turnover is exemplified when comparing RNA knockdown by DiCas7-11 against RfxCas13d (FIG. 12). Therefore, future studies should focus on increasing the rate of catalysis to enhance activity, while also maintaining high target specificity.

### Experimental Methods

### Bioinformatics

Cas7-1 was identified through BLAST queries of type III-E CRISPR-Cas effectors (MBU1487208.1). The amino acid sequence of Cas7-1 was submitted to HHPred for individual domain assignment (toolkit.tuebingen.mpg.de/tools/hhpred). Assumed DRs of Cas7-1 were identified by submitting metagenomic data related to Cas7-1 to CRISPR-Cas++ (crisprcas.i2bc.paris-saclay.fr/). DR secondary structure prediction was obtained using UNAFold (unafold.org/). Amino acid sequence alignments were performed using the Clustal Omega.

### Structure Guided Rational Design

SynCas proteins are designed based on naturally occurring amino acid sequences found in nature, but that does not mean their structural arrangements are identical or completely reflected by structural models, like those obtained from AlphaFold. Obtaining the structures of these proteins would permit structural interrogation providing an avenue to selectively engineer the proteins for improved activity, such as faster rates of cleavage, repeat activity (on/off rates), or better expression. This mode of engineering will also allow for selective reduction in requirements in amino acids, that will likely permit improved compactness.

This method can also be compounded with another method such as Deep Mutational Scanning (DMS), where all 20 amino acids are individually substituted into each position along the SynCas proteins. The best mutants that are obtained from this scan can be used with and by Structure-guided Rational Design.

### Design and Cloning of Constructs

Plasmids for expression of effectors in HEK293T cells were designed with a CMV promoter and bovine growth hormone (bGH) terminator. Effector plasmids were generated using standard gibson assembly methods. crRNA plasmids were designed for expression driven by a U6 promoter and terminator. crRNA expression plasmids were generated using standard golden gate assembly methods. Select plasmids are available at addgene.org/(Table 4).

### Mammalian Cell Culture

HEK293T cell lines were obtained from the American Type Culture Collection (ATCC CRL-3216) and used in all mammalian cell experiments. HEK293T cells were cultured in Dulbecco's Modified Eagle Medium (DMEM) (Thermo Fisher Scientific 11995073) supplemented with 10% Fetal Bovine Serum (FBS) (Corning 35-011-CV) and 1% penicillin-streptomycin (Thermo Fisher Scientific 15070063). Transient transfections were carried out with Lipofectamine 3000 (Thermo Fisher Scientific L3000001) according to the manufacturer's protocol.

### RNA-Knockdown Assay

To assess RNA knockdown, HEK293T cells were co-transfected with 20 ng of either the EGFP or mCherry reporter plasmid, 600 ng of a respective Cas effector plasmid, and 300 ng of crRNA plasmid. Cells were seeded in 48-wells 18-20 hours prior to transfection at seeding densities of 32,000 cells per well. Following transfection, cells were incubated for 48 hours at 37° C., 5% CO₂. After 48 hours, cells were removed from the plate with 250 μL of RNAprotect Cell Reagent (Qiagen 76104) and stored at −20° C. for at least 24 hours prior to extraction. For each effector tested, four biological replicates were performed and a non-target crRNA was used as a control.

Total RNA Collection and qPCR

To measure the reduction of endogenous or reporter genes, the transfected cells, preserved in RNAprotect, were lysed using QIAshredder (Qiagen 79656). Total RNA was then extracted using the RNeasy Mini Kit (Qiagen 74106) according to the manufacturer's protocol. Following extraction, the total RNA was treated with DNase (Thermo Fisher Scientific AM1907) to remove any remaining DNA. Sample RNA concentration was analyzed using a Nanodrop OneC UV-vis spectrophotometer (Thermo Fisher Scientific NDONEC-W). Approximately 0.5 μg of total RNA was used to synthesize cDNA with a RevertAid First Strand cDNA Synthesis Kit (Thermo Fisher Scientific K1622). cDNA was diluted (1:1000) for qPCR analysis. qPCR was performed with SYBR green (qPCRBIO SyGreen Blue Mix Separate-ROX 17-507B, Genesee Scientific), using 4 μl of dilute cDNA in each 20 μl reaction containing a final primer concentration of 200 nM and 10 μl of SYBR green buffer solution. Three technical replicates were performed for each reaction. Pipetting was performed using EpMotion® 5075 (Eppendorf). The following qPCR protocol was used on the LightCycler® 96 (Roche): 3 min of activation phase at 95° C., 40 cycles of 5 sec at 95° C., and 30 sec at 60° C. The relative expression levels of EGFP were calculated using the manufacturer's software and the delta-delta Ct method (2-ΔΔCt), with the GAPDH gene serving as a reference (26). To assess differences in EGFP expression between the non-target and targeting crRNA conditions for each effector, statistical analysis was performed using GraphPad Prism 10. Specifically, data were analyzed using an unpaired t-test.

### Flow Cytometry

Fluorescence knockdown was assessed using flow cytometry on the BioRad S3e cell sorter system. To prepare cells for analysis, transfected HEK293T cells were washed with PBS pH 7.4 (Gibco 10010031), all media was removed, then 40 μL of Accumax (VWR AM105) was added to the well. 230 μL of cold PBS pH 7.4 was added to the wells and cells were resuspended and transferred to a cell strainer (Corning 352235). Cells were then assessed on the S3e cell sorter with a count of 80,000 cells per sample. Flow cytometry data were analyzed using Floreada.io (floreada.io/) (FIG. 13).

### Recombinant Protein Expression and Purification

Plasmids used for protein expression in E. coli were generated by subcloning ORFs from plasmids used for mammalian cell culture expression, along with an N-terminal 6×His-SUMO into a pET28a vector backbone. Plasmids were then transformed into Rosetta™ 2 (DE3)pLysS Competent Cells (Novagen). Single colonies were used to inoculate a 40 mL LB overnight starter culture. The starter cultures were then transferred to a 1 L LB culture in 4 L baffled flasks and grown at 37° C. with shaking (200 rpm) until OD600 reached ~1.0. Cultures were then brought to 4° C. before protein expression was induced with 0.2 mM isopropyl β-D-thiogalactopyranoside (IPTG) and grown for 18-20 hours at 18° C. Cells were then pelleted and resuspended in a lysis buffer containing 50 mM Tris-HCl pH 8.0, 300 mM NaCl, 10 mM imidazole, 5% glycerol (v/v), and 3 mM β-mercaptoethanol supplemented with Protease Inhibitor Cocktail (Sigma P2714), 5 mM PMSF, and 2.5 U/mL salt active nuclease (Sigma SRE0015). Cells were lysed via sonication and clarified by centrifugation. His-tagged protein was bound to 2 mL HisPur™ Ni-NTA Resin (Thermo 88221) equilibrated with lysis buffer in a glass Econo-Column® Chromatography Columns (Bio-Rad) by flowing through the clarified lysate. A wash step was then performed by adding 12 column volume (CV) of a wash buffer containing 50 mM Tris-HCl pH 8.0, 500 mM NaCl, 30 mM imidazole, and 5% glycerol followed by elution with 7 CV elution buffer containing 50 mM Tris-HCl pH 8.0, 300 mM NaCl, 300 mM imidazole, and 5% glycerol. The 6×His-SUMO tag was cleaved off by dialyzing the eluate overnight at 4° C. with ~0.6 mg of in-house produced 6×His-tagged Ulp1 (Yeast SUMO Protease) in a dialysis buffer containing 20 mM HEPES-NaOH pH 7.5, 250 mM NaCl, 5% glycerol, and 1 mM DTT. Dialyzed sample was then flowed over the same Ni-NTA column equilibrated in a buffer containing 20 mM HEPES-NaOH pH 7.5, 250 mM NaCl, 25 mM imidazole, 5% glycerol, and 1 mM DTT to remove additional E. coli impurities, Ulp1, and the 6×His-SUMO tag. Cation exchange chromatography was performed with 2×1 mL HiTrap Heparin HP Column (Cytiva) with a NaCl gradient from 200-1000 mM NaCl followed by gel filtration chromatography with a HiLoad 16/600 Superdex 200 column equilibrated in 20 mM HEPES-NaOH pH 7.5, 600 mM NaCl, 5% glycerol (v/v), 2 mM DTT on an ÄKTA Pure (Cytiva). Proteins were then concentrated to ~10 μM and stored in small aliquots at −80° C. for future use.

Nucleic Acid Target and crRNA Preparation

Synthetic ssRNA templates and the full DiCas7-11 crRNA array were ordered custom from IDT. All other crRNAs were produced in house. To generate crRNAs, a dsDNA template with a T7 promoter incorporated was produced through templateless PCR and purified with MinElute PCR Purification Kit (Qiagen 28004). dsDNA templates were converted to ssRNA through in vitro transcription using MEGAScript™ T7 Transcription Kit (Invitrogen AM1334) and purified with MEGAClear™ Transcription Clean-Up Kit (Invitrogen AM1908).

### Nuclease Assays

Nuclease assays were run in 10 μL reactions with the following final concentrations: 500 nM protein, 200 nM crRNA, 240 nM probe, 40 U RNase Inhibitor, and 10 mM MgCl₂. Reactions were supplemented with a custom 10× reaction buffer (200 mM HEPES (pH 7.5), 600 mM NaCl) and incubated at 37° C. Times were varied based on experiment. Upon completion, reactions were denatured with 2×RNA dye (NEB B0363S) at 95° C. for 10 min. 10 μl of dyed samples were loaded onto a prerun (200V for 60 min) 15% TBE-Urea PAGE gel (BioRad 4566055) and resolved at 200V for 35 min. Gels were then stained with SYBR Gold (Invitrogen S11494) and incubated for 10 min at room temperature on a shaker and washed in 1×TBE for 10 min on a shaker. Stained gels were imaged using Enduro™ GDS (Labnet). SENSR assays were conducted following a previously described protocod (21) with slight alterations: 100 nM of RfxCas13d incubated in reaction and 200 nM of DiCas7-11 or Cas7-S effector incubated in reaction.

### Example 2—RNA Therapeutics

Compact SynCas effectors, like SynCas.v54 and the others presented herein, can be packaged into AAV vectors and delivered to the central nervous system. The RNA-targeting modality of SynCas effectors makes them ideal for therapeutic treatment against microsatellite repeat expansion (MRE) disorders such as Huntington's disease, ALS, myotonic dystrophy, etc. The compact size, compounded with the lack of collateral cleavage seen in other CRISPR-Cas RNA-targeting systems like Cas13, make SynCas a valuable tool for RNA-therapeutic development. SynCas vectors can be delivered to tissues including but not limited to the heart, brain (and select lobes/regions), muscle, kidneys, and ears.

### Example 3—Agricultural Applications

SynCas can also be utilized as a viable defense mechanism against viruses. Honeybees colonies are often infested with mites known as Varroa destructor. These mites transmit a ssRNA virus, Deformed Wing Virus (DWV), which can negatively impact honey production, pollination, or the overall health of bee colonies, all cumulatively leading to economic ramifications. Genetically encoding a SynCas effector and crRNA array in a honeybee colony would make it possible for a colony to be resistant or immune to DWV and can ultimately prevent severe events like colony collapse.

Plants are also exposed to numerous viruses, many of which are ssRNA viruses. Just like with honeybees and DWV, SynCas can be encoded to protect plants against invading ssRNA viruses that significantly impact commercial production of foods, such as rice, maize, soy, or potatoes. The RNA-targeting nature of these enzymes also helps ensure DNA damage and deleterious mutations do not occur.

### Tables

TABLE 1
Sequences of Type-III effectors used in the sequence alignments and/or
synthetic proteins. Sequences which have a 100%
match to a sequence in NCBI are indicated with
the NCBI accession number. The domain information
for each effector protein in Table 1
can be found in FIG. 14.

| Name | | |
|---|---|---|
| (SEQ ID NO:) | | |
| [NCBI Reference | | Sequence |
| Number] | Sequence | Length (AA) |
| Cas7-1 | MAKLLCLDLEVGPEGIVFPFVMRYRGQGEKLPS | 933 |
| (SEQ ID NO: 1) | ELTDVIHLWKEEMAFLGGSASTGKGRFSLEPLDI | |
| [MBU1487208.1] | FQWDLTDPGSLKDYIECSGLRRPEEELEEWSEA | |
| | LQGLNPFSLPDTSDKYSPQWTKVGYEIKIQSPLL | |
| | SNDPIAALFDTANHDAVAYKKRVWENGRIIERH | |
| | TFKGESIRGVLRTALGREYGLFELKHEDCPCSLC | |
| | TIFGNEHEAGKIRFEDLEIEDGVAEKHFDHVAID | |
| | RFTGGAVDKKKFDDFSLAGSPGKPIKLKGCFWL | |
| | RADIFSDEKKDEKKKIINALCDVRDGLYPLGGK | |
| | GGVGYGWVKELRITDGPDDFKLPEPKCKEIPPD | |
| | GTPSDYPFPQLKPLQTEKDKVYSPHYFLKPAEK | |
| | KVDRNWEKRLIGHERFDKDLLTGKIECTLKTLS | |
| | PLIIPDSEVVTENGNGHKSYQFFRLNNEIIIPGSEI | |
| | RGMISSVYEALTNSCFRVVDDIIPVVISRSVSPYQ | |
| | IAQKLPSPDLRPCEREILEDSEIGSLDKCSEKRLF | |
| | RLHEDGLCPSCRLFGTTHYKGRVRFGFAKHEGG | |
| | EKWLMDDKTKDEYLTLPLLERPRPTWSMPSDE | |
| | DDVPGRKFYVHHNGWEKVKQDSLDNKTSKTE | |
| | NNRSVEPLDKDNLFKFDVFFENLEPWELGLLLY | |
| | SLELEEGLAHKFGMAKAFGFGSTKIDADKILLR | |
| | EAPNEWQDATNRKDDMLRKGFGELTSWFDKD | |
| | WDKVEHIDGLRSLLQIPEALSRAEVRYPDLTED | |
| | DPEKPPGYVQLSDKKEGRKYLIRPWQPWYPML | |
| | KSITTTESLLTRFLSKANFLSAYKRIAVKKAAGD | |
| | LVNSALNYGYGILYGRSINAIIQAGLNPMAGFLH | |
| | SYQPTKPVLVYDLVEEFRPLAVDRGIFTLLNRG | |
| | QKIELGRDSLLSAKSRKKIAKSVIGCLSSEVCEN | |
| | GHRLSLEDVIKEQALNIKKHLYNNAKYRPFLGR | |
| | W* | |
| CbfCas7-11 | MSKTDDKIDIKLTFLEPYRMVNWLENGLRMTD | 1573 |
| (SEQ ID NO: 2) | PRYLRGLSFARWHRNKNGKAGRPYITGTLLRSA | |
| [UJS22399.1] | VIRAAEELLSLNLGKWGKQLCCPGQFETEREMR | |
| | KNKTFLRRRPTPAWSAETKKEICTTHGSACAFC | |
| | LLLGRRLHGGKEDVNEDAPGSCRKPVGFGNLSL | |
| | PFQPTKRQIQDVCKERVLNRVDFRTGKAQDYFR | |
| | VFEIDHEDWGVYTGEITITEPRVQEMLEASLKFV | |
| | DTLCGALCRIEIVGSADETKRTTSSKEGCPASTT | |
| | TRDCSSSENDDTSPEDPVREDLKKIAHVIANAFQ | |
| | NSGNREKVHALADAIRAMRLEESSIINTLPKGKS | |
| | EKTTEQIEVNKHYLWDEIPVNDTSVRHILIEQWR | |
| | RWQSKKDDPEWWKFCDFLGECLYKEYKKLTS | |
| | GIQSRARVMGETEYYGALGMPDKVIPLLKSDKT | |
| | KEWILVGSLKAETPFFFGLETEQTEEVEHTSLRL | |
| | VMDKKGRFRIPRSVLRGALRRDMRIAFDSGCDV | |
| | KLGSPLPCDCSVCQVMRSITIKDSRSEAGKLPQI | |
| | RHRIRLNPFSGTVDEGALFDIEVAPEGVIFPFVM | |
| | RYRGEEFPPALLSVIRYWQDGKAWLGGEGATG | |
| | KGRFALAKDLKMYEWKLEDKSLHAYIDTYGHR | |
| | GNEHAIGTGQGIDGFRSGSLSDLLSDISKESFRDP | |
| | LASYHNYLDKRWIKVGYQITIGAPLLSADPIGAL | |
| | LDPNNVDAIVFEKMKLDGDQVKYLPAIKGETIR | |
| | GIVRTALGKRNNLLAKNDHDDCTCSLCAIFGNE | |
| | NETGKIRFEDLEVYDKDIAKKIDHVAIDRFTGGA | |
| | RDQMKFDTLPLIGSPERPLRLKGLFWMRRDVSP | |
| | DEKARILLAFLEIREGLYPIGGKTGSGYGWVSDL | |
| | EFDGDAPEAFKEMNSKRGKQASFKEKISFRYPS | |
| | GAPKHIQNLKATSFYYPHYFLEPGSKVIREQKMI | |
| | GHEQYYESYPSGASGEKLLSGRIICSMTTHTPLI | |
| | VPDTGVIKDPENKHATYDFFQMNNAIMIPGSEIR | |
| | GMISAVYEAMTNSCFRIFHEKQYLTRRISPEDKE | |
| | LREFIPGIVRIINGDVYIEKAEREYRLPLYDDVHII | |
| | TNYEELEYEKYIKKNPGREQKIKNAHRFNKNIA | |
| | RIAESNRNYLCSLDRAVRREILSGRKKVNFRLV | |
| | KVNDNKNPDKEAVELCKTGPLEGLVKFSGLNA | |
| | VNISNLRPGTAEEGFDAKWDMWSLNIILNRMD | |
| | VRNSQKKEYPRPALHFNHDGKEYTIPKRCERVF | |
| | VRAEAGKRAETEGSYKVPRKVQEQYQNILRDY | |
| | ESNIGHIDNTFRTLIENCGLNNGSLVYFKPDNSR | |
| | KEVVAITPVKISRKTDRLPQGDRFPHTSSDLRPC | |
| | VRDCLDTEGDIRMLENSPFKRLFHIHPEGLCPAC | |
| | QLFGTTNYRGRVRFGFASLSDGPKWFRKDEGN | |
| | ETCHITLPLLERPRPTWSMPDDTSTIPGRKFYVH | |
| | HMGYETVKKNQRTLVKTENNRTVKALDKENEF | |
| | TFEVFFENLREWELGLLLHCLELEPEMGHKLGM | |
| | GKPLGFGSVKIRIDKLQKCVVNVKDGCVLWEPE | |
| | EDKIQHYIAKGLGKLTTWFGKEWDRLEHIQGLR | |
| | SLQRLLPL | |
| CjcCas7-11 | MHTILPIHLTFLEPYRLAEWHAKADRKKNKRYL | 1812 |
| (SEQ ID NO: 3) | RGMSFAQWHKDKDGIGKPYITGTLLRSAVLNA | |
| [WP_007220849.1] | AEELISLNQGMWAKEPCCNGKFETEKDKPAVL | |
| | RKRPTIQWKTGRPAICDPEKQEKKDACPLCMLL | |
| | GRFDKAGKRHRDNKYDKHDYDIHFDNLNLITD | |
| | KKFSHPDDIASERILNRVDYTTGKAHDYFKVWE | |
| | VDDDQWWQFTGTITMHDDCSKAKGLLLASLCF | |
| | VDKLCGALCRIEVTGNNSQDENKEYAHPDTGII | |
| | TSLNLKYQNNSTIHQDAVPLSGSAHDNDEPPVH | |
| | DNDSSLDNDTITLLSMKAKEIVGAFRESGKIEKA | |
| | RTLADVIRAMRLQKPDIWEKLPKGINDKHHLW | |
| | DREVNGKKLRNILEELWRLMNKRNAWRTFCEV | |
| | LGNELYRCYKEKTGGIVLRFRTLGETEYYPEPE | |
| | KTEPCLISDNSIPITPLGGVKEWIIIGRLKAETPFY | |
| | FGVQSSFDSTQDDLDLVPDIVNTDEKLEANEQT | |
| | SFRILMDKKGRYRIPRSLIRGVLRRDLRTAFGGS | |
| | GCIVELGRMIPCDCKVCAIMRKITVMDSRSENIE | |
| | LPDIRYRIRLNPYTATVDEGALFDMEIGPEGITFP | |
| | FVFRYRGEDALPRELWSVIRYWMDGMAWLGG | |
| | SGSTGKGRFALIDIKVFEWDLCNEEGLKAYICSR | |
| | GLRGIEKEVLLENKTIAEITNLFKTEEVKFFESYS | |
| | KHIKQLCHECIINQISFLWGLRSYYEYLGPLWTE | |
| | VKYEIKIASPLLSSDTISALLNKDNIDCIAYEKRK | |
| | WENGGIKFVPTIKGETIRGIVRMAVGKRSGDLG | |
| | MDDHEDCSCTLCTIFGNEHEAGKLRFEDLEVVE | |
| | EKLPSEQNSDSNKIPFGPVQDGDGNREKECVTA | |
| | VKSYKKKLIDHVAIDRFHGGAEDKMKFNTLPL | |
| | AGSFEKPIILKGRFWIKKDIVKDYKKKIEDAMV | |
| | DIRDGLYPIGGKTGIGYGWVTDLTILNPQSGFQI | |
| | PVKKDISPEPGTYSTYPSHSTPSLNKGHIYYPHY | |
| | FLAPANTVHREQEMIGHEQFHKEQKGELLVSGK | |
| | IVCTLKTVTPLIIPDTENEDAFGLQNTYSGHKNY | |
| | QFFHINDEIMVPGSEIRGMISSVYEAITNSCFRVY | |
| | DETKYITRRLSPEKKDESNDKNKSQDDASQKIR | |
| | KGLVKKTDEGFSIIEVERYSMKTKGGTKLVDKV | |
| | YRLPLYDSEAVIASIQFEQYGEKNEKRNAKIRAA | |
| | IKRNEVIAEVARKNLIFLRSLTPEELKKVLQGEIL | |
| | VKFSLKSGKNPNDYLAELHENGTERGLIKFTGL | |
| | NMVNIKNVNEEDKDFNDTWDWEKLNIFHNAH | |
| | EKRNSLKQGYPRPVLKFIKDRVEYTIPKRCERIF | |
| | CIPVKNTIEYKVSSKVCKQYKDVLSDYEKNFGH | |
| | INKIFTTKIQKRELTDGDLVYFIPNEGADKTVQAI | |
| | MPVPLSRITDSRTLGERLPHKNLLPCVHEVNEGL | |
| | LSGILDSLDKKLLSIHPEGLCPTCRLFGTTYYKG | |
| | RVRFGFANLMNKPKWLTERENGCGGYVTLPLL | |
| | ERPRLTWSVPSDKCDVPGRKFYIHHNGWQEVL | |
| | RNNDITPKTENNRTVEPLAADNRFTFDVYFENL | |
| | REWELGLLCYCLELEPGMGHKLGMGKPMGFGS | |
| | VKIAIERLQTFTVHQDGINWKPSENEIGVYVQK | |
| | GREKLVEWFTPSAPHKNMEWNGVKHIKDLRSL | |
| | LSIPGDKPTVKYPTLNKDAEGAISDYTYERLSDT | |
| | KLLPHDKRVEYLRTPWSPWNAFVKEAEYSPSE | |
| | KSDEKGRETIRTKPKSLPSVKSIGKVKWFDEGK | |
| | GFGILIMDDGKEVSISKNSIRGNILLKKGQKVTF | |
| | HIVOGLIPKAEDIEIAK | |
| CmaCas7-11 | MLKLKVKITYFQPFRVIPWIKEDDRNSDRNYLR | 1657 |
| (SEQ ID NO: 4) | GGTFARWHKDKKDDIHGKPYITGTLLRSALFTE | |
| [KPA14974.1] | IEKIKIHHSDFIHCCNAIDRTEGKHQPSFLRKRPV | |
| | YTENKNIQACNKCPLCLIMGRGDDRGEDLKKK | |
| | KHYNGKHYQNWTVHFSNFDTQATFYWKDIVQ | |
| | KRILNRVDQTCGKAKDFFKVCEVDHIACPTLNG | |
| | IIRINDEKLSQEEISKIKQLIAVGLAQIESLAGGIC | |
| | RIDITNONHDDLIKSFFETKPSKILQPNLKESGEE | |
| | RFELAKLELLAEYLTQSFDANQKEQQLRRLADA | |
| | IRDLRKYSPDYLKDLPKGKKGGRTSIWNKKVA | |
| | DDFTLRDCLKNQKIPNELWRQFCEGLGREVYKI | |
| | SKNISNRSDAKPRLLGETEYAGLPLRKEDEKEYS | |
| | PTYQNQESLPKTKWIISGELQAITPFYIGHVNKTS | |
| | HTRSTIFLNMNGQFCIPRSTLRGALRRDLRLVFG | |
| | DSCNTPVGSRVCYCQVCQIMRCIKFEDALSDVD | |
| | SPPEVRHRIRLNCHTGVVEEGALFDMETGFQGM | |
| | IFPFRLYYESKNEIMSQHLYEVLNNWTNGQAFF | |
| | GGEAGTGFGRFKLLNNEVFLWEIDGEEEDYLQY | |
| | LFSRGYKGIETDEIKKVADPIKWKTLFTKLEIPPE | |
| | KIPLTQLNYTLTIDSPLISRDPIAAMLDNRNPDAV | |
| | MVKKTILVYEQDSSTHKNVPKEVPKYFIKSETIR | |
| | GLLRSIISRTEIKLEDGKKERIFNLDHEDCDCLQC | |
| | RLFGNVHQQGILRFEDAEITNKNVSDCCIDHVAI | |
| | DRFTGGGVEKMKFNDYPLSASPKNCLNLKGSI | |
| | WITSALKDSEKEALSKALSELKYGYASLGGLSAI | |
| | GYGRVKELTLEENDIIQLTEITESNLNSQSRLSLK | |
| | PDVKKELSNNHFYYPHYFIKPAPKEVVRESRLIS | |
| | HVQGHDTEGEFLLTGKIKCRLQTLGPLFIANND | |
| | KGDDYFELQHNNPGHLNYAFFRINDHIAIPGASI | |
| | RGMISSVFETLTHSCFRVMDDKKYLTRRVIPESE | |
| | TTQKRKSGRYQVEESDPDLFPGRVQKKGNKYKI | |
| | EKMDEIVRLPIYDNFSLVERIREYHYSEECASYV | |
| | PSVKKAIDYNRMLAQAADSNREFLYNHPEAKSI | |
| | LQGKKEVYYILHKQESKNRGKTKEINPNARYAC | |
| | LTDENTPGSRKGFIKFTGPDMVTVNKELKSKIAP | |
| | IYDPEWEKDIPDWERSNQESNHKYSFILHNEIEM | |
| | RSSQKKKYPRPVFICKKNGVEYRMQKRCERIFD | |
| | FTKEEEKDKEIVIPQKVVSQYNAILKDNKENTET | |
| | IPGLFNSKMVNKELEDGDLVYFKYKEGKVTELT | |
| | PVAISRKTDNKPMGKRFPKISINGKMKPNDSLRS | |
| | CSHTCTEDCDDCPNLCESVKDYFKPHPDGLCPA | |
| | CHLFGTTFYKSRLSFGLAWLENNAKWYISNDFQ | |
| | QKDSKKEKGGKLTLPLLERPRPTWSMPNNNAE | |
| | VPGRKFYVHHPWSVENIKNNQGNQKDISLKPDS | |
| | DAIKIKENNRTIEPLGKDNVFNFEISFNNLRDWE | |
| | LGLLLYAIELEDHLAHKLGMAKAFGMGSVKIEI | |
| | KNLLIKGSINDISKAELIKKGFKKLGIDSLEKDDL | |
| | SEYLHIKQLREILWFSDKPVGTIEYPKLENKTNS | |
| | RIPSYTDFVQEKDHETGFKNPKYQNLKSRLHILQ | |
| | NPWNAWWKNEE | |
| CsbCas7-11 | MNITVELTFFEPYRLVEWFDWDARKKSHSAMR | 1717 |
| (SEQ ID NO: 5) | GQAFAQWTWKGKGRTAGKSFITGTLVRSAVIK | |
| [KHE91659.1] | AVEELLSLNNGKWEGVPCCNGSFQTDESKGKK | |
| | PSFLRKRHTLQWQANNKNICDKEEACPFCILLG | |
| | RFDNAGKVHERNKDYDIHFSNFDLDHKQEKND | |
| | LRLVDIASGRILNRVDFDTGKAKDYFRTWEADY | |
| | ETYGTYTGRITLRNEHAKKLLLASLGFVDKLCG | |
| | ALCRIEVIKKSESPLPSDTKEQSYTKDDTVEVLS | |
| | EDHNDELRKQAEVIVEAFKQNDKLEKIRILADAI | |
| | RTLRLHGEGVIEKDELPDGKEERDKGHHLWDIK | |
| | VQGTALRTKLKELWQSNKDIGWRKFTEMLGSN | |
| | LYLIYKKETGGVSTRFRILGDTEYYSKAHDSEGS | |
| | DLFIPVTPPEGIETKEWIIVGRLKAATPFYFGVQQ | |
| | PSDSIPGKEKKSEDSLVINEHTSFNILLDKENRYR | |
| | IPRSALRGALRRDLRTAFGSGCNVSLGGQILCNC | |
| | KVCIEMRRITLKDSVSDFSEPPEIRYRIAKNPGTA | |
| | TVEDGSLFDIEVGPEGLTFPFVLRYRGHKFPEQL | |
| | SSVIRYWEENDGKNGMAWLGGLDSTGKGRFAL | |
| | KDIKIFEWDLNQKINEYIKERGMRGKEKELLEM | |
| | GESSLPDGLIPYKFFEERECLFPYKENLKPQWSE | |
| | VQYTIEVGSPLLTADTISALTEPGNRDAIAYKKR | |
| | VYNDGNNAIEPEPRFAVKSETHRGIFRTAVGRR | |
| | TGDLGKEDHEDCTCDMCIIFGNEHESSKIRFEDL | |
| | ELINGNEFEKLEKHIDHVAIDRFTGGALDKAKF | |
| | DTYPLAGSPKKPLKLKGRFWIKKGFSGDHKLLI | |
| | TTALSDIRDGLYPLGSKGGVGYGWVAGISIDDN | |
| | VPDDFKEMINKTEMPLPEEVEESNNGPINNDYV | |
| | HPGHQSPKQDHKNKNIYYPHYFLDSGSKVYREKD | |
| | IITHEEFTEELLSGKINCKLETLTPLIIPDTSDE | |
| | NGLKLQGNKPGHKNYKFFNINGELMIPGSELRG | |
| | MLRTHFEALTKSCFAIFGEDSTLSWRMNADEKD | |
| | YKIDSNSIRKMESQRNPKYRIPDELQKELRNSGN | |
| | GLFNRLYTSERRFWSDVSNKFENSIDYKREILRC | |
| | AGRPKNYKGGIIRQRKDSLMAEELKVHRLPLYD | |
| | NFDIPDSAYKANDHCRKSATCSTSRGCRERFTC | |
| | GIKVRDKNRVFLNAANNNRQYLNNIKKSNHDL | |
| | YLQYLKGEKKIRFNSKVITGSERSPIDVIAELNER | |
| | GRQTGFIKLSGLNNSNKSQGNTGTTFNSGWDRF | |
| | ELNILLDDLETRPSKSDYPRPRLLFTKDQYEYNI | |
| | TKRCERVFEIDKGNKTGYPVDDQIKKNYEDILD | |
| | SYDGIKDQEVAERFDTFTRGSKLKVGDLVYFHI | |
| | DGDNKIDSLIPVRISRKCASKTLGGKLDKALHPC | |
| | TGLSDGLCPGCHLFGTTDYKGRVKFGFAKYEN | |
| | GPEWLITRGNNPERSLTLGVLESPRPAFSIPDDES | |
| | EIPGRKFYLHHNGWRIIRQKQLEIRETVQPERNV | |
| | TTEVMDKGNVFSFDVRFENLREWELGLLLQSL | |
| | DPGKNIAHKLGKGKPYGFGSVKIKIDSLHTFKIN | |
| | SNNDKIKRVPQSDIREYINKGYQKLIEWSGNNSI | |
| | QKGNVLPQWHVIPHIDKLYKLLWVPFLNDSKLE | |
| | PDVRYPVLNEESKGYIEGSDYTYKKLGDKDNLP | |
| | YKTRVKGLTTPWSPWNPFQVIAEHEEQEVNVT | |
| | GSRPSVTDKIERDGKMV | |
| DiCas7-11 | MTTTMKISIEFLEPFRMTKWQESTRRNKNNKEF | 1601 |
| (SEQ ID NO: 6) | VRGQAFARWHRNKKDNTKGRPYITGTLLRSAV | |
| [8GS2_A] | IRSAENLLTLSDGKISEKTCCPGKFDTEDKDRLL | |
| | QLRQRSTLRWTDKNPCPDNAETYCPFCELLGRS | |
| | GNDGKKAEKKDWRFRIHFGNLSLPGKPDFDGP | |
| | KAIGSQRVLNRVDFKSGKAHDFFKAYEVDHTR | |
| | FPRFEGEITIDNKVSAEARKLLCDSLKFTDRLCG | |
| | ALCVIRFDEYTPAADSGKQTENVQAEPNANLAE | |
| | KTAEQIISILDDNKKTEYTRLLADAIRSLRRSSKL | |
| | VAGLPKDHDGKDDHYLWDIGKKKKDENSVTIR | |
| | QILTTSADTKELKNAGKWREFCEKLGEALYLKS | |
| | KDMSGGLKITRRILGDAEFHGKPDRLEKSRSVSI | |
| | GSVLKETVVCGELVAKTPFFFGAIDEDAKQTDL | |
| | QVLLTPDNKYRLPRSAVRGILRRDLQTYFDSPC | |
| | NAELGGRPCMCKTCRIMRGITVMDARSEYNAP | |
| | PEIRHRTRINPFTGTVAEGALFNMEVAPEGIVFPF | |
| | QLRYRGSEDGLPDALKTVLKWWAEGQAFMSG | |
| | AASTGKGRFRMENAKYETLDLSDENQRNDYLK | |
| | NWGWRDEKGLEELKKRLNSGLPEPGNYRDPK | |
| | WHEINVSIEMASPFINGDPIRAAVDKRGTDVVTF | |
| | VKYKAEGEEAKPVCAYKAESFRGVIRSAVARIH | |
| | MEDGVPLTELTHSDCECLLCQIFGSEYEAGKIRF | |
| | EDLVFESDPEPVTFDHVAIDRFTGGAADKKKFD | |
| | DSPLPGSPARPLMLKGSFWIRRDVLEDEEYCKA | |
| | LGKALADVNNGLYPLGGKSAIGYGQVKSLGIK | |
| | GDDKRISRLMNPAFDETDVAVPEKPKTDAEVRI | |
| | EAEKVYYPHYFVEPHKKVEREEKPCGHQKFHE | |
| | GRLTGKIRCKLITKTPLIVPDTSNDDFFRPADKE | |
| | ARKEKDEYHKSYAFFRLHKQIMIPGSELRGMVS | |
| | SVYETVINSCFRIFDETKRLSWRMDADHQNVL | |
| | QDFLPGRVTADGKHIQKFSETARVPFYDKTQKH | |
| | FDILDEQEIAGEKPVRMWVKRFIKRLSLVDPAK | |
| | HPQKKQDNKWKRRKEGIATFIEQKNGSYYFNV | |
| | VTNNGCTSFHLWHKPDNFDQEKLEGIQNGEKL | |
| | DCWVRDSRYQKAFQEIPENDPDGWECKEGYLH | |
| | VVGPSKVEFSDKKGDVINNFQGTLPSVPNDWKT | |
| | IRTNDFKNRKRKNEPVFCCEDDKGNYYTMAKY | |
| | CETFFFDLKENEEYEIPEKARIKYKELLRVYNNN | |
| | PQAVPESVFQSRVARENVEKLKSGDLVYFKHNE | |
| | KYVEDIVPVRISRTVDDRMIGKRMSADLRPCHG | |
| | DWVEDGDLSALNAYPEKRLLLRHPKGLCPACR | |
| | LFGTGSYKGRVRFGFASLENDPEWLIPGKNPGD | |
| | PFHGGPVMLSLLERPRPTWSIPGSDNKFKVPGR | |
| | KFYVHHHAWKTIKDGNHPTTGKAIEQSPNNRT | |
| | VEALAGGNSFSFEIAFENLKEWELGLLIHSLQLE | |
| | KGLAHKLGMAKSMGFGSVEIDVESVRLRKDW | |
| | KQWRNGNSEIPNWLGKGFAKLKEWFRDELDFI | |
| | ENLKKLLWFPEGDQAPRVCYPMLRKKDDPNGN | |
| | SGYEELKDGEFKKEDRQKKLTTPWTPWA* | |
| DpbaCas7-11 | ASEDDDTPTLRKVLKDEINGQEDMWRKFCEAL | 1367 |
| (SEQ ID NO: 7) | GNSLYDLSKKAKERKRTEALPRLLGETEIYGLP | |
| [RLC15988.1] | MRENKEDEPLPSSLTYKFKWLIAGELRAETPFFF | |
| | GTEVQEGQTSATILLNRDGYFRLPRSVIRGALRR | |
| | DLRLVMGNDGCNMPIGGQMCECGVCRVMRHI | |
| | VIEDGLSDCKIPPEVRHRIRLNCHTGTVEEGALF | |
| | DMETGYQGMTFPFRLYCETENSDLDSYLWEVL | |
| | NNWQNGQSLFGGDTGTGFGRFELTEPKVFLWN | |
| | FSKKEKHEAYLLNRGFKGQMPVQDVKTKSFKT | |
| | KTWFQIHRELDISPKKLPWYSTDYRFNVTSPLIS | |
| | RDPIGAMLDPRNTDAIMVRKTVFCPDPNAKNRP | |
| | APATVYMIKGESIRGILRSIVVRNEELYDTDHED | |
| | CDCILCRLFGSIHQQGSLRFEDAEVQNSVSDKK | |
| | MDHVAIDRFTGGGVDQMKFDDYPLPGCPAQPL | |
| | ILEGKFWVKDDIDDESKSALEKAFADFRDGLVS | |
| | LGGLGAIGYGQIGDFELIGGSADWLNLPKPEEN | |
| | RTDVPCGDRSAQGPEIKISLDADKIYHPHFFLKP | |
| | SDKNVYRERELVSHAKKKGPDGKSLFTGKITCR | |
| | LSTEGPVFIPDTDLGEDYFEMQASHKKHKNYGF | |
| | FRINGNVAIPGSSIRGMISSVFEALTNSCFRVFDQ | |
| | ERYLSRSEKPDPTELTKYYPGKVKRDGNKFFIL | |
| | KMKDFFRLPLYDFDFEGEAESLRPNYDEDRNEE | |
| | ENKGKNKNTQKVKNAVEFNIKMAGFAKHNRD | |
| | FLKKYKEQEIKDIFMGKKKVYFTAGKHKPNEA | |
| | HDNDKIALLTKGSNKKAEKGYFKFTGPGMVNV | |
| | KAGVEGEECDFHIDESDPDVYWNMSSILPHNQI | |
| | KWRPSQKKEYPRPVLKCVKDGTEYVMLKRSEH | |
| | VFAEASSEDSYPVPGKVRKQFNSISRDNVQNTD | |
| | HLSSMFQSRRLHDELSHGDLVYFRHDEKRKVT | |
| | DIAYVRVSRTVDDRPMGKRFKNESLRPCNHVC | |
| | VEGCDECPDRCKELEDYFSPHPEGLCPACHLFG | |
| | TTDYKGRVSFGLGWHESNTPKWYMPEDNSQK | |
| | GSHLTLPLLERPRPTWSMPNKKSEIPGRKFYVH | |
| | HPWSVDKIRNRQFDPAKEKQPDDVIKPNENNRT | |
| | VEPLGKGNEFTFEVRFNNLREWELGLLLYSLEL | |
| | EDNMAHKLGMGKALGMGSARIKAEAIELRCES | |
| | AGQNAELKDKAAFVRKGFEFLEIDKPGENDPM | |
| | NFDHIRQLRELLWFLPENVSANVRYPMLEKEDD | |
| | GTPGYTDFIKQEEPSTGKRNPSYLSSEKRRNILQ | |
| | TPWKHWYLIPPFQASAQSETVFEGTVKWFDDK | |
| | KGFGFIKINDGGKDVFVHHSSIVGTGFKSLNEGD | |
| | SVAFKMGVGPKGPCAEKVKKIGN | |
| DsbaCas7-11 | MKITLRFLEPFRMLDWIRPEERISGNKAFQRGLT | 1659 |
| (SEQ ID NO: 8) | FARWHKSKADDKGKPFITGTLLRSAVIRAAEHL | |
| [OQY58162.1] | LVLSKGKVGEKACCPGKFLTETDTETNKAPTMF | |
| | LRKRPTLKWTDRKGCDPDFPCPLCELLGPGAVG | |
| | KKEGEAGINSYVNFGNLSFPGDTGYSNAREIAV | |
| | RRVVNRVDYASGKAHDFFRIFEVDHIAFPCFHG | |
| | EIAFGENVSSQARNLLQDSLRFTDRLCGALCVIR | |
| | YDGDIPKCGKTAPLPETESIQNAAEETARAIVRV | |
| | FHGGRKDPEQAQIDKAEQIQLLSAAVRELGRDK | |
| | KKVSALPLNHEGKEDHYLWDKKAGGETIRTILK | |
| | AAAEKEAVANQWRQFCIELSEELYKEAKKAHG | |
| | GLEPARRIMGDAEFSDKSVPDTVSHSIGISVEKE | |
| | TIIMGTLKAETPFFFGIESKEKKQTDLMLLLDGQ | |
| | NHYRIPRSALRGILRRDIRSVLGTGCNAEVGGRP | |
| | CLCPVCRIMKNITVMDTRSSTDTLPEVRPRIRLN | |
| | PFTGSVQEKALFNMEMGTEGIEFPFVLSYRGKK | |
| | TLPKELRNVLNWWTEGKAFLGGAASTGKSIFQL | |
| | SDIHAFSSDLSDETARESYLSNHGWRGIMENSIV | |
| | HESPLEGGAGGCSFGLSDLPKLGWHAEDLKLSD | |
| | IEKYKPFHRQKISVKITLNSPFLNGDPVRALTED | |
| | VADIVSFKKYTQGGEKIIYAYKSESFRGVVRTAL | |
| | GLRNQGNDDITGKKNVPLIALTHQDCECMLCRF | |
| | FGSEYEAGRLYFEDLTFESEPEPRRFDHVAIDRF | |
| | TGGAVNQKKFDDRSLVPGKEGFMTLIGCFWMR | |
| | KDKELSRNEIEELGKAFADIRDGLYPLGAKGSM | |
| | GYGQVAELSIVDDEDSDDENNPAKLLAESMKN | |
| | ASPSLGTPTSLKKKDAGLSLRFDENADYYPYYF | |
| | LEPEKSVHRDPVPPGHEEAFRGGLLTGRITCRLT | |
| | VRTPLIVPNTETDDAFNMKEKAGKKKDAYHKS | |
| | YRFFTLNRVPMIPGSEIRGMISSVFEALSNSCFRI | |
| | FDEKYRLSWRMDADVKELEQFKPGRVADDGK | |
| | RIEEMKEIRYPFYDRTYPERNAQNGYFRWDARI | |
| | SLTDNSMRKMEKDGVPRNVIYKLNTLKNKAYK | |
| | SEKSFLFDLKNKAGGVGRYKKLVLKHAEVRGG | |
| | EIPYYSHPTPTDCKLLSLVGPNRQLCRQDTLVQ | |
| | YRIIKHRRGAKPEEDFMFVGTPSENQKGHKENN | |
| | DHGGGYLKISGPNKIEKENVLTSGVPSVPENMG | |
| | AVVHNCPPRLVEVTVRCGRKQEEECKRKRLVP | |
| | EYVCADPEKKVTYTMTKRCERIFLEKSRRIIPFT | |
| | NDAVDKFEILVKEYRRNAEQQDTPEAFQTILPE | |
| | NGTVNPGDLLYFREEKGKAAEIVPVRISRKVDD | |
| | RHIGKRIDPELRPCHGEWIEDGDLSKLDAYPAE | |
| | KKLLTRHPKGLCPACRVFGTGSYKSRVRFGFAA | |
| | LKGTPKWLKEDPAEPSQGKGITLPLLERPRPTW | |
| | AVLHNDKENSEIPGRKFYVHHNGWKGISEGIHPI | |
| | SGENIEPDENNRTVEVLDKGNRFVFELSFENLEP | |
| | RELGLLIHSLQLEKGLAHKLGMAKSMGFGSVEI | |
| | DVESVRVKHRSGEWDYKDGETVDGWIEEGKR | |
| | GVAAKGKANDLRKLLYLPGEKQNPHVHYPTLK | |
| | KEKKGDPPGYEDLKKSFREKKLNRRKMLTTLW | |
| | EPWHK | |
| FmCas7-11 | MPRFQLSLTFFDEPFRLIEWTDKSNRNSANTQW | 1610 |
| (SEQ ID NO: 9) | MRGQGFARWHKITLEKGFPFVTGTAVRSKIIRE | |
| | VEALLSRNKGTWNGIPCCSGFFDTKGPSPTHLR | |
| | YRPTLEWEYGKTVCTSEADVCPLCLLLGRFDQA | |
| | GKKSDTPCQSTDYHVHWENLSAGVAQYRLEDI | |
| | AQKRTSNRVDFFSKKAHDHYGVWEVTAVKNL | |
| | LGYIYISDAITESHQKTVISLLKAALSFTDTLCGA | |
| | NCKLELSDEPVDSIHSNQSASNFNPHSGAAPSQC | |
| | SQSMPPFNMDQETKELANTLCKAFTGNMRHLR | |
| | TLADAVREMRRMSPGISSLPRGRLNKEGEITAH | |
| | YLWDERIDEKTIRQVLEDTIELSPARSIIYKNWIS | |
| | FCNQLGQKLYERAKDNDPILERKRPLGEAAFSK | |
| | VPTSSHAPRHDMNSRVKGGFTREWIIVGTLRAL | |
| | TPFYMGTGSQAGKQTSMPTLQDSNDHFRLPRT | |
| | ALRGALRRDINQASDGMGCVVELGPHNLCSCP | |
| | VCQVLRQIRLLDTKSKFSMPPAIRQKICKNPVLSI | |
| | VNEGSLFDVELGIEGETFPFVMRYRGGAKIPDTII | |
| | TVLSWWKNERLFIGGESGTGRGRFVLECPRIFC | |
| | WDVEKGQNDYIQYHGFRNKEDELLSVYSTVSG | |
| | LAEKNDVNLNNARDFSFDKICWEVQFDGPVLT | |
| | GDPLAALFHGNTDSVFYKKPILKSGEKEPSYQW | |
| | AIKSDTVRGLIRSAFGKRDALLIKSHEDCDCLLC | |
| | EAFGSKHHEGKLRFEDLTPKSDEIKTYRMDHVA | |
| | IDRISGGAVDQCKYDDEPLVGTSKHPLVFKGMF | |
| | WINRDSSVEMQRALIAAFKEIRDGLYPLGSNGG | |
| | TGYGWISHLAITNGPDWLNLEEVPLPQPTADIPV | |
| | EECTAEPYPKFQKPDLDQNAVYYPHYFLQPGKP | |
| | AERERHPVSHDHIDDKLLTGRLVCTLTTKTPLIIP | |
| | DTQTNTMLPPNDAPEGHKSFRFFRIDDEVLIPGS | |
| | EIRGMVSTVFEALTGSCFRVINQKAHLSWRINA | |
| | DMAKHYRPGRIIQNNEKMFIQPYKMFRLPFYAG | |
| | FDPRNCLSEKQLLGIEPVKLWVKDFVASLVKPQ | |
| | TDIDIEWKEKIGFVRVTGPNKVEVDSSNTPDPSL | |
| | PECESDWKDIHITEDGSTPSKNDRVYRCQLKGV | |
| | TYTVAKWCEAFWVKDEGKKPITVNAEAINRYH | |
| | LIMKSYQDNPQSPPIIFRSLPVLNYKQDQKIIGSM | |
| | IFYRESAKSDKIVNEIIPVKISRTADTELLAKHLP | |
| | NNDFLPCAATCLNECDTCNAKTCKFLPLYREGY | |
| | PVNGLCPSCHLFGTTGYQGRVRFGFAKMNGNA | |
| | KFCQGGERPEDRAVTLPLQERPKLTWVMPNEN | |
| | STIPGRKFFLHHQGWKKIVDEGKNPINGDVIEPD | |
| | ANNRTVEPLAAGNDFSFEVFFENLREWELGLLR | |
| | YTLELESELAHKLGMGKAFGFGSVKIKIKSVDL | |
| | RKQGEWEKATNTLVSEDKKSSWYNIHTVNNLR | |
| | TALYYVEDDKIQVNYPKLKKDNESDNRPGYVE | |
| | MKKTAFPVRDILTTPWWPWWPPTPPPMNQSGN | |
| | QSYARSEEPARITESQPEVYKTGTVKFYKHDKK | |
| | FGFITMDGRENIHFAGNQICRPETSLQSGDKVKF | |
| | IEGENYKGPTALKVERLKG | |
| GwCas7-11 | MTKKPGTEDKATLWGKESASKSVKTILEESIQG | 1400 |
| (SEQ ID NO: 10) | FTVEQKRSFFANLADQLVSRAGEQGAKSVRSQ | |
| | GLIIGRKENYAKPSAQEPTRHHLYRQPSNASAFL | |
| | ATGWLIAETPFFIGSGTEGQKQTDDQAESLHLRT | |
| | LRDGHGRFRIPFTTIRGVMDKELRDILQAGCAK | |
| | GRSLRAPCPCQVCTLMRRIQVRDAIAADILPPDL | |
| | RMRTRIDPSHGTVAHLFSLEMAPQGLKLPFFLK | |
| | LKGVETIDPDKELLEILNDWSAGQCFLGGLWGT | |
| | GKGRFRLDDLQWHRLELDNADYYTPLLQDRFF | |
| | AGETISDLRQGLQSINIQPERIPAQTPSRNMPYCR | |
| | VDCILEFKSPVLSGDPVAALFESDAPDNVAYKK | |
| | PVVQYDETGRLRTTDPGPVEMLTCLKGEGVRG | |
| | VVAYLAGKAYDQHDLSHDSCNCTFCQAFGNG | |
| | QKAGSLRFDDFMPVQFESDQAGNFSWSPHTPH | |
| | AMRSDRVALDVFGGAMPEAKFDDRPLAASPGK | |
| | PLNFKSTIWYREDMGKEAGKALKRALIDLQNN | |
| | MAAIGSGGGIGRGWVSRVCFEGDIPDFLEDFPEP | |
| | ITVTEPEQDSQLLKNQAVADETAVSACDTADAP | |
| | HPLAVTLEPGARYFPRVIIPRAPTVKRDECVTGQ | |
| | RYHTGRLSGKIFCELNTLGPLFVPDTDYSAGVP | |
| | VPISDEQLAECQLQAVFENTSKFNEFFATYPEET | |
| | VTKLKDLLCAADDKWILAVKDITADLRQEIGED | |
| | TFQRIIRKAGHKTQRFHQINDEIGLPGASLRGMV | |
| | LSNYQILTNSCYRNLKATEEITRRMPADEAKYR | |
| | KAGRVTVSGDGAQKKYSIQEMEVLRLPIYDNM | |
| | NTPDNMPDVAKQATTAKRCNNLMNEAAKTSR | |
| | VELKARWREGQSKIKYQIIDALNKVDPIIQVISSS | |
| | KQINPNNGKTGWGYVKYTGANVFAKSLVAPID | |
| | CLRKKDAGHVCCQVNLNPAWEASNFDILINEK | |
| | CPVERQSGPRPTLRCKGQDSAWYTLTKRSERIF | |
| | TDKKPVPDPINIPPREVKRYNELRDSYKKNTAH | |
| | VPKPLQTFFNQESLANGDLVYFEVNQFGEASQL | |
| | TPVSISRTTDLFPIGGRLPQGHKDLFPCTAMCLS | |
| | ECKNCVPASFCEFHSRSHEKLCPACSLAGTTGN | |
| | RGRIKFSEAWLSGLPKWHSVSQDNVGRGLGVT | |
| | MPRLERSRRTWHLPTKDAYLLGQSIYLNHPVPA | |
| | ILPSDQVPSENNQTVEPLGPKNIFSFQLAFDNLSI | |
| | EELGLLLYSLELESGMAHRLGRGRALGMGSVQI | |
| | SVKDIQIRDNKSFLFSSNISKKSEWIQCGKDEFA | |
| | QEAWFGESWDNIDHIQRLRQALTIPVKGDVGCI | |
| | RYPKLEAEGGMPDYIKLRKRLTPLCDREEPVRY | |
| | RINPVQLARMILPFVPWHGACPALLNEQVMIEA | |
| | KRLTELDRANWPC | |
| HreCas7-11 | MSVEEFYVRLTFLEPFRVVPWVRNGDERKGDRI | 1720 |
| (SEQ ID NO: 11) | YQRGGTYARWHKINDSHGQPYITGTMLRSAVL | |
| | REIENTLTLHNTYGCCPGGTRTTEGKLEKPLYLR | |
| | RRDGFEFENHAEKPCSEEDPCPLCLIQGRFDKLR | |
| | RDEKKQFVRQGNISFCSVNFSNLNISSGIKSFSW | |
| | EEIAVSRVVNRVDPNSGKAKDFFRVWEIDHKLC | |
| | PNFLGKMSISLSEKLEDVKALLAVGLAQVNVLS | |
| | GALCRVDIIDPETQKDTVHQHLIQQFVTRIQDKE | |
| | KGDAADIPAFTLPPAGLSPSSNEWNDTIKSLAEK | |
| | IRKIKELEQGQKLRQMADVIRELRRKTPAYLDQ | |
| | LPAGKPEGRESIWEKTPTGETLTLRQLLKSANVP | |
| | GESWRAFCEELGEQLYRLEKNLYSHARPLPRLL | |
| | GETEFYGQPARKSDDPPMIRASYRAFPSYVWVL | |
| | DGILRAETPFYFGTETSEGQTSQAIILCPDGSYRL | |
| | PRSLLRGVIRRDLRAILGTGCNVSLGKVRPCSCP | |
| | VCEIMRRITVQQGVSSYREPAEVRQRIRSNPHTG | |
| | TVEEGALFDLETGPQGMTFPFRLYFRTRSPYIDR | |
| | ALWLTINHWQEGKAIFGGDIGVGMGRFRLENL | |
| | QIRSADLVSRRDFSLYLRARGLKGLSREEVTRIG | |
| | LNEEQWEAVMADDPGTHYNPFPWEKISYTLLIH | |
| | SPLISNDPIAAMLDHDNKDAVMVQKTVLFVDES | |
| | GNYSQMPHHFLKGSGIRGACRFLLGRKDAPNE | |
| | NGLTYFEADHEECDCLLCSLFGSKHYQGKLRFE | |
| | DAELQDEVEAIKCDHVAIDRFHGGTVHRMKYD | |
| | DYPLPGSPNRPLRIKGNIWVKRDLSDTEKEAVK | |
| | DVLTELRDGLIPLGANGGAGYGRIQRLMIDDGP | |
| | GWLALPERKEDERPQPSFSPVSLGPVHVNLKSG | |
| | SDTADVYYYHPHYFLEPPSQTVSRELDIISHART | |
| | RDSGGEALLTGRILCRLITRGPIFIPDTNNDNAFG | |
| | LEGGIGHKNYRFFRINDELAIPGSELRGMVSSVY | |
| | EALTNSCFRIMEEGRYLSRRMGADEFKDFHPGI | |
| | VVDGAKIREMKRYRLPLYDTPDKTSRTKEMTC | |
| | PELFTRKDGRPERAKKFNEEIAKVAVQNRAYLL | |
| | SLDEKERREVLLGNREVTFDECPDDEYSDDEYS | |
| | ELKYAQKYKDFIAVLKKNGQKRGYIKFTGPNT | |
| | ANKKNEDAPDKNYRSDWDPFKLNILLESDPECR | |
| | VSNIHCYPRPLLVCIKDKAEYRIHKRCEAIFCSIG | |
| | SPSDLYDIPQKVSNQYRTILQDYNDNTGKIVEIF | |
| | RTQIKHDQLTTGDLVYFKPAANGQVNAVIPVSI | |
| | SRKTDENPLAKRFKNDSLRPCAGLCVEDCNECP | |
| | ARCKKVADYFNPHPRGLCPACHLFGTTFYKGR | |
| | VRFGFAWLTGEDGAPRWYKGPDPCDSGKGRP | |
| | MTIPLLERPRPTWSIPDNSFDIPGRKFYVHHPYS | |
| | VDGIDGETRTPNNRTIEPLAEGNEFVFDIDFENL | |
| | RDWELGLLLYSLELEDSLAHKLGLGKPLGFGTV | |
| | QINIRGISLKNGSKGWDTKTGDDKNQWIKKGFA | |
| | HLGIDIKEANERPYIKQLRELLWVPTGDNLPHV | |
| | RYPELESKTKDVPGYTSLLKEKDLADRVSLLKA | |
| | PWKPWKPWSGTAPHPDKGTNRLRASIVERDRIQ | |
| | RKTDTAKPEKKEETKVGKSSSSDIEKRYVGTVK | |
| | WFNDKKGYGFILYGTDEEIFVHRSGVADNSIPK | |
| | EGQKVGFRIERGARGSHAVEVKAIE | |
| HsmCas7-11 | MTKIPISLTFLEPFRLVDWVSESERDKSEFLRGLS | 1548 |
| (SEQ ID NO: 12) | FARWHRIKNQREDENQGRPYITGTLLRSAVIKA | |
| | AEELIFLNGGKWQSEECCNGQFKGSKAKYRKV | |
| | ECPRRRHRATLKWTDNTCSDYHNACPFCLLLG | |
| | CLKPNSKENSDIHFSNLSLPNKQIFKNPPEIGIRRI | |
| | LNRVDFTTGKAQDYFYVWEVEHSMCPKFQGTV | |
| | KINEDMPKYNVVKDLLISSIQFVDKLCGALCVIE | |
| | IGKTKNYICQSFSSNIPEEEIKKLAQEIRDILKGED | |
| | ALDKMRVLADTVLQMRTKGPEIVNELPRGIEKK | |
| | GGHWLWDKLRLRKKFKEIANNYKDSWQELCE | |
| | KLGNELYISYKELTGGIAVKKRIIGETEYRKIPEQ | |
| | EISFLPSKAGYSYEWIILGKLISENPFFFGKETKTE | |
| | EQIDMQILLTKDGRYRLPRSVLRGALRRDLRLVI | |
| | GSGCDVELGSKRPCPCPVCRIMRRVTLKDARSD | |
| | YCKPPEVRKRIRINPLTGTVQKGALFTMEVAPE | |
| | GISFPFQLRFRGEDKFHDALQNVLVWWKEGKL | |
| | FLGGGASTGKGRFKLEIEHVLKWDLKNNFHSYL | |
| | QYKGLRDKGDFNSIKEIEGLKVETEEFKVKKPFP | |
| | WSCVEYTIFIESPFVSGDPVEAVLDSSNTDLVTF | |
| | KKYKLEESKEVFAIKGESIRGVFRTAVGKNEGK | |
| | LTTENEHEDCTCILCRLFGNEHETGKVRFEDLEL | |
| | INDSAPKRLDHVAIDRFTGGAKEQAKFDDSPLIG | |
| | SPDSPLEFTGIVWVRDDIDEEEKKALKSAFLDIK | |
| | SGYYPLGGKKGVGYGWVSNLKIESGPEWLRLE | |
| | VQEKSSQENVLSPVILSEVMDIEFNPPKIDENGV | |
| | YFPYAFLRPLNEVKRTREPIGHNEWKKSLISGYL | |
| | TCRLELLTPLIIPDTSEEVIKEKVNNGEHPVYKFF | |
| | RLGGHLCIPAAEIRGMISSVYEALTNSCFRVFDE | |
| | KRLISWRMTAEEAKRPDPKKSEEQNRMRFRPG | |
| | RIIKKDKKFYAQEMLELRIPVYDNKDKRNEISQ | |
| | NDPTRPSEYNHPTEPERIFFSNAEKIRNFLKRNSN | |
| | YLHGSTPLLFRQWSISNRYDKIALIGNKSQGHLK | |
| | FTGPNKIEVSEGTKCPKYETIPGRDEWDKAVHN | |
| | YVEPGKFVTVISRKKGQKPKAVQRRRNVPAFCC | |
| | YDYNTNRCFVMNKRCERVFKVSRDKPKYEIPP | |
| | DAIRRYEHVLRKYRENWERYDIPEVFRTRLPGD | |
| | GETLNEGDLVYFRLDENNRVLDIIPVSISRISDTQ | |
| | YLGRRLPDHLRSCVRECLYEGWGDCKPCKLSLF | |
| | PEKMWIRINPEGLCPACHLFGTQVYKGRVRFGF | |
| | ARAGSNWKFREEQLTLPRFETPRPTWVIPKRKD | |
| | EYQIPGRKFYLHHNGWEEIYKKNKKNEIKKEKN | |
| | NATFEVLKQGTFYFKVFFENLELWELGLLIFSAE | |
| | LGGEEFAHKLGHGKALGFGSVKISVDKIILRRDP | |
| | GQFEQRGQKFKRDAVDKGFCVLENRFGKTNFK | |
| | IYLNNFLQLLYWPNNKKVKVRYPYLRQEDDPE | |
| | KLPGYVELKKHQMLKDDNRYSLFARPRAVWL | |
| | KWTEMVQRDKS | |
| HvmCas7-11 | MTQITIQVTFFHPFRVVPWNHRDHRKTDRKYLR | 1723 |
| (SEQ ID NO: 13) | GGTFAKWHCTASEGKSGRPYITGTLLRSALFAEI | |
| | EKLIAFHDPFKCCRGKDKTENGNAKPLFLRRRP | |
| | RADCDPCGTCPLCLLMGRSDTVRRDAKKQKKD | |
| | WSVHFCNLREATERSFNWKETAIERIVNRVDPS | |
| | SGKAKDYMRIWEIDPLVCSQFNGIITINLDTDNA | |
| | GKVKLLMAAGLAQINILAGSICRADIISEDHDAL | |
| | IKQFMAIDVREPEVSTSFPLQDDELNNAPAGCG | |
| | DDEISTDQPVGHNLVDRVRISKIAESIEDVESQE | |
| | QKAQQLRRMADAIRDLRRSKPDETTLDALPKG | |
| | KTDKDNSVWDKPLKKDILPSPRMPASEDDDTPT | |
| | LRKVLKDEINGQEDMWRKFCEALGNSLYDLSK | |
| | KAKERKRTEALPRLLGETEIYGLPMRENKEDEP | |
| | LPSSLTYKFKWLIAGELRAETPFFFGTEVQEGQT | |
| | SATILLNRDGYFRLPRSVIRGALRRDLRLVMGN | |
| | DGCNMPIGGQMCECGVCRVMRHIVIEDGLSDC | |
| | KIPPEVRHRIRLNCHTGTVEEGALFDMETGYQG | |
| | MTFPFRLYCETENSDLDSYLWEVLNNWQNGQS | |
| | LFGGDTGTGFGRFELTEPKVFLWNFSKKEKHEA | |
| | YLLNRGFKGQMPVQDVKTKSFKTKTWFQIHRE | |
| | LDISPKKLPWYSTDYRFNVTSPLISRDPIGAMLD | |
| | PRNTDAIMVRKTVFCPDPNAKNRPAPATVYMIK | |
| | GESIRGILRSIVVRNEELYDTDHEDCDCILCRLFG | |
| | SIHQQGSLRFEDAEVQNSVSDKKMDHVAIDRFT | |
| | GGGVDQMKFDDYPLPGCPAQPLILEGKFWVKD | |
| | DIDDESKSALEKAFADFRDGLVSLGGLGAIGYG | |
| | QIGDFELIGGSADWLNLPKPEENRTDVPCGDRS | |
| | AQGPEIKISLDADKIYHPHFFLKPSDKNVYRERE | |
| | LVSHAKKKGPDGKSLFTGKITCRLSTEGPVFIPD | |
| | TDLGEDYFEMQASHKKHKNYGFFRINGNVAIPG | |
| | SSIRGMISSVFEALTNSCFRVFDQERYLSRSEKPD | |
| | PTELTKYYPGKVKRDGNKFFILKMKDFFRLPLY | |
| | DFDFEGEAESLRPNYDEDRNEEENKGKNKNTQ | |
| | KVKNAVEFNIKMAGFAKHNRDFLKKYKEQEIK | |
| | DIFMGKKKVYFTAGKHKPNEAHDNDKIALLTK | |
| | GSNKKAEKGYFKFTGPGMVNVKAGVEGEECDF | |
| | HIDESDPDVYWNMSSILPHNQIKWRPSQKKEYP | |
| | RPVLKCVKDGTEYVMLKRSEHVFAEASSEDSYP | |
| | VPGKVRKQFNSISRDNVQNTDHLSSMFQSRRLH | |
| | DELSHGDLVYFRHDEKRKVTDIAYVRVSRTVD | |
| | DRPMGKRFKNESLRPCNHVCVEGCDECPDRCK | |
| | ELEDYFSPHPEGLCPACHLFGTTDYKGRVSFGL | |
| | GWHESNTPKWYMPEDNSQKGSHLTLPLLERPR | |
| | PTWSMPNKKSEIPGRKFYVHHPWSVDKIRNRQF | |
| | DPAKEKQPDDVIKPNENNRTVEPLGKGNEFTFE | |
| | VRFNNLREWELGLLLYSLELEDNMAHKLGMGK | |
| | ALGMGSARIKAEAIELRCESAGQNAELKDKAAF | |
| | VRKGFEFLEIDKPGENDPMNFDHIRQLRELLWF | |
| | LPENVSANVRYPMLEKEDDGTPGYTDFIKQEEP | |
| | STGKRNPSYLSSEKRRNILQTPWKHWYLIPPFQA | |
| | SAQSETVFEGTVKWFDDKKGFGFIKINDGGKDV | |
| | FVHHSSIVGTGFKSLNEGDSVAFKMGVGPKGPC | |
| | AEKVKKIGN | |
| HvsCas7-11 | MKSIPITLTFLEPYRILPWAEKGKRDKKEYLRGA | 1528 |
| (SEQ ID NO: 14) | NYVRLHKDKNGKFKPYITGTLIRSAVLSAIEMLL | |
| | DITNGEWNGKECCLAKFHTEGEKPSFLRKKPIYI | |
| | RAEKDEICTSRETACPLCLILGREDKAEKKEKDK | |
| | EKFDVHFSNLNLYSSKEFSTIEELAPKRALNRIE | |
| | QYTGKAQDYFTVYEALNKEFWTFKGRIRIKEDI | |
| | YDKVTDLLFSALRCVEKIAGALCRIEIDKEPSQQ | |
| | KGFVKRQLSKQAKEDIEKIFQVVKDAQKLRLLS | |
| | DCFRELTRMANKDELALPLGPEDDGHYLWDKI | |
| | KVEGKTLRIFLRNCFSQYKDNWLCFCDEASKKG | |
| | YQKYREKRHKLTDRELPTATPKHFAEKKDPQIS | |
| | PIYIDKDDKVYEWIIVGRLIAQTPFHFGDEEKAE | |
| | GAILLTPDNRFRLPRTALRGILRRDLKLAGASAC | |
| | EVEVGRSEPCPCDVCKIMRRVTLLDTVSEDLRD | |
| | FLPELRKRIRINPQSGTVAEGALFDTEVGPEGLSF | |
| | PFVLRYKCEKLPDSLTTVLCWWQEGLAFLSGES | |
| | ATGKGRFRLEINGAFVWDLQKGLFNYIKNHGFR | |
| | GEERLFLEGNEAELEKMGIQINTELLQPEMIKKE | |
| | KNFTDFPYDLIKYQLNISSPLLLNDPIRAIALYEG | |
| | EGKAPDAVFFKKYVFENGKIEEKPCFKAESIRGI | |
| | FRTAVGRIKNVLTKNHEDCICVLCHLFGNVHET | |
| | GRLKFEDLKIVSGQEEKFFDHVAIDRFLGGAKE | |
| | KYKFDDKPIIGAPDTPIVLEGKIWVKKDINDEAK | |
| | ETLSQAFSDINTGIYYLGANGSIGYGWIEEVKAL | |
| | KAPSWLKIKEKPNFEKDTSLNISAIMNEFKKDIQ | |
| | TLNLDKTYLPYGFLKLLEKVKRTSSPITHERFYE | |
| | NHLTGFIECSLKVLSPLIIPDTETPEKEENGHKYY | |
| | HFLKIDNKPIIPGAEIRGAVSSIYEALTNSCFRVF | |
| | GEKKVLSWRMEGKDAKEFMPGRVSKKKGKLY | |
| | MVKMQALRLPVYDNPALANEIRSGSIYEKYKNS | |
| | KVEIIFFQTVEGIRKFLRGNFNNVEWKKVLVTGI | |
| | DPLAILPSQKIPGNDKWVKNLQSKISPVRGYFKF | |
| | TGPNKIETKRREEEKDEKLRTKANKVSCLQKDK | |
| | WYEAMHNHVEYKQDYTPPNSPKTEPLERPRNIP | |
| | CFVCSDKEKIYRMTKRCERVFVSLGENAPKYEI | |
| | PISAIKRYEVILSAYRENWERNKTPELFRTRLPG | |
| | DGRTLNEDDLVYFRADENEKVKDIIPVCISRIVD | |
| | EVPLIKRLSQELWPCVLAECPLLGFECKKCELEG | |
| | LPEKIWFRINKDGLCPACRLFGTQIYKSRVRFSF | |
| | AYAKNWKFYDGYITLPRLESPRATWLILKEKDK | |
| | HYIKYKVCGRKFYLHNSTYEDIINNSKKEKEKK | |
| | TENNASFEVLKEGEFTFKVYFENLENWELGLLL | |
| | LSLTGLGEAIKIGHAKPLGFGSVKIEAKKIYFREE | |
| | AGKFHPCEKADEYLKKGLNKLTSWFGKNEINE | |
| | HMRNLLLFMTYYQNLPKVKYPDFDGYAKWRC | |
| | SYVEQDKVEYFQNRWIVAS* | |
| OmCas7-11 | MIPDLRSLVVHISFLTPYRQAPWFPPEKRRNNNR | 1801 |
| (SEQ ID NO: 15) | DWLRMQSYARWHKVAPEEGHPFITGTLLRSRVI | |
| | RAVEEELCLANGIWRGVACCPGEFNSQAKKKP | |
| | KHLRRRTTLQWYPEGAKSCSKQDGRENACPFC | |
| | LLLDRFGGEKSEEGRKKNNDYDVHFSNLNPFYP | |
| | GSSPKVWSGPEEIGRLRTLNRIDRLTTKAQDFFR | |
| | IYEVDQVRDFFGTITLAGDLPRKVDVEFLLRRGL | |
| | GFVSTLCGAQCEIKVVDLKKKQNNKEDSILPVS | |
| | EVPFFLEPEVLAKMCQDVFPSGKLRMLADVILR | |
| | LREEGPDNLTLPMGSQGLGGRLPHHLWDVPLV | |
| | SKDRETQTLRSCLEKIAAQCKSEQTQFRLFCQKL | |
| | GSSLFRINKGVYLAPNSKISPEPCLDPSKTIRTKG | |
| | PVPGKQKHRFSLLPPFEWIITGTLKAQTPFFIPDE | |
| | QGSHDHTSRKILLTRDFYYRLPRSLLRGIIRRDL | |
| | HEATDKGGCRVELAPDVPCTCQVCRLLGRMLL | |
| | ADTTSTTKVAPDMRHRVGVDRSCGIVRDGALF | |
| | DTEYGIEGVCFPLEIRYRGNKDLEGPIRQLLSW | |
| | WQQGLLFLGGDFGIGKGRFRLENMKIHRWDLR | |
| | DESARADYVQKCGLRRGVGDDTAINLEKDLSL | |
| | NLPESGYPWKKHAWKLSFQVPLLTADPIMAQT | |
| | RHEEDSVYFQKRIFTSDGRVVLVPALRGEGLRG | |
| | LLRTAVSRAYGISLINDEHEDCDCPLCKIFGNEH | |
| | HAGMLRFDDMVPVGTWNDKKIDHVSCSRFDAS | |
| | VVNKFDDRSLVGSPDSPLHFEGTFWLHRDFQND | |
| | VEIKTALQDFADGLYSIGGKGGIGYGWLFDMEI | |
| | PRSLRKLNSGFREASSIQDALLDSAKEIPLSAPLT | |
| | FTPVKGAVYNPYYYLPFPAEKPERCLVPPSHAR | |
| | LQSDRYTGCLTCELETVSPLLLPDTCREKDGNY | |
| | KEYPSFRLNNTPMIPGAGLRAAVSQVYEVLINS | |
| | CIRIMDQGQTLSWRMSTSEHKDYQPGKITDNGR | |
| | KIQPMGKQAIRLPLYDEVIHHVSTPGDTDDLEK | |
| | LKAIVLELTRPWKELPEEQKKKRFEKCKNILDG | |
| | RMLQQKELRALENSGFAYWRDKTSLTFDSFLK | |
| | DAIEQEYPRYSGDYQRIKALVVNITLPWKLLKK | |
| | EERHKRFDKCRRILKGQQPLTKDERKALEESGF | |
| | ANWHGRELLFDRFLKDENSCLIKAETTDRVIAS | |
| | VAKNNRDYLFEIKQQDFARYKRIIQGLERVPFSL | |
| | RSLAKSKETSFQIACLGLRRGRFLRKGYLKISGP | |
| | NNANVEISGGSHSNSGYSDIWDDPLDFSFRLSG | |
| | KSELRPNTQKTREYPRPSFTCTVDGKQYTVNKR | |
| | CERVFEDSAAPAIELPRMVREGYKGILTDYEQN | |
| | AKHIPQGFQTRFSSYRELNDGDLVYYKTDSQGR | |
| | VTDLAPVCLSRLADDRPLGKRLPEEYRPCAHVC | |
| | LEECDPCTGKDCPVPIYREGYPARGFCPACQLF | |
| | GTQMYKGRVRFSFGVPVNSTRSPQLKYVTLPSQ | |
| | ERPRPTWVLPESCKGKEKDVPGRKFYLRHDGW | |
| | REMWGDDDKPDSRPSSEECQDIIEGIGPGEKFHF | |
| | RVAFENLDKNELGRLLYSLELDAGMNHHLGRG | |
| | KAFGFGQVKIRVTKLERRLEPGQWRSEKICTDL | |
| | PVTSSELVISSLKKVEERRKLLRLVMTPYKGLTA | |
| | CYPGLERENGRPGYTDLKMLATYDPYRELVVQI | |
| | GSNQPLRPWYEPGKSFKPSPGNDCTGRGGSVSK | |
| | SLISEPKVVPAIAPFCEGVVKWFNSVKGFGFIET | |
| | KEQRDIFVHFSAIRGEGYKILEPGEKVRFEIGEGR | |
| | KGPQAINVIRIR | |
| SmCas7-11 | MRLKINIHFLEPFRLIEWHEQDRRNKGNSRWQR | 1575 |
| (SEQ ID NO: 16) | GQSFARWHRRKDNDQGRPYITGTLLRSVVIRAV | |
| | EEELARPDTAWQSCGGLFITPDGQTKPQHLRHR | |
| | ATVRARQTAKDKCADRQSACPFCLLLGRFDQV | |
| | GKDGDKKGEGLRFDVRFSNLDLPKDFSPRDFDG | |
| | PQEIGSRRTINRVDDETGKAHDFFSIWEVDAVRE | |
| | FQGEIVLAADLPSRDQVESLLHHALGFVDRLCG | |
| | ARCVISIADQKPAEREERTVAAGDEKATIADYD | |
| | QVKGLPYTRLRPLADAVRNLRQLDLAELNKPD | |
| | GKFLPPGRVNKDGRRVPHYVWDIPLGKGDTLR | |
| | KRLEFLAASCEGDQAKWRNICESEGQALYEKSK | |
| | KLKDSPAAPGRHLGAAEQVRPPQPPVSYSEESIN | |
| | SDLPLAEWIITGTLRAETPFAIGMDAPIDDDQTSS | |
| | RTLVDRDGRYRLPRSTLRGILRRDLSLASGDQG | |
| | CQVRLGPERPCTCPVCLILRQVVIADTVSETTVP | |
| | ADIRQRIRRNPITGTAADGGLFDTERGPKGAGFP | |
| | FSLRYRGHAPMPKALRTVLQWWSAGKCFAGSD | |
| | GGVGCGRFALDNLEVYRWDLGTFAFRQAYSEN | |
| | NGLRSPEEEFDLAVIHELAEGLAKEDGQKILKGT | |
| | EPFTCWQERSWQFSFTGPLLQGDPLAALNSDTA | |
| | DIISFRRTVVDNGEVLREPVLRGEGLRGLLRTAV | |
| | GRVAGDDLLTRSHQDCKCEICQLFGSEHRAGIL | |
| | RFEDLPPVSPTTVADKRLDHVAIDRFDQSVVEK | |
| | YDDRPLVGSPKQPLVFKGCFWVQTSGMTHQLT | |
| | ELLAQAWRDIAAGHYPVGGKGGIGYGWINSLV | |
| | VDGEKITCRPDGDSISLTTVTGDIPPRPALTPPAG | |
| | AIYYPHYFLPPNPEHKPKRSDKIIGHHTFATDPD | |
| | SFTGRITCKLEVVTPLIVPDTEGEQPKDQHKNFP | |
| | FFKINDEIMLPGAPLWAAVSQVYEALTNSCFRV | |
| | MKQKRFLSWRMEAEDYKDFYPGRVLDGGKQI | |
| | KKMGDKAIRMPLYDDSTATGSIKDDQLISDCCP | |
| | KSDEKLQKALATNQKIALAAKHNQEYLAQLSP | |
| | DEREEALQGLKKVSFWTESLANNEAPPFLIAKL | |
| | GEERGKPKRAGYLKITGPNNANIANTNNPDDGG | |
| | YIPSWKDQFDYSFRLLGPPRCLPNTKGNREYPRP | |
| | GFTCVIDGKEYSLTKRCERIFEDISGGENQVVRA | |
| | VTERVREQYREILASYRANAAGIAEGFRTRMYD | |
| | TEELRENDLVYFKTAKQADGKERVVAISPVCIS | |
| | READDRPLGKRLPAGFQPCSHVCLEDCNTCSAK | |
| | NCPVPLYREGWPVNGLCPACRLFGAQMYKGRV | |
| | NFGFARLPDDKQPETKTLTLPLLERPRPTWVLP | |
| | KSVKGSNTEDATIPGRKFYLRHDGWRIVMAGT | |
| | NPITGESIEKTANNATVEAIMPGATFTFDIVCENL | |
| | DQQELGLLLYSLELEEGMSHTLGRGKPLGFGNV | |
| | RIKVEKIEKRLSDGSRREMIPPKGAGLFMTDKV | |
| | QDALRGLTEGGDWHQRPHISGLRRLLTRYPEIK | |
| | ARYPKLSQGEDKEPGYIELKSQKDENGVPIYNP | |
| | NRELRVSENGPLPWFLLAKK | |
| SstCas7-11 | MIINITVKFLGPFRMLEWTDPDNRNRKNREFMR | 1652 |
| (SEQ ID NO: 17) | GQAFARWHNSNPQKGSQPYITGTLVRSAVIRSA | |
| | ENLLMLSEGKVGKEKCCPGEFRTENRKKRDAM | |
| | LHLRQRSTLQWKTDKPLCNGKSLCPICELLGRRI | |
| | GKTDEVKKKGDFRIHFGNLTPLNRYDDPSDIGT | |
| | QRTLNRVDYATGKAHDFFKVWEIDHSLLSVFQ | |
| | GKISIADNIGDGATKLLEDSLRFTDRLCGAICVIS | |
| | YDCIENSDGKENGKTGEAAHIMGESDAGKTDA | |
| | ENIANAIADMMGTAGEPEKLRILADAVRALRIG | |
| | KNTVSQLPLDHEGKENHHLWDIGEGKSIRELLL | |
| | EKAESLPSDQWRKFCEDVGEILYLKSKDPTGGL | |
| | TVSQRILGDEAFWSKADRQLNPSAVSIPVTTETL | |
| | ICGKLISETPFFFGTEIEDAKHTNLKVLLDRQNR | |
| | YRLPRSAIRGVLRRDLRTAFGGKGCNVELGGRP | |
| | CLCDVCRIMRGITIMDARSEYAEPPEIRHRIRLNP | |
| | YTGTVAEGALFDMELGPQGLSFDFILRYRGKGK | |
| | SIPKALRNVLKWWTKGQAFLSGAASTGKGIFRL | |
| | DDLKYISFDLSDKDKRKDYLDNYGWRNRIEALS | |
| | LEKMPLDRMNDYAEPLWQKVSVEIEIGSPFLNG | |
| | DPIRALIEKDGSDIVSFRKYADDSGKEVYAYKA | |
| | ESFRGVVRAALARQHFDKEGKPLDKEGKPLLTL | |
| | IHQDCECLICRLFGSEHETGRLRFEDLLFDPQPEP | |
| | MIFDHVAIDRFTGGAVDKKKFDDCSLPGTPGHP | |
| | LTLKGCFWIRKELEKPDEDKSEREALSKALADI | |
| | HNGLYPLGGKGAIGYGQVMNLKIKGAGDVIKA | |
| | ALQSESSRMSASEPEHKKPDSGLKLSFDDKKAV | |
| | YYPHYFLKPAAEEVNRKPIPTGHETLNSGLLTG | |
| | KIRCRLTTRTPLIVPDTSNDDFFQTGVEGHESYA | |
| | FFSVNGDIMLPGSEIRGMLSSVYEALTNSCFRVF | |
| | DEGYRLSWRMEADRNVLMQFKPGRVTDNGLRI | |
| | EEMKEYRYPFYDRDCSDKKSQEAYFDEWERSIT | |
| | LTDDSLEKMAERKGDISPKDLKVLKSLKGKNY | |
| | KSTEGLLAAFKDKGGDTGGNILGLIFKYAERIG | |
| | DVPRYEHPTDTDRMMLSLSEYNRNQKSDGKRA | |
| | YKIIKPASKLGKGAYFMFAGTSVENKRICNPAC | |
| | TDKANKSVKGYLKISGPNKLEKYNISEPELDGV | |
| | PEDRNCQIIHNRIYLRKIFVANAKKRKERDRLVG | |
| | EFACYDPEKKVTYSMTKRCERIFIKDRGRTLPIT | |
| | HEASELFEILVQEYRENAKRQDTPEVFQTLLPDN | |
| | GRLNPGDLVYFREEKGKTVEIIPVRISRKIDDSPI | |
| | GKRLREDLRPCHGEWIEGDDLSQLSEYPEKKLF | |
| | TRNTEGLCPACRLFGTGAYKGRLRFGFAKLEND | |
| | PKWLMKNSDGPSHGGPLTLPLLERPRPTWSMP | |
| | DDTLNRLKKDGKQEPKKQKGKKGPQVPGRKFY | |
| | VHHDGWKEINCGCHPTTKENIVQNQNNRTVEP | |
| | LDKGNTFSFEICFENLEPYELGLLLYTLELEKGL | |
| | AHKLGMAKPMGFGSIDIEVENVSLRTDSGQWK | |
| | DANEQISEWTDKGKKDAGKWFKTDWEAAEHI | |
| | KNLKKLLFLPGEEQNPRVIYPALKQKDIPNSRLP | |
| | GYEELKKNLNMEKRKEMLTTPWAPWHPIKK | |
| SybCas7-11 | MFPKGRQMRRQRLLGDAEYYGGTGREQPASIVI | 1329 |
| (SEQ ID NO: 18) | STDSDPDHKVYEWIITGQLKAETGFFFGTKAGA | |
| | GGHTDLSILLGKDGHYRVPRSVFRGALRRDLRV | |
| | AFGAGCRVEVGRERPCECPVCKVMRQITVMDTI | |
| | SSYREAPEIRQRIRLNPYTGTVDKGALFDMEVG | |
| | PEGIEFPFVLRFRGSKSFPSELAAVIGSWTKGTA | |
| | WLGGAAATGKGRFSLLGLSIHKWNLSTAEGRK | |
| | SYLAAYGLRDAADKTVKRLSIDKGGKGDVGLP | |
| | AGLERDALPSSVREPLWKKLVCTVDFSSPLLLA | |
| | DPIAALLGVEGDERIGFDNIAYEKRRYNGETNTT | |
| | ESIPAVKGETFRGIVRTALGKRHGNLTRDHEDC | |
| | RCRLCAVFGKEQEAGKIRFEDLMPVGAWTRKH | |
| | LDHVAIDRFHGGAEENMKFDTYALAASPTNPLR | |
| | MKGLIWVRSDLFETGHDGPTPPYVKDIIDALAD | |
| | VKRGLYPVGGKTGSGYGWIKDVTIDGLPQGLSL | |
| | PPAEERVDGVNEVPPYNYSAPPDLPSAAEGEYF | |
| | FPHVFIKPYDKVDRVSRLTGHDRFRQGRITGRIT | |
| | CTLKTLTPLIIPDSEGIQTDATGHKMCKFFSVAG | |
| | KPMIPGSEIRGMISSVYEALTNSCFRVFDEEKYL | |
| | TRRVQPKKGAKSSELVPGIIVWGQNGGLAVQQ | |
| | VKNAYRVPLYDDPAVTSAIPTEAQKNKERWES | |
| | VPSVNLQGALDWNLTTANIARDNRTFLNSRPEE | |
| | KDAILSGTKPISFELEGTNPNDMLVRLVPDGVD | |
| | GAHSGYLKFTGLNMVLKANKKTSRKLAPSEED | |
| | VRTLAILHNDFDSRRDWRRPPNSQRYFPRSVLR | |
| | FSLERSTYTIPKRCERVFEGTCGEPYSVPSDVER | |
| | QYNSIIDDISKNYGRISETYLTKTANRKLTVGDL | |
| | VYFIADLDKNMATHILPVFISRISDEKPLGELLPF | |
| | SGKLIPCEGEPPTILKKMAPSLLTEAWRTLISTHL | |
| | EGFCPACRLFGTTSYKGRIRFGFAEHTGTPKWL | |
| | REELDWARPFLTLPIQERPRPTWSVPDDKSEVPG | |
| | RKFYLHHHGGNRIVESNLRNRPEVNQTKNNSSV | |
| | EPISAGNTFTFDVCFENLEAWELGLLLYCLELSP | |
| | KLAHKLGRAKAFGFGSVKIHVERIEERTTDGAY | |
| | QDVTAVKKNGWITTGHDKLREWFHRDDWEDV | |
| | DHIRNLRTVLRFPDADQEHDVRYPELKANNGVS | |
| | GYVELRDKMTASERQESLRTPWYRWFPQNGTG | |
| | GSGRHEQAATSQEQDTAKDESVLSATQRRQAVI | |
| | DVSDPDERLSGTVESFDRQKGDGYIGCGVRQFY | |
| | VRLEDIRSRTALCEGQVVTFRARKEWEGHEAY | |
| | DVEIDQ | |

TABLE 2
Sequences of synthetic Cas7-S effector proteins. Synthetic proteins
can have different domain and linker substitutions. The synthetic proteins of this table are
comprised of different domains from Cas7-11 and Cas7-1 effectors. L1, L2, L3, and L4 refer
to linker domains derived from Cas7-11 and Cas7-1 effectors. Cas7-11 effectors include linkers
L1-L4, while Cas7-1 include only linkers L3 and L4. FIG. 17 indicates the domain boundaries
of the synthetic Cas7-1 effector proteins included in this table.

| | | | Domain |
|---|---|---|---|
| Name | | | Breakdown of |
| (SEQ ID | | Length | Synthetic Proteins |
| NO: ) | Amino Acid Sequence (N→C) | (Amino Acids) | (indicates origin) |
| Cas7-S3 | MTTTMKISIEFLEPFRMTKWQESTRRNKNN | 1336 | Cas7.1: DiCas7-11 |
| (SEQ ID | KEFVRGQAFARWHRNKKDNTKGRPYITGT | | L1: DiCas7-11 |
| NO: 19) | LLRSAVIRSAENLLTLSDGKISEKTCCPGKF | | L2: DiCas7-11 |
| | DTEDKDRLLQLRQRSTLRWTDKNPCPDNA | | Cas7.2: Cas7-1 and |
| | ETYCPFCELLGRSGNDGKKAEKKDWRFRIH | | DiCas7-11 |
| | FGNLSLPGKPDFDGPKAIGSQRVLNRVDFK | | L3: Cas7-1 |
| | SGKAHDFFKAYEVDHTRFPRFEGEITIDNK | | Cas7.3: Cas7-1 |
| | VSAEARKLLCDSLKFTDRLCGALCVIRFDE | | L4: Cas7-1 |
| | YTPAADSGKQTENVQAEPNAMSGGLKITR | | Cas7.4: Cas7-1 |
| | RILGDAEFHGKPDRLEKSRSVSIGSVLKETV | | INS: Cas7-1 |
| | VCGELVAKTPFFFGAIDEDAKQTDLQVLLT | | CTE: Cas7-1 |
| | PDNKYRLPRSAVRGILRRDLQTYFDSPCNA | | Cas1: Cas7-1 |
| | ELGGRPCMCKTCRIMRGITVMDARSEYNA | | |
| | PPEIRHRTRINPFTGTAKLLCLDLEVGPEGIV | | |
| | FPFVMRYRGQGEKLPSELTDVIHLWKEEM | | |
| | AFLGGSASTGKGRFSLEPLDIFQWDLTDPGS | | |
| | LKDYIECSGLRRPEEELEEWSEALQGLNPFS | | |
| | LPDTSDKYSPQWTKVGYEIKIQSPLLSNDPI | | |
| | AALFDTANHDAVAYKKRVWENGRIIERHT | | |
| | FKGESIRGVLRTALGREYGLFELKHEDCPCS | | |
| | LCTIFGNEHEAGKIRFEDLEIEDGVAEKHFD | | |
| | HVAIDRFTGGAVDKKKFDDFSLAGSPGKPI | | |
| | KLKGCFWLRADIFSDEKKDEKKKIINALCD | | |
| | VRDGLYPLGGKGGVGYGWVKELRITDGPD | | |
| | DFKLPEPKCKEIPPDGTPSDYPFPQLKPLQT | | |
| | EKDKVYSPHYFLKPAEKKVDRNWEKRLIG | | |
| | HERFDKDLLTGKIECTLKTLSPLIIPDSEVVT | | |
| | ENGNGHKSYQFFRLNNEIIIPGSEIRGMISSV | | |
| | YEALTNSCFRVVDDIIPVVISRSVSPYQIAQK | | |
| | LPSPDLRPCEREILEDSEIGSLDKCSEKRLFR | | |
| | LHEDGLCPSCRLFGTTHYKGRVRFGFAKHE | | |
| | GGEKWLMDDKTKDEYLTLPLLERPRPTWS | | |
| | MPSDEDDVPGRKFYVHHNGWEKVKQDSL | | |
| | DNKTSKTENNRSVEPLDKDNLFKFDVFFEN | | |
| | LEPWELGLLLYSLELEEGLAHKFGMAKAF | | |
| | GFGSTKIDADKILLREAPNEWQDATNRKDD | | |
| | MLRKGFGELTSWFDKDWDKVEHIDGLRSL | | |
| | LQIPEALSRAEVRYPDLTEDDPEKPPGYVQL | | |
| | SDKKEGRKYLIRPWQPWYPMLKSITTTESL | | |
| | LTRFLSKANFLSAYKRIAVKKAAGDLVNSA | | |
| | LNYGYGILYGRSINAIIQAGLNPMAGFLHSY | | |
| | QPTKPVLVYDLVEEFRPLAVDRGIFTLLNR | | |
| | GQKIELGRDSLLSAKSRKKIAKSVIGCLSSE | | |
| | VCFNGHRLSLEDVIKEQALNIKKHLYNNAK | | |
| | YRPFLGRW* | | |
| Cas7-S7 | MTTTMKISIEFLEPFRMTKWQESTRRNKNN | 1665 | Cas7.1: DiCas7-11 |
| (SEQ ID | KEFVRGQAFARWHRNKKDNTKGRPYITGT | | L1: DiCas7-11 |
| NO: 20) | LLRSAVIRSAENLLTLSDGKISEKTCCPGKF | | L2: DiCas7-11 |
| | DTEDKDRLLQLRQRSTLRWTDKNPCPDNA | | Cas7.2: DiCas7-11 |
| | ETYCPFCELLGRSGNDGKKAEKKDWRFRIH | | L3: DiCas7-11 |
| | FGNLSLPGKPDFDGPKAIGSQRVLNRVDFK | | Cas7.3: DiCas7-11 |
| | SGKAHDFFKAYEVDHTRFPRFEGEITIDNK | | L4: DiCas7-11 |
| | VSAEARKLLCDSLKFTDRLCGALCVIRFDE | | Cas7.4: DiCas7-11 |
| | YTPAADSGKQTENVQAEPNAMSGGLKITR | | INS: DiCas7-11 |
| | RILGDAEFHGKPDRLEKSRSVSIGSVLKETV | | CTE: Cas7-1 |
| | VCGELVAKTPFFFGAIDEDAKQTDLQVLLT | | Cas1: Cas7-1 |
| | PDNKYRLPRSAVRGILRRDLQTYFDSPCNA | | |
| | ELGGRPCMCKTCRIMRGITVMDARSEYNA | | |
| | PPEIRHRTRINPFTGTVAEGALFNMEVAPEG | | |
| | IVFPFQLRYRGSEDGLPDALKTVLKWWAE | | |
| | GQAFMSGAASTGKGRFRMENAKYETLDLS | | |
| | DENQRNDYLKNWGWRDEKGLEELKKRLN | | |
| | SGLPEPGNYRDPKWHEINVSIEMASPFINGD | | |
| | PIRAAVDKRGTDVVTFVKYKAEGEEAKPV | | |
| | CAYKAESFRGVIRSAVARIHMEDGVPLTEL | | |
| | THSDCECLLCQIFGSEYEAGKIRFEDLVFES | | |
| | DPEPVTFDHVAIDRFTGGAADKKKFDDSPL | | |
| | PGSPARPLMLKGSFWIRRDVLEDEEYCKAL | | |
| | GKALADVNNGLYPLGGKSAIGYGQVKSLG | | |
| | IKGDDKRISRLMNPAFDETDVAVPEKPKTD | | |
| | AEVRIEAEKVYYPHYFVEPHKKVEREEKPC | | |
| | GHQKFHEGRLTGKIRCKLITKTPLIVPDTSN | | |
| | DDFFRPADKEARKEKDEYHKSYAFFRLHK | | |
| | QIMIPGSELRGMVSSVYETVINSCFRIFDET | | |
| | KRLSWRMDADHQNVLQDFLPGRVTADGK | | |
| | HIQKFSETARVPFYDKTQKHFDILDEQEIAG | | |
| | EKPVRMWVKRFIKRLSLVDPAKHPQKKQD | | |
| | NKWKRRKEGIATFIEQKNGSYYFNVVINN | | |
| | GCTSFHLWHKPDNFDQEKLEGIQNGEKLD | | |
| | CWVRDSRYQKAFQEIPENDPDGWECKEGY | | |
| | LHVVGPSKVEFSDKKGDVINNFQGTLPSVP | | |
| | NDWKTIRTNDFKNRKRKNEPVFCCEDDKG | | |
| | NYYTMAKYCETFFFDLKENEEYEIPEKARI | | |
| | KYKELLRVYNNNPQAVPESVFQSRVAREN | | |
| | VEKLKSGDLVYFKHNEKYVEDIVPVRISRT | | |
| | VDDRMIGKRMSADLRPCHGDWVEDGDLS | | |
| | ALNAYPEKRLLLRHPKGLCPACRLFGTGSY | | |
| | KGRVRFGFASLENDPEWLIPGKNPGDPFHG | | |
| | GPVMLSLLERPRPTWSIPGSDNKFKVPGRK | | |
| | FYVHHHAWKTIKDGNHPTTGKAIEQSPNN | | |
| | RTVEALAGGNSFSFEIAFENLKEWELGLLIH | | |
| | SLQLEKGLAHKLGMAKSMGFGSVEIDVES | | |
| | VRLREAPNEWQDATNRKDDMLRKGFGELT | | |
| | SWFDKDWDKVEHIDGLRSLLQIPEALSRAE | | |
| | VRYPDLTEDDPEKPPGYVQLSDKKEGRKY | | |
| | LIRPWQPWYPMLKSITTTESLLTRFLSKANF | | |
| | LSAYKRIAVKKAAGDLVNSALNYGYGILY | | |
| | GRSINAIIQAGLNPMAGFLHSYQPTKPVLVY | | |
| | DLVEEFRPLAVDRGIFTLLNRGQKIELGRDS | | |
| | LLSAKSRKKIAKSVIGCLSSEVCFNGHRLSL | | |
| | EDVIKEQALNIKKHLYNNAKYRPFLGRW* | | |
| Cas7-S10 | MTTTMKISIEFLEPFRMTKWQESTRRNKNN | 1266 | Cas7.1: DiCas7-11 |
| (SEQ ID | KEFVRGQAFARWHRNKKDNTKGRPYITGT | | L1: DiCas7-11 |
| NO: 21) | LLRSAVIRSAENLLTLSDGKISEKTCCPGKF | | Cas11:: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: Cas7-1 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: Cas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWT | | |
| | KVGYEIKIQSPLLSNDPIAALFDTANHDAVA | | |
| | YKKRVWENGRIIERHTFKGESIRGVLRTAL | | |
| | GREYGLFELKHEDCPCSLCTIFGNEHEAGKI | | |
| | RFEDLEIEDGVAEKHFDHVAIDRFTGGAVD | | |
| | KKKFDDFSLAGSPGKPIKLKGCFWLRADIF | | |
| | SDEKKDEKKKIINALCDVRDGLYPLGGKGG | | |
| | VGYGWVKELRITDGPDDFKLPEPKCKEIPP | | |
| | DGTPSDYPFPQLKPLQTEKDKVYSPHYFLK | | |
| | PAEKKVDRNWEKRLIGHERFDKDLLTGKIE | | |
| | CTLKTLSPLIIPDSEVVTENGNGHKSYQFFR | | |
| | LNNEIIIPGSEIRGMISSVYEALTNSCFRVVD | | |
| | DIIPVVISRSVSPYQIAQKLPSPDLRPCEREIL | | |
| | EDSEIGSLDKCSEKRLFRLHEDGLCPSCRLF | | |
| | GTTHYKGRVRFGFAKHEGGEKWLMDDKT | | |
| | KDEYLTLPLLERPRPTWSMPSDEDDVPGRK | | |
| | FYVHHNGWEKVKQDSLDNKTSKTENNRSV | | |
| | EPLDKDNLFKFDVFFENLEPWELGLLLYSL | | |
| | ELEEGLAHKFGMAKAFGFGSTKIDADKILL | | |
| | REAPNEWQDATNRKDDMLRKGFGELTSW | | |
| | FDKDWDKVEHIDGLRSLLQIPEALSRAEVR | | |
| | YPDLTEDDPEKPPGYVQLSDKKEGRKYLIR | | |
| | PWQPWY* | | |
| Cas7-S11 | MTTTMKISIEFLEPFRMTKWQESTRRNKNN | 1264 | Cas7.1: DiCas7-11 |
| (SEQ ID | KEFVRGQAFARWHRNKKDNTKGRPYITGT | | L1: DiCas7-11 |
| NO: 22) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: Cas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWH | | |
| | EINVSIEMASPFINGDPIRAAVDKRGTDVVT | | |
| | FVKYKAEGEEAKPVCAYKAESFRGVIRSAV | | |
| | ARIHMEDGVPLTELTHSDCECLLCQIFGSEY | | |
| | EAGKIRFEDLVFESDPEPVTFDHVAIDRFTG | | |
| | GAADKKKFDDSPLPGSPARPLMLKGSFWIR | | |
| | RDVLEDEEYCKALGKALADVNNGLYPLGG | | |
| | KSAIGYGQVKSLGIKGDDKRISRLMNPAFD | | |
| | ETDVAVPEKPKTDAEVRIEAEKVYYPHYFV | | |
| | EPHKKVEREEKPCGHQKFHEGRLTGKIECT | | |
| | LKTLSPLIIPDSEVVTENGNGHKSYQFFRLN | | |
| | NEIIIPGSEIRGMISSVYEALTNSCFRVVDDII | | |
| | PVVISRSVSPYQIAQKLPSPDLRPCEREILED | | |
| | SEIGSLDKCSEKRLFRLHEDGLCPSCRLFGT | | |
| | THYKGRVRFGFAKHEGGEKWLMDDKTKD | | |
| | EYLTLPLLERPRPTWSMPSDEDDVPGRKFY | | |
| | VHHNGWEKVKQDSLDNKTSKTENNRSVEP | | |
| | LDKDNLFKFDVFFENLEPWELGLLLYSLEL | | |
| | EEGLAHKFGMAKAFGFGSTKIDADKILLRE | | |
| | APNEWQDATNRKDDMLRKGFGELTSWFD | | |
| | KDWDKVEHIDGLRSLLQIPEALSRAEVRYP | | |
| | DLTEDDPEKPPGYVQLSDKKEGRKYLIRPW | | |
| | QPWY* | | |
| Cas7-S12 | MTTTMKISIEFLEPFRMTKWQESTRRNKNN | 1290 | Cas7.1: DiCas7-11 |
| (SEQ ID | KEFVRGQAFARWHRNKKDNTKGRPYITGT | | L1: DiCas7-11 |
| NO: 23) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: DiCas7-11 |
| | ISILDDNKKTEYTRLLADAIRSLRRSSKLVA | | INS: Cas7-1 |
| | GLPKDHDGKDDHYLWDIGKKKKDENSVTI | | CTE: DiCas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWH | | |
| | EINVSIEMASPFINGDPIRAAVDKRGTDVVT | | |
| | FVKYKAEGEEAKPVCAYKAESFRGVIRSAV | | |
| | ARIHMEDGVPLTELTHSDCECLLCQIFGSEY | | |
| | EAGKIRFEDLVFESDPEPVTFDHVAIDRFTG | | |
| | GAADKKKFDDSPLPGSPARPLMLKGSFWIR | | |
| | RDVLEDEEYCKALGKALADVNNGLYPLGG | | |
| | KSAIGYGQVKSLGIKGDDKRISRLMNPAFD | | |
| | ETDVAVPEKPKTDAEVRIEAEKVYYPHYFV | | |
| | EPHKKVEREEKPCGHQKFHEGRLTGKIRCK | | |
| | LITKTPLIVPDTSNDDFFRPADKEARKEKDE | | |
| | YHKSYAFFRLHKQIMIPGSELRGMVSSVYE | | |
| | TVTNSCFRIFDETKRLSWRRSVSPYRTVDD | | |
| | RMIGKRMSADLRPCHGDWVEDGDLSALN | | |
| | AYPEKRLLLRHPKGLCPACRLFGTGSYKGR | | |
| | VRFGFASLENDPEWLIPGKNPGDPFHGGPV | | |
| | MLSLLERPRPTWSIPGSDNKFKVPGRKFYV | | |
| | HHHAWKTIKDGNHPTTGKAIEQSPNNRTVE | | |
| | ALAGGNSFSFEIAFENLKEWELGLLIHSLQL | | |
| | EKGLAHKLGMAKSMGFGSVEIDVESVRLR | | |
| | EAPNEWQDATNRKDDMLRKGFGELTSWF | | |
| | DKDWDKVEHIDGLRSLLQIPEALSRAEVRY | | |
| | PDLTEDDPEKPPGYVQLSDKKEGRKYLIRP | | |
| | WQPWY* | | |
| Cas7-S13 | MTTTMKISIEFLEPFRMTKWQESTRRNKNN | 1667 | Cas7.1: DiCas7-11 |
| (SEQ ID | KEFVRGQAFARWHRNKKDNTKGRPYITGT | | L1: DiCas7-11 |
| NO: 24) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAPMLKSITTTE | | Cas7.4: DiCas7-11 |
| | SLLTRFLSKANFLSAYKRIAVKKAAGDLVN | | INS: DiCas7-11 |
| | SALNYGYGILYGRSINAIIQAGLNPMAGFLH | | CTE: DiCas7-11 |
| | SYQPTKPVLVYDLVEEFRPLAVDRGIFTLLN | | |
| | RGQKIELGRDSLLSAKSRKKIAKSVIGCLSS | | |
| | EVCFNGHRLSLEDVIKEQALNIKKHLYNNA | | |
| | KYRPFLGRWMSGGLKITRRILGDAEFHGKP | | |
| | DRLEKSRSVSIGSVLKETVVCGELVAKTPFF | | |
| | FGAIDEDAKQTDLQVLLTPDNKYRLPRSAV | | |
| | RGILRRDLQTYFDSPCNAELGGRPCMCKTC | | |
| | RIMRGITVMDARSEYNAPPEIRHRTRINPFT | | |
| | GTVAEGALFNMEVAPEGIVFPFQLRYRGSE | | |
| | DGLPDALKTVLKWWAEGQAFMSGAASTG | | |
| | KGRFRMENAKYETLDLSDENQRNDYLKN | | |
| | WGWRDEKGLEELKKRLNSGLPEPGNYRDP | | |
| | KWHEINVSIEMASPFINGDPIRAAVDKRGT | | |
| | DVVTFVKYKAEGEEAKPVCAYKAESFRGV | | |
| | IRSAVARIHMEDGVPLTELTHSDCECLLCQI | | |
| | FGSEYEAGKIRFEDLVFESDPEPVTFDHVAI | | |
| | DRFTGGAADKKKFDDSPLPGSPARPLMLK | | |
| | GSFWIRRDVLEDEEYCKALGKALADVNNG | | |
| | LYPLGGKSAIGYGQVKSLGIKGDDKRISRL | | |
| | MNPAFDETDVAVPEKPKTDAEVRIEAEKV | | |
| | YYPHYFVEPHKKVEREEKPCGHQKFHEGR | | |
| | LTGKIRCKLITKTPLIVPDTSNDDFFRPADKE | | |
| | ARKEKDEYHKSYAFFRLHKQIMIPGSELRG | | |
| | MVSSVYETVTNSCFRIFDETKRLSWRMDA | | |
| | DHQNVLQDFLPGRVTADGKHIQKFSETAR | | |
| | VPFYDKTQKHFDILDEQEIAGEKPVRMWV | | |
| | KRFIKRLSLVDPAKHPQKKQDNKWKRRKE | | |
| | GIATFIEQKNGSYYFNVVTNNGCTSFHLWH | | |
| | KPDNFDQEKLEGIQNGEKLDCWVRDSRYQ | | |
| | KAFQEIPENDPDGWECKEGYLHVVGPSKV | | |
| | EFSDKKGDVINNFQGTLPSVPNDWKTIRTN | | |
| | DFKNRKRKNEPVFCCEDDKGNYYTMAKY | | |
| | CETFFFDLKENEEYEIPEKARIKYKELLRVY | | |
| | NNNPQAVPESVFQSRVARENVEKLKSGDL | | |
| | VYFKHNEKYVEDIVPVRISRTVDDRMIGKR | | |
| | MSADLRPCHGDWVEDGDLSALNAYPEKRL | | |
| | LLRHPKGLCPACRLFGTGSYKGRVRFGFAS | | |
| | LENDPEWLIPGKNPGDPFHGGPVMLSLLER | | |
| | PRPTWSIPGSDNKFKVPGRKFYVHHHAWK | | |
| | TIKDGNHPTTGKAIEQSPNNRTVEALAGGN | | |
| | SFSFEIAFENLKEWELGLLIHSLQLEKGLAH | | |
| | KLGMAKSMGFGSVEIDVESVRLRKDWKQ | | |
| | WRNGNSEIPNWLGKGFAKLKEWFRDELDF | | |
| | IENLKKLLWFPEGDQAPRVCYPMLRKKDD | | |
| | PNGNSGYEELKDGEFKKEDRQKKLTTPWT | | |
| | PWA* | | |
| Cas7-S14 | MTTTMKISIEFLEPFRMTKWQESTRRNKNN | 1358 | Cas7.1: DiCas7-11 |
| (SEQ ID | KEFVRGQAFARWHRNKKDNTKGRPYITGT | | L1: DiCas7-11 |
| NO: 25) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAPMLKSITTTE | | Cas7.4: DiCas7-11 |
| | SLLTRFLSKANFLSAYKRIAVKKAAGDLVN | | INS: Cas7-1 |
| | SALNYGYGILYGRSINAIIQAGLNPMAGFLH | | CTE: DiCas7-11 |
| | SYQPTKPVLVYDLVEEFRPLAVDRGIFTLLN | | |
| | RGQKIELGRDSLLSAKSRKKIAKSVIGCLSS | | |
| | EVCFNGHRLSLEDVIKEQALNIKKHLYNNA | | |
| | KYRPFLGRWMSGGLKITRRILGDAEFHGKP | | |
| | DRLEKSRSVSIGSVLKETVVCGELVAKTPFF | | |
| | FGAIDEDAKQTDLQVLLTPDNKYRLPRSAV | | |
| | RGILRRDLQTYFDSPCNAELGGRPCMCKTC | | |
| | RIMRGITVMDARSEYNAPPEIRHRTRINPFT | | |
| | GTVAEGALFNMEVAPEGIVFPFQLRYRGSE | | |
| | DGLPDALKTVLKWWAEGQAFMSGAASTG | | |
| | KGRFRMENAKYETLDLSDENQRNDYLKN | | |
| | WGWRDEKGLEELKKRLNSGLPEPGNYRDP | | |
| | KWHEINVSIEMASPFINGDPIRAAVDKRGT | | |
| | DVVTFVKYKAEGEEAKPVCAYKAESFRGV | | |
| | IRSAVARIHMEDGVPLTELTHSDCECLLCQI | | |
| | FGSEYEAGKIRFEDLVFESDPEPVTFDHVAI | | |
| | DRFTGGAADKKKFDDSPLPGSPARPLMLK | | |
| | GSFWIRRDVLEDEEYCKALGKALADVNNG | | |
| | LYPLGGKSAIGYGQVKSLGIKGDDKRISRL | | |
| | MNPAFDETDVAVPEKPKTDAEVRIEAEKV | | |
| | YYPHYFVEPHKKVEREEKPCGHQKFHEGR | | |
| | LTGKIRCKLITKTPLIVPDTSNDDFFRPADKE | | |
| | ARKEKDEYHKSYAFFRLHKQIMIPGSELRG | | |
| | MVSSVYETVTNSCFRIFDETKRLSWRRSVS | | |
| | PYRTVDDRMIGKRMSADLRPCHGDWVED | | |
| | GDLSALNAYPEKRLLLRHPKGLCPACRLFG | | |
| | TGSYKGRVRFGFASLENDPEWLIPGKNPGD | | |
| | PFHGGPVMLSLLERPRPTWSIPGSDNKFKV | | |
| | PGRKFYVHHHAWKTIKDGNHPTTGKAIEQ | | |
| | SPNNRTVEALAGGNSFSFEIAFENLKEWEL | | |
| | GLLIHSLQLEKGLAHKLGMAKSMGFGSVEI | | |
| | DVESVRLRKDWKQWRNGNSEIPNWLGKG | | |
| | FAKLKEWFRDELDFIENLKKLLWFPEGDQA | | |
| | PRVCYPMLRKKDDPNGNSGYEELKDGEFK | | |
| | KEDRQKKLTTPWTPWA | | |
| Cas7-S15 | MTTTMKISIEFLEPFRMTKWQESTRRNKNN | 1332 | Cas7.1: DiCas7-11 |
| (SEQ ID | KEFVRGQAFARWHRNKKDNTKGRPYITGT | | L1: DiCas7-11 |
| NO: 26) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: Cas7-1 |
| | YTPAADSGKQTENVQAEPNAPMLKSITTTE | | Cas7.4: Cas7-1 |
| | SLLTRFLSKANFLSAYKRIAVKKAAGDLVN | | INS: Cas7-1 |
| | SALNYGYGILYGRSINAIIQAGLNPMAGFLH | | CTE: Cas7-1 |
| | SYQPTKPVLVYDLVEEFRPLAVDRGIFTLLN | | |
| | RGQKIELGRDSLLSAKSRKKIAKSVIGCLSS | | |
| | EVCFNGHRLSLEDVIKEQALNIKKHLYNNA | | |
| | KYRPFLGRWMSGGLKITRRILGDAEFHGKP | | |
| | DRLEKSRSVSIGSVLKETVVCGELVAKTPFF | | |
| | FGAIDEDAKQTDLQVLLTPDNKYRLPRSAV | | |
| | RGILRRDLQTYFDSPCNAELGGRPCMCKTC | | |
| | RIMRGITVMDARSEYNAPPEIRHRTRINPFT | | |
| | GTVAEGALFNMEVAPEGIVFPFQLRYRGSE | | |
| | DGLPDALKTVLKWWAEGQAFMSGAASTG | | |
| | KGRFRMENAKYETLDLSDENQRNDYLKN | | |
| | WGWRDEKGLEELKKRLNSGLPEPGNYRDP | | |
| | KWTKVGYEIKIQSPLLSNDPIAALFDTANH | | |
| | DAVAYKKRVWENGRIIERHTFKGESIRGVL | | |
| | RTALGREYGLFELKHEDCPCSLCTIFGNEHE | | |
| | AGKIRFEDLEIEDGVAEKHFDHVAIDRFTG | | |
| | GAVDKKKFDDFSLAGSPGKPIKLKGCFWLR | | |
| | ADIFSDEKKDEKKKIINALCDVRDGLYPLG | | |
| | GKGGVGYGWVKELRITDGPDDFKLPEPKC | | |
| | KEIPPDGTPSDYPFPQLKPLQTEKDKVYSPH | | |
| | YFLKPAEKKVDRNWEKRLIGHERFDKDLL | | |
| | TGKIECTLKTLSPLIIPDSEVVTENGNGHKS | | |
| | YQFFRLNNEIIIPGSEIRGMISSVYEALTNSCF | | |
| | RVVDDIIPVVISRSVSPYQIAQKLPSPDLRPC | | |
| | EREILEDSEIGSLDKCSEKRLFRLHEDGLCPS | | |
| | CRLFGTTHYKGRVRFGFAKHEGGEKWLM | | |
| | DDKTKDEYLTLPLLERPRPTWSMPSDEDDV | | |
| | PGRKFYVHHNGWEKVKQDSLDNKTSKTEN | | |
| | NRSVEPLDKDNLFKFDVFFENLEPWELGLL | | |
| | LYSLELEEGLAHKFGMAKAFGFGSTKIDAD | | |
| | KILLREAPNEWQDATNRKDDMLRKGFGEL | | |
| | TSWFDKDWDKVEHIDGLRSLLQIPEALSRA | | |
| | EVRYPDLTEDDPEKPPGYVQLSDKKEGRK | | |
| | YLIRPWQPWY* | | |
| Cas7-S15A | MTTTMKISIEFLEPFRMTKWQESTRRNKNN | 1303 | Cas7.1: DiCas7-11 |
| (SEQ ID | KEFVRGQAFARWHRNKKDNTKGRPYITGT | | L1: DiCas7-11 |
| NO: 27) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: Cas7-1 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: Cas7-1 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLKETVVCGELVAK | | |
| | TPFFFGAIDEDAKQTDLQVLLTPDNKYRLP | | |
| | RSAVRGILRRDLQTYFDSPCNAELGGRPCM | | |
| | CKTCRIMRGITVMDARSEYNAPPEIRHRTRI | | |
| | NPFTGTVAEGALFNMEVAPEGIVFPFQLRY | | |
| | RGSEDGLPDALKTVLKWWAEGQAFMSGA | | |
| | ASTGKGRFRMENAKYETLDLSDENQRNDY | | |
| | LKNWGWRDEKGLEELKKRLNSGLPEPGNY | | |
| | RDPKWTKVGYEIKIQSPLLSNDPIAALFDTA | | |
| | NHDAVAYKKRVWENGRIIERHTFKGESIRG | | |
| | VLRTALGREYGLFELKHEDCPCSLCTIFGNE | | |
| | HEAGKIRFEDLEIEDGVAEKHFDHVAIDRFT | | |
| | GGAVDKKKFDDFSLAGSPGKPIKLKGCFW | | |
| | LRADIFSDEKKDEKKKIINALCDVRDGLYPL | | |
| | GGKGGVGYGWVKELRITDGPDDFKLPEPK | | |
| | CKEIPPDGTPSDYPFPQLKPLQTEKDKVYSP | | |
| | HYFLKPAEKKVDRNWEKRLIGHERFDKDL | | |
| | LTGKIECTLKTLSPLIIPDSEVVTENGNGHKS | | |
| | YQFFRLNNEIIIPGSEIRGMISSVYEALTNSCF | | |
| | RVVDDIIPVVISRSVSPYQIAQKLPSPDLRPC | | |
| | EREILEDSEIGSLDKCSEKRLFRLHEDGLCPS | | |
| | CRLFGTTHYKGRVRFGFAKHEGGEKWLM | | |
| | DDKTKDEYLTLPLLERPRPTWSMPSDEDDV | | |
| | PGRKFYVHHNGWEKVKQDSLDNKTSKTEN | | |
| | NRSVEPLDKDNLFKFDVFFENLEPWELGLL | | |
| | LYSLELEEGLAHKFGMAKAFGFGSTKIDAD | | |
| | KILLREAPNEWQDATNRKDDMLRKGFGEL | | |
| | TSWFDKDWDKVEHIDGLRSLLQIPEALSRA | | |
| | EVRYPDLTEDDPEKPPGYVQLSDKKEGRK | | |
| | YLIRPWQPWY* | | |
| Cas7-S15B | MTTTMKISIEFLEPFRMTKWQESTRRNKNN | 1311 | Cas7.1: DiCas7-11 |
| (SEQ ID | KEFVRGQAFARWHRNKKDNTKGRPYITGT | | L1: DiCas7-11 |
| NO: 28) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: Cas7-1 |
| | YTPAADSGKQTENVQAEPNAFLSAYKRIAV | | Cas7.4: Cas7-1 |
| | KKAAGDLVNSALNYGYGILYGRSINAIIQA | | INS: Cas7-1 |
| | GLNPMAGFLHSYQPTKPVLVYDLVEEFRPL | | CTE: Cas7-1 |
| | AVDRGIFTLLNRGQKIELGRDSLLSAKSRK | | |
| | KIAKSVIGCLSSEVCFNGHRLSLEDVIKEQA | | |
| | LNIKKHLYNNAKYRPFLGRWMSGGLKITR | | |
| | RILGDAEFHGKPDRLEKSRSVSIGSVLKETV | | |
| | VCGELVAKTPFFFGAIDEDAKQTDLQVLLT | | |
| | PDNKYRLPRSAVRGILRRDLQTYFDSPCNA | | |
| | ELGGRPCMCKTCRIMRGITVMDARSEYNA | | |
| | PPEIRHRTRINPFTGTVAEGALFNMEVAPEG | | |
| | IVFPFQLRYRGSEDGLPDALKTVLKWWAE | | |
| | GQAFMSGAASTGKGRFRMENAKYETLDLS | | |
| | DENQRNDYLKNWGWRDEKGLEELKKRLN | | |
| | SGLPEPGNYRDPKWTKVGYEIKIQSPLLSN | | |
| | DPIAALFDTANHDAVAYKKRVWENGRIIER | | |
| | HTFKGESIRGVLRTALGREYGLFELKHEDC | | |
| | PCSLCTIFGNEHEAGKIRFEDLEIEDGVAEK | | |
| | HFDHVAIDRFTGGAVDKKKFDDFSLAGSPG | | |
| | KPIKLKGCFWLRADIFSDEKKDEKKKIINAL | | |
| | CDVRDGLYPLGGKGGVGYGWVKELRITDG | | |
| | PDDFKLPEPKCKEIPPDGTPSDYPFPQLKPL | | |
| | QTEKDKVYSPHYFLKPAEKKVDRNWEKRL | | |
| | IGHERFDKDLLTGKIECTLKTLSPLIIPDSEV | | |
| | VTENGNGHKSYQFFRLNNEIIIPGSEIRGMIS | | |
| | SVYEALTNSCFRVVDDIIPVVISRSVSPYQIA | | |
| | QKLPSPDLRPCEREILEDSEIGSLDKCSEKRL | | |
| | FRLHEDGLCPSCRLFGTTHYKGRVRFGFAK | | |
| | HEGGEKWLMDDKTKDEYLTLPLLERPRPT | | |
| | WSMPSDEDDVPGRKFYVHHNGWEKVKQD | | |
| | SLDNKTSKTENNRSVEPLDKDNLFKFDVFF | | |
| | ENLEPWELGLLLYSLELEEGLAHKFGMAK | | |
| | AFGFGSTKIDADKILLREAPNEWQDATNRK | | |
| | DDMLRKGFGELTSWFDKDWDKVEHIDGLR | | |
| | SLLQIPEALSRAEVRYPDLTEDDPEKPPGYV | | |
| | QLSDKKEGRKYLIRPWQPWY* | | |
| Cas7-S35 | MTTTMKISIEFLEPFRMTKWQESTRRNKNN | 1249 | Cas7.1: DiCas7-11 |
| (SEQ ID | KEFVRGQAFARWHRNKKDNTKGRPYITGT | | L1: DiCas7-11 |
| NO: 29) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLYEWIIVGRLIAQTPFHFGDE | | |
| | EKAEGAILLTPDNRFRLPRTALRGILRRDLK | | |
| | LAGASACEVEVGRSEPCPCDVCKIMRRVTL | | |
| | LDTVSEDLRDFLPELRKRIRINPQSGTVAEG | | |
| | ALFDTEVGPEGLSFPFVLRYKCEKLPDSLTT | | |
| | VLCWWQEGLAFLSGESATGKGRFRLEING | | |
| | AFVWDLSDENQRNDYLKNWGWRDEKGLE | | |
| | ELKKRLNSGLPEPGNYRDPKWDLIKYQLNI | | |
| | SSPLLLNDPIRAIALYEGEGKAPDAVFFKKY | | |
| | VFENGKIEEKPCFKAESIRGIFRTAVGRIKN | | |
| | VLTKNHEDCICVLCHLFGNVHETGRLKFED | | |
| | LKIVSGQEEKFFDHVAIDRFLGGAKEKYKF | | |
| | DDKPIIGAPDTPIVLEGKIWVKKDINDEAKE | | |
| | TLSQAFSDINTGIYYLGANGSIGYGWIEEVK | | |
| | ALKDDKRISRLMNPAFDETDVAVPEKPKTD | | |
| | AEVRIEAEKVYYPHYFVEPHKKVEREEKPC | | |
| | GHQKFHEGRLTGKIECTLKTLSPLIIPDSEVV | | |
| | TENGNGHKSYQFFRLNNEIIIPGSEIRGMISS | | |
| | VYEALTNSCFRVVDDIIPVVISRSVSPYQIAQ | | |
| | KLPSPDLRPCEREILEDSEIGSLDKCSEKRLF | | |
| | RLHEDGLCPSCRLFGTTHYKGRVRFGFAKH | | |
| | EGGEKWLMDDKTKDEYLTLPLLERPRPTW | | |
| | SMPSDEDDVPGRKFYVHHNGWEKVKQDS | | |
| | LDNKTSKTENNRSVEPLDKDNLFKFDVFFE | | |
| | NLEPWELGLLLYSLELEEGLAHKFGMAKA | | |
| | FGFGSTKIDADKILLREEAGKFHPCEKADEY | | |
| | LKKGLNKLTSWFGKNEINEHMRNLLLFMT | | |
| | YYQNLPKVKYPDFDGYAKWRCSYVEQDK | | |
| | VEYFQNRWIVAS* | | |
| Cas7-S36 | MTTTMKISIEFLEPFRMTKWQESTRRNKNN | 1249 | Cas7.1: DiCas7-11 |
| (SEQ ID | KEFVRGQAFARWHRNKKDNTKGRPYITGT | | L1: DiCas7-11 |
| NO: 30) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLYEWIIVGRLIAQTPFHFGDE | | |
| | EKAEGAILLTPDNRFRLPRTALRGILRRDLK | | |
| | LAGASACEVEVGRSEPCPCDVCKIMRRVTL | | |
| | LDTVSEDLRDFLPELRKRIRINPQSGTVAEG | | |
| | ALFDTEVGPEGLSFPFVLRYKCEKLPDSLTT | | |
| | VLCWWQEGLAFLSGESATGKGRFRLEING | | |
| | AFVWDLSDENQRNDYLKNWGWRDEKGLE | | |
| | ELKKRLNSGLPEPGNYRDPKWTKVGYEIKI | | |
| | QSPLLSNDPIAALFDTANHDAVAYKKRVW | | |
| | ENGRIIERHTFKGESIRGVLRTALGREYGLF | | |
| | ELKHEDCPCSLCTIFGNEHEAGKIRFEDLEIE | | |
| | DGVAEKHFDHVAIDRFTGGAVDKKKFDDF | | |
| | SLAGSPGKPIKLKGCFWLRADIFSDEKKDE | | |
| | KKKIINALCDVRDGLYPLGGKGGVGYGWV | | |
| | KELRITDDDKRISRLMNPAFDETDVAVPEK | | |
| | PKTDAEVRIEAEKVYYPHYFVEPHKKVERE | | |
| | EKPCGHQKFHEGRLTGKIECTLKTLSPLIIPD | | |
| | SEVVTENGNGHKSYQFFRLNNEIIIPGSEIRG | | |
| | MISSVYEALTNSCFRVVDDIIPVVISRSVSPY | | |
| | QIAQKLPSPDLRPCEREILEDSEIGSLDKCSE | | |
| | KRLFRLHEDGLCPSCRLFGTTHYKGRVRFG | | |
| | FAKHEGGEKWLMDDKTKDEYLTLPLLERP | | |
| | RPTWSMPSDEDDVPGRKFYVHHNGWEKV | | |
| | KQDSLDNKTSKTENNRSVEPLDKDNLFKFD | | |
| | VFFENLEPWELGLLLYSLELEEGLAHKFGM | | |
| | AKAFGFGSTKIDADKILLREEAGKFHPCEK | | |
| | ADEYLKKGLNKLTSWFGKNEINEHMRNLL | | |
| | LFMTYYQNLPKVKYPDFDGYAKWRCSYV | | |
| | EQDKVEYFQNRWIVAS* | | |
| Cas7-S37 | MTTTMKISIEFLEPFRMTKWQESTRRNKNN | 1252 | Cas7.1: DiCas7-11 |
| (SEQ ID | KEFVRGQAFARWHRNKKDNTKGRPYITGT | | L1: DiCas7-11 |
| NO: 31) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLYEWIIVGRLIAQTPFHFGDE | | |
| | EKAEGAILLTPDNRFRLPRTALRGILRRDLK | | |
| | LAGASACEVEVGRSEPCPCDVCKIMRRVTL | | |
| | LDTVSEDLRDFLPELRKRIRINPQSGTVAEG | | |
| | ALFDTEVGPEGLSFPFVLRYKCEKLPDSLTT | | |
| | VLCWWQEGLAFLSGESATGKGRFRLEING | | |
| | AFVWDLSDENQRNDYLKNWGWRDEKGLE | | |
| | ELKKRLNSGLPEPGNYRDPKWHEINVSIEM | | |
| | ASPFINGDPIRAAVDKRGTDVVTFVKYKAE | | |
| | GEEAKPVCAYKAESFRGVIRSAVARIHMED | | |
| | GVPLTELTHSDCECLLCQIFGSEYEAGKIRF | | |
| | EDLVFESDPEPVTFDHVAIDRFTGGAADKK | | |
| | KFDDSPLPGSPARPLMLKGSFWIRRDVLED | | |
| | EEYCKALGKALADVNNGLYPLGGKSAIGY | | |
| | GQVKSLGIKGDDKRISRLMNPAFDETDVAV | | |
| | PEKPKTDAEVRIEAEKVYYPHYFVEPHKKV | | |
| | EREEKPCGHQKFHEGRLTGKIECTLKTLSPL | | |
| | IIPDSEVVTENGNGHKSYQFFRLNNEIIIPGS | | |
| | EIRGMISSVYEALTNSCFRVVDDIIPVVISRS | | |
| | VSPYQIAQKLPSPDLRPCEREILEDSEIGSLD | | |
| | KCSEKRLFRLHEDGLCPSCRLFGTTHYKGR | | |
| | VRFGFAKHEGGEKWLMDDKTKDEYLTLPL | | |
| | LERPRPTWSMPSDEDDVPGRKFYVHHNGW | | |
| | EKVKQDSLDNKTSKTENNRSVEPLDKDNLF | | |
| | KFDVFFENLEPWELGLLLYSLELEEGLAHK | | |
| | FGMAKAFGFGSTKIDADKILLREEAGKFHP | | |
| | CEKADEYLKKGLNKLTSWFGKNEINEHMR | | |
| | NLLLFMTYYQNLPKVKYPDFDGYAKWRCS | | |
| | YVEQDKVEYFQNRWIVAS* | | |
| Cas7-S38 | MTTTMKISIEFLEPFRMTKWQESTRRNKNN | 1252 | Cas7.1: DiCas7-11 |
| (SEQ ID | KEFVRGQAFARWHRNKKDNTKGRPYITGT | | L1: DiCas7-11 |
| NO: 32) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWD | | |
| | LIKYQLNISSPLLLNDPIRAIALYEGEGKAPD | | |
| | AVFFKKYVFENGKIEEKPCFKAESIRGIFRT | | |
| | AVGRIKNVLTKNHEDCICVLCHLFGNVHET | | |
| | GRLKFEDLKIVSGQEEKFFDHVAIDRFLGG | | |
| | AKEKYKFDDKPIIGAPDTPIVLEGKIWVKK | | |
| | DINDEAKETLSQAFSDINTGIYYLGANGSIG | | |
| | YGWIEEVKALKDDKRISRLMNPAFDETDV | | |
| | AVPEKPKTDAEVRIEAEKVYYPHYFVEPHK | | |
| | KVEREEKPCGHQKFHEGRLTGKIECTLKTL | | |
| | SPLIIPDSEVVTENGNGHKSYQFFRLNNEIIIP | | |
| | GSEIRGMISSVYEALTNSCFRVVDDIIPVVIS | | |
| | RSVSPYQIAQKLPSPDLRPCEREILEDSEIGS | | |
| | LDKCSEKRLFRLHEDGLCPSCRLFGTTHYK | | |
| | GRVRFGFAKHEGGEKWLMDDKTKDEYLT | | |
| | LPLLERPRPTWSMPSDEDDVPGRKFYVHH | | |
| | NGWEKVKQDSLDNKTSKTENNRSVEPLDK | | |
| | DNLFKFDVFFENLEPWELGLLLYSLELEEG | | |
| | LAHKFGMAKAFGFGSTKIDADKILLREEAG | | |
| | KFHPCEKADEYLKKGLNKLTSWFGKNEINE | | |
| | HMRNLLLFMTYYQNLPKVKYPDFDGYAK | | |
| | WRCSYVEQDKVEYFQNRWIVAS* | | |
| Cas7-S39 | MTTTMKISIEFLEPFRMTKWQESTRRNKNN | 1252 | Cas7.1: DiCas7-11 |
| (SEQ ID | KEFVRGQAFARWHRNKKDNTKGRPYITGT | | L1: DiCas7-11 |
| NO: 33) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWT | | |
| | KVGYEIKIQSPLLSNDPIAALFDTANHDAVA | | |
| | YKKRVWENGRIIERHTFKGESIRGVLRTAL | | |
| | GREYGLFELKHEDCPCSLCTIFGNEHEAGKI | | |
| | RFEDLEIEDGVAEKHFDHVAIDRFTGGAVD | | |
| | KKKFDDFSLAGSPGKPIKLKGCFWLRADIF | | |
| | SDEKKDEKKKIINALCDVRDGLYPLGGKGG | | |
| | VGYGWVKELRITDDDKRISRLMNPAFDET | | |
| | DVAVPEKPKTDAEVRIEAEKVYYPHYFVEP | | |
| | HKKVEREEKPCGHQKFHEGRLTGKIECTLK | | |
| | TLSPLIIPDSEVVTENGNGHKSYQFFRLNNEI | | |
| | IIPGSEIRGMISSVYEALTNSCFRVVDDIIPVV | | |
| | ISRSVSPYQIAQKLPSPDLRPCEREILEDSEIG | | |
| | SLDKCSEKRLFRLHEDGLCPSCRLFGTTHY | | |
| | KGRVRFGFAKHEGGEKWLMDDKTKDEYL | | |
| | TLPLLERPRPTWSMPSDEDDVPGRKFYVHH | | |
| | NGWEKVKQDSLDNKTSKTENNRSVEPLDK | | |
| | DNLFKFDVFFENLEPWELGLLLYSLELEEG | | |
| | LAHKFGMAKAFGFGSTKIDADKILLREEAG | | |
| | KFHPCEKADEYLKKGLNKLTSWFGKNEINE | | |
| | HMRNLLLFMTYYQNLPKVKYPDFDGYAK | | |
| | WRCSYVEQDKVEYFQNRWIVAS* | | |
| Cas7-S40 | MTTTMKISIEFLEPFRMTKWQESTRRNKNN | 1255 | Cas7.1: DiCas7-11 |
| (SEQ ID | KEFVRGQAFARWHRNKKDNTKGRPYITGT | | L1: DiCas7-11 |
| NO: 34) | LLRSAVIRSAENLLTLSDGKISEKTCCPGKF | | Cas11: DiCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNANLAEKTAEQI | | Cas7.4: Cas7-1 |
| | ISILDDNKKTEYTRLLADAIRSLRRSSKLVA | | INS: Cas7-1 |
| | GLPKDHDGKDDHYLWDIGKKKKDENSVTI | | CTE: HvsCas7-11 |
| | RQILTTSADTKELKNAGKWREFCEKLGEAL | | |
| | YLKSKDMSGGLKITRRILGDAEFHGKPDRL | | |
| | EKSRSVSIGSVLKETVVCGELVAKTPFFFGA | | |
| | IDEDAKQTDLQVLLTPDNKYRLPRSAVRGI | | |
| | LRRDLQTYFDSPCNAELGGRPCMCKTCRIM | | |
| | RGITVMDARSEYNAPPEIRHRTRINPFTGTV | | |
| | AEGALFNMEVAPEGIVFPFQLRYRGSEDGL | | |
| | PDALKTVLKWWAEGQAFMSGAASTGKGR | | |
| | FRMENAKYETLDLSDENQRNDYLKNWGW | | |
| | RDEKGLEELKKRLNSGLPEPGNYRDPKWH | | |
| | EINVSIEMASPFINGDPIRAAVDKRGTDVVT | | |
| | FVKYKAEGEEAKPVCAYKAESFRGVIRSAV | | |
| | ARIHMEDGVPLTELTHSDCECLLCQIFGSEY | | |
| | EAGKIRFEDLVFESDPEPVTFDHVAIDRFTG | | |
| | GAADKKKFDDSPLPGSPARPLMLKGSFWIR | | |
| | RDVLEDEEYCKALGKALADVNNGLYPLGG | | |
| | KSAIGYGQVKSLGIKGDDKRISRLMNPAFD | | |
| | ETDVAVPEKPKTDAEVRIEAEKVYYPHYFV | | |
| | EPHKKVEREEKPCGHQKFHEGRLTGKIECT | | |
| | LKTLSPLIIPDSEVVTENGNGHKSYQFFRLN | | |
| | NEIIIPGSEIRGMISSVYEALTNSCFRVVDDII | | |
| | PVVISRSVSPYQIAQKLPSPDLRPCEREILED | | |
| | SEIGSLDKCSEKRLFRLHEDGLCPSCRLFGT | | |
| | THYKGRVRFGFAKHEGGEKWLMDDKTKD | | |
| | EYLTLPLLERPRPTWSMPSDEDDVPGRKFY | | |
| | VHHNGWEKVKQDSLDNKTSKTENNRSVEP | | |
| | LDKDNLFKFDVFFENLEPWELGLLLYSLEL | | |
| | EEGLAHKFGMAKAFGFGSTKIDADKILLRE | | |
| | EAGKFHPCEKADEYLKKGLNKLTSWFGKN | | |
| | EINEHMRNLLLFMTYYQNLPKVKYPDFDG | | |
| | YAKWRCSYVEQDKVEYFQNRWIVAS* | | |
| Cas7-S41 | MTTTMKISIEFLEPFRMTKWQESTRRNKNN | 1241 | Cas7.1: DiCas7-11 |
| (SEQ ID | KEFVRGQAFARWHRNKKDNTKGRPYITGT | | L1: DiCas7-11 |
| NO: 35) | LLRSAVIRSAENLLTLSDGKISEKTCCPGKF | | Cas11: HvsCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLYEWIIVGRLIAQTPFHFGDEEKAEGAI | | |
| | LLTPDNRFRLPRTALRGILRRDLKLAGASA | | |
| | CEVEVGRSEPCPCDVCKIMRRVTLLDTVSE | | |
| | DLRDFLPELRKRIRINPQSGTVAEGALFDTE | | |
| | VGPEGLSFPFVLRYKCEKLPDSLTTVLCWW | | |
| | QEGLAFLSGESATGKGRFRLEINGAFVWDL | | |
| | SDENQRNDYLKNWGWRDEKGLEELKKRL | | |
| | NSGLPEPGNYRDPKWDLIKYQLNISSPLLLN | | |
| | DPIRAIALYEGEGKAPDAVFFKKYVFENGKI | | |
| | EEKPCFKAESIRGIFRTAVGRIKNVLTKNHE | | |
| | DCICVLCHLFGNVHETGRLKFEDLKIVSGQ | | |
| | EEKFFDHVAIDRFLGGAKEKYKFDDKPIIGA | | |
| | PDTPIVLEGKIWVKKDINDEAKETLSQAFSD | | |
| | INTGIYYLGANGSIGYGWIEEVKALKDDKRI | | |
| | SRLMNPAFDETDVAVPEKPKTDAEVRIEAE | | |
| | KVYYPHYFVEPHKKVEREEKPCGHQKFHE | | |
| | GRLTGKIECTLKTLSPLIIPDSEVVTENGNG | | |
| | HKSYQFFRLNNEIIIPGSEIRGMISSVYEALT | | |
| | NSCFRVVDDIIPVVISRSVSPYQIAQKLPSPD | | |
| | LRPCEREILEDSEIGSLDKCSEKRLFRLHED | | |
| | GLCPSCRLFGTTHYKGRVRFGFAKHEGGEK | | |
| | WLMDDKTKDEYLTLPLLERPRPTWSMPSD | | |
| | EDDVPGRKFYVHHNGWEKVKQDSLDNKT | | |
| | SKTENNRSVEPLDKDNLFKFDVFFENLEPW | | |
| | ELGLLLYSLELEEGLAHKFGMAKAFGFGST | | |
| | KIDADKILLREEAGKFHPCEKADEYLKKGL | | |
| | NKLTSWFGKNEINEHMRNLLLFMTYYQNL | | |
| | PKVKYPDFDGYAKWRCSYVEQDKVEYFQ | | |
| | NRWIVAS* | | |
| Cas7-S42 | MTTTMKISIEFLEPFRMTKWQESTRRNKNN | 1241 | Cas7.1: DiCas7-11 |
| (SEQ ID | KEFVRGQAFARWHRNKKDNTKGRPYITGT | | L1: DiCas7-11 |
| NO: 36) | LLRSAVIRSAENLLTLSDGKISEKTCCPGKF | | Cas11: HvsCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLYEWIIVGRLIAQTPFHFGDEEKAEGAI | | |
| | LLTPDNRFRLPRTALRGILRRDLKLAGASA | | |
| | CEVEVGRSEPCPCDVCKIMRRVTLLDTVSE | | |
| | DLRDFLPELRKRIRINPQSGTVAEGALFDTE | | |
| | VGPEGLSFPFVLRYKCEKLPDSLTTVLCWW | | |
| | QEGLAFLSGESATGKGRFRLEINGAFVWDL | | |
| | SDENQRNDYLKNWGWRDEKGLEELKKRL | | |
| | NSGLPEPGNYRDPKWTKVGYEIKIQSPLLS | | |
| | NDPIAALFDTANHDAVAYKKRVWENGRIIE | | |
| | RHTFKGESIRGVLRTALGREYGLFELKHED | | |
| | CPCSLCTIFGNEHEAGKIRFEDLEIEDGVAE | | |
| | KHFDHVAIDRFTGGAVDKKKFDDFSLAGSP | | |
| | GKPIKLKGCFWLRADIFSDEKKDEKKKIINA | | |
| | LCDVRDGLYPLGGKGGVGYGWVKELRITD | | |
| | DDKRISRLMNPAFDETDVAVPEKPKTDAEV | | |
| | RIEAEKVYYPHYFVEPHKKVEREEKPCGHQ | | |
| | KFHEGRLTGKIECTLKTLSPLIIPDSEVVTEN | | |
| | GNGHKSYQFFRLNNEIIIPGSEIRGMISSVYE | | |
| | ALTNSCFRVVDDIIPVVISRSVSPYQIAQKLP | | |
| | SPDLRPCEREILEDSEIGSLDKCSEKRLFRLH | | |
| | EDGLCPSCRLFGTTHYKGRVRFGFAKHEGG | | |
| | EKWLMDDKTKDEYLTLPLLERPRPTWSMP | | |
| | SDEDDVPGRKFYVHHNGWEKVKQDSLDN | | |
| | KTSKTENNRSVEPLDKDNLFKFDVFFENLE | | |
| | PWELGLLLYSLELEEGLAHKFGMAKAFGF | | |
| | GSTKIDADKILLREEAGKFHPCEKADEYLK | | |
| | KGLNKLTSWFGKNEINEHMRNLLLFMTYY | | |
| | QNLPKVKYPDFDGYAKWRCSYVEQDKVE | | |
| | YFQNRWIVAS* | | |
| Cas7-S43 | MTTTMKISIEFLEPFRMTKWQESTRRNKNN | 1244 | Cas7.1: DiCas7-11 |
| (SEQ ID | KEFVRGQAFARWHRNKKDNTKGRPYITGT | | L1: DiCas7-11 |
| NO: 37) | LLRSAVIRSAENLLTLSDGKISEKTCCPGKF | | Cas11: HvsCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLYEWIIVGRLIAQTPFHFGDEEKAEGAI | | |
| | LLTPDNRFRLPRTALRGILRRDLKLAGASA | | |
| | CEVEVGRSEPCPCDVCKIMRRVTLLDTVSE | | |
| | DLRDFLPELRKRIRINPQSGTVAEGALFDTE | | |
| | VGPEGLSFPFVLRYKCEKLPDSLTTVLCWW | | |
| | QEGLAFLSGESATGKGRFRLEINGAFVWDL | | |
| | SDENQRNDYLKNWGWRDEKGLEELKKRL | | |
| | NSGLPEPGNYRDPKWHEINVSIEMASPFING | | |
| | DPIRAAVDKRGTDVVTFVKYKAEGEEAKP | | |
| | VCAYKAESFRGVIRSAVARIHMEDGVPLTE | | |
| | LTHSDCECLLCQIFGSEYEAGKIRFEDLVFE | | |
| | SDPEPVTFDHVAIDRFTGGAADKKKFDDSP | | |
| | LPGSPARPLMLKGSFWIRRDVLEDEEYCKA | | |
| | LGKALADVNNGLYPLGGKSAIGYGQVKSL | | |
| | GIKGDDKRISRLMNPAFDETDVAVPEKPKT | | |
| | DAEVRIEAEKVYYPHYFVEPHKKVEREEKP | | |
| | CGHQKFHEGRLTGKIECTLKTLSPLIIPDSEV | | |
| | VTENGNGHKSYQFFRLNNEIIIPGSEIRGMIS | | |
| | SVYEALTNSCFRVVDDIIPVVISRSVSPYQIA | | |
| | QKLPSPDLRPCEREILEDSEIGSLDKCSEKRL | | |
| | FRLHEDGLCPSCRLFGTTHYKGRVRFGFAK | | |
| | HEGGEKWLMDDKTKDEYLTLPLLERPRPT | | |
| | WSMPSDEDDVPGRKFYVHHNGWEKVKQD | | |
| | SLDNKTSKTENNRSVEPLDKDNLFKFDVFF | | |
| | ENLEPWELGLLLYSLELEEGLAHKFGMAK | | |
| | AFGFGSTKIDADKILLREEAGKFHPCEKADE | | |
| | YLKKGLNKLTSWFGKNEINEHMRNLLLFM | | |
| | TYYQNLPKVKYPDFDGYAKWRCSYVEQD | | |
| | KVEYFQNRWIVAS* | | |
| Cas7-S44 | MTTTMKISIEFLEPFRMTKWQESTRRNKNN | 1244 | Cas7.1: DiCas7-11 |
| (SEQ ID | KEFVRGQAFARWHRNKKDNTKGRPYITGT | | L1: DiCas7-11 |
| NO: 38) | DTEDKDRLLQLRQRSTLRWTDKNPCPDNA | | Cas11: HvsCas7-11 |
| | LLRSAVIRSAENLLTLSDGKISEKTCCPGKF | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLKETVVCGELVAKTPFFFGAIDEDAK | | |
| | QTDLQVLLTPDNKYRLPRSAVRGILRRDLQ | | |
| | TYFDSPCNAELGGRPCMCKTCRIMRGITVM | | |
| | DARSEYNAPPEIRHRTRINPFTGTVAEGALF | | |
| | NMEVAPEGIVFPFQLRYRGSEDGLPDALKT | | |
| | VLKWWAEGQAFMSGAASTGKGRFRMENA | | |
| | KYETLDLSDENQRNDYLKNWGWRDEKGL | | |
| | EELKKRLNSGLPEPGNYRDPKWDLIKYQLN | | |
| | ISSPLLLNDPIRAIALYEGEGKAPDAVFFKK | | |
| | YVFENGKIEEKPCFKAESIRGIFRTAVGRIK | | |
| | NVLTKNHEDCICVLCHLFGNVHETGRLKFE | | |
| | DLKIVSGQEEKFFDHVAIDRFLGGAKEKYK | | |
| | FDDKPIIGAPDTPIVLEGKIWVKKDINDEAK | | |
| | ETLSQAFSDINTGIYYLGANGSIGYGWIEEV | | |
| | KALKDDKRISRLMNPAFDETDVAVPEKPKT | | |
| | DAEVRIEAEKVYYPHYFVEPHKKVEREEKP | | |
| | CGHQKFHEGRLTGKIECTLKTLSPLIIPDSEV | | |
| | VTENGNGHKSYQFFRLNNEIIIPGSEIRGMIS | | |
| | SVYEALTNSCFRVVDDIIPVVISRSVSPYQIA | | |
| | QKLPSPDLRPCEREILEDSEIGSLDKCSEKRL | | |
| | FRLHEDGLCPSCRLFGTTHYKGRVRFGFAK | | |
| | HEGGEKWLMDDKTKDEYLTLPLLERPRPT | | |
| | WSMPSDEDDVPGRKFYVHHNGWEKVKQD | | |
| | SLDNKTSKTENNRSVEPLDKDNLFKFDVFF | | |
| | ENLEPWELGLLLYSLELEEGLAHKFGMAK | | |
| | AFGFGSTKIDADKILLREEAGKFHPCEKADE | | |
| | YLKKGLNKLTSWFGKNEINEHMRNLLLFM | | |
| | TYYQNLPKVKYPDFDGYAKWRCSYVEQD | | |
| | KVEYFQNRWIVAS* | | |
| Cas7-S45 | MTTTMKISIEFLEPFRMTKWQESTRRNKNN | 1244 | Cas7.1: DiCas7-11 |
| (SEQ ID | KEFVRGQAFARWHRNKKDNTKGRPYITGT | | L1: DiCas7-11 |
| NO: 39) | LLRSAVIRSAENLLTLSDGKISEKTCCPGKF | | Cas11: HvsCas7-11 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLKETVVCGELVAKTPFFFGAIDEDAK | | |
| | QTDLQVLLTPDNKYRLPRSAVRGILRRDLQ | | |
| | TYFDSPCNAELGGRPCMCKTCRIMRGITVM | | |
| | DARSEYNAPPEIRHRTRINPFTGTVAEGALF | | |
| | NMEVAPEGIVFPFQLRYRGSEDGLPDALKT | | |
| | VLKWWAEGQAFMSGAASTGKGRFRMENA | | |
| | KYETLDLSDENQRNDYLKNWGWRDEKGL | | |
| | EELKKRLNSGLPEPGNYRDPKWTKVGYEIK | | |
| | IQSPLLSNDPIAALFDTANHDAVAYKKRVW | | |
| | ENGRIIERHTFKGESIRGVLRTALGREYGLF | | |
| | ELKHEDCPCSLCTIFGNEHEAGKIRFEDLEIE | | |
| | DGVAEKHFDHVAIDRFTGGAVDKKKFDDF | | |
| | SLAGSPGKPIKLKGCFWLRADIFSDEKKDE | | |
| | KKKIINALCDVRDGLYPLGGKGGVGYGWV | | |
| | KELRITDDDKRISRLMNPAFDETDVAVPEK | | |
| | PKTDAEVRIEAEKVYYPHYFVEPHKKVERE | | |
| | EKPCGHQKFHEGRLTGKIECTLKTLSPLIIPD | | |
| | SEVVTENGNGHKSYQFFRLNNEIIIPGSEIRG | | |
| | MISSVYEALTNSCFRVVDDIIPVVISRSVSPY | | |
| | QIAQKLPSPDLRPCEREILEDSEIGSLDKCSE | | |
| | KRLFRLHEDGLCPSCRLFGTTHYKGRVRFG | | |
| | FAKHEGGEKWLMDDKTKDEYLTLPLLERP | | |
| | RPTWSMPSDEDDVPGRKFYVHHNGWEKV | | |
| | KQDSLDNKTSKTENNRSVEPLDKDNLFKFD | | |
| | VFFENLEPWELGLLLYSLELEEGLAHKFGM | | |
| | AKAFGFGSTKIDADKILLREEAGKFHPCEK | | |
| | ADEYLKKGLNKLTSWFGKNEINEHMRNLL | | |
| | LFMTYYQNLPKVKYPDFDGYAKWRCSYV | | |
| | EQDKVEYFQNRWIVAS* | | |
| Cas7-S46 | MTTTMKISIEFLEPFRMTKWQESTRRNKNN | 1247 | Cas7.1: DiCas7-11 |
| (SEQ ID | KEFVRGQAFARWHRNKKDNTKGRPYITGT | | L1: DiCas7-11 |
| NO: 40) | DTEDKDRLLQLRQRSTLRWTDKNPCPDNA | | Cas11: HvsCas7-11 |
| | LLRSAVIRSAENLLTLSDGKISEKTCCPGKF | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAQLSKQAKED | | Cas7.4: Cas7-1 |
| | IEKIFQVVKDAQKLRLLSDCFRELTRMANK | | INS: Cas7-1 |
| | DELALPLGPEDDGHYLWDKIKVEGKTLRIF | | CTE: HvsCas7-11 |
| | LRNCFSQYKDNWLCFCDEASKKGYQKYRE | | |
| | MSGGLKITRRILGDAEFHGKPDRLEKSRSVS | | |
| | IGSVLKETVVCGELVAKTPFFFGAIDEDAK | | |
| | QTDLQVLLTPDNKYRLPRSAVRGILRRDLQ | | |
| | TYFDSPCNAELGGRPCMCKTCRIMRGITVM | | |
| | DARSEYNAPPEIRHRTRINPFTGTVAEGALF | | |
| | NMEVAPEGIVFPFQLRYRGSEDGLPDALKT | | |
| | VLKWWAEGQAFMSGAASTGKGRFRMENA | | |
| | KYETLDLSDENQRNDYLKNWGWRDEKGL | | |
| | EELKKRLNSGLPEPGNYRDPKWHEINVSIE | | |
| | MASPFINGDPIRAAVDKRGTDVVTFVKYKA | | |
| | EGEEAKPVCAYKAESFRGVIRSAVARIHME | | |
| | DGVPLTELTHSDCECLLCQIFGSEYEAGKIR | | |
| | FEDLVFESDPEPVTFDHVAIDRFTGGAADK | | |
| | KKFDDSPLPGSPARPLMLKGSFWIRRDVLE | | |
| | DEEYCKALGKALADVNNGLYPLGGKSAIG | | |
| | YGQVKSLGIKGDDKRISRLMNPAFDETDVA | | |
| | VPEKPKTDAEVRIEAEKVYYPHYFVEPHKK | | |
| | VEREEKPCGHQKFHEGRLTGKIECTLKTLSP | | |
| | LIIPDSEVVTENGNGHKSYQFFRLNNEIIIPG | | |
| | SEIRGMISSVYEALTNSCFRVVDDIIPVVISR | | |
| | SVSPYQIAQKLPSPDLRPCEREILEDSEIGSL | | |
| | DKCSEKRLFRLHEDGLCPSCRLFGTTHYKG | | |
| | RVRFGFAKHEGGEKWLMDDKTKDEYLTLP | | |
| | LLERPRPTWSMPSDEDDVPGRKFYVHHNG | | |
| | WEKVKQDSLDNKTSKTENNRSVEPLDKDN | | |
| | LFKFDVFFENLEPWELGLLLYSLELEEGLA | | |
| | HKFGMAKAFGFGSTKIDADKILLREEAGKF | | |
| | HPCEKADEYLKKGLNKLTSWFGKNEINEH | | |
| | MRNLLLFMTYYQNLPKVKYPDFDGYAKW | | |
| | RCSYVEQDKVEYFQNRWIVAS* | | |
| Cas7-S47 | MTTTMKISIEFLEPFRMTKWQESTRRNKNN | 1286 | Cas7.1: DiCas7-11 |
| (SEQ ID | KEFVRGQAFARWHRNKKDNTKGRPYITGT | | L1: DiCas7-11 |
| NO: 41) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLYEWIIVGRLIAQT | | |
| | PFHFGDEEKAEGAILLTPDNRFRLPRTALRG | | |
| | ILRRDLKLAGASACEVEVGRSEPCPCDVCKI | | |
| | MRRVTLLDTVSEDLRDFLPELRKRIRINPQS | | |
| | GTVAEGALFDTEVGPEGLSFPFVLRYKCEK | | |
| | LPDSLTTVLCWWQEGLAFLSGESATGKGRF | | |
| | RLEINGAFVWDLSDENQRNDYLKNWGWR | | |
| | DEKGLEELKKRLNSGLPEPGNYRDPKWDLI | | |
| | KYQLNISSPLLLNDPIRAIALYEGEGKAPDA | | |
| | VFFKKYVFENGKIEEKPCFKAESIRGIFRTA | | |
| | VGRIKNVLTKNHEDCICVLCHLFGNVHETG | | |
| | RLKFEDLKIVSGQEEKFFDHVAIDRFLGGA | | |
| | KEKYKFDDKPIIGAPDTPIVLEGKIWVKKDI | | |
| | NDEAKETLSQAFSDINTGIYYLGANGSIGYG | | |
| | WIEEVKALKDDKRISRLMNPAFDETDVAVP | | |
| | EKPKTDAEVRIEAEKVYYPHYFVEPHKKVE | | |
| | REEKPCGHQKFHEGRLTGKIECTLKTLSPLII | | |
| | PDSEVVTENGNGHKSYQFFRLNNEIIIPGSEI | | |
| | RGMISSVYEALTNSCFRVVDDIIPVVISRSVS | | |
| | PYQIAQKLPSPDLRPCEREILEDSEIGSLDKC | | |
| | SEKRLFRLHEDGLCPSCRLFGTTHYKGRVR | | |
| | FGFAKHEGGEKWLMDDKTKDEYLTLPLLE | | |
| | RPRPTWSMPSDEDDVPGRKFYVHHNGWEK | | |
| | VKQDSLDNKTSKTENNRSVEPLDKDNLFKF | | |
| | DVFFENLEPWELGLLLYSLELEEGLAHKFG | | |
| | MAKAFGFGSTKIDADKILLREEAGKFHPCE | | |
| | KADEYLKKGLNKLTSWFGKNEINEHMRNL | | |
| | LLFMTYYQNLPKVKYPDFDGYAKWRCSY | | |
| | VEQDKVEYFQNRWIVAS* | | |
| Cas7-S48 | MTTTMKISIEFLEPFRMTKWQESTRRNKNN | 1286 | Cas7.1: DiCas7-11 |
| (SEQ ID | KEFVRGQAFARWHRNKKDNTKGRPYITGT | | L1: DiCas7-11 |
| NO: 42) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLYEWIIVGRLIAQT | | |
| | PFHFGDEEKAEGAILLTPDNRFRLPRTALRG | | |
| | ILRRDLKLAGASACEVEVGRSEPCPCDVCKI | | |
| | MRRVTLLDTVSEDLRDFLPELRKRIRINPQS | | |
| | GTVAEGALFDTEVGPEGLSFPFVLRYKCEK | | |
| | LPDSLTTVLCWWQEGLAFLSGESATGKGRF | | |
| | RLEINGAFVWDLSDENQRNDYLKNWGWR | | |
| | DEKGLEELKKRLNSGLPEPGNYRDPKWTK | | |
| | VGYEIKIQSPLLSNDPIAALFDTANHDAVAY | | |
| | KKRVWENGRIIERHTFKGESIRGVLRTALG | | |
| | REYGLFELKHEDCPCSLCTIFGNEHEAGKIR | | |
| | FEDLEIEDGVAEKHFDHVAIDRFTGGAVDK | | |
| | KKFDDFSLAGSPGKPIKLKGCFWLRADIFS | | |
| | DEKKDEKKKIINALCDVRDGLYPLGGKGG | | |
| | VGYGWVKELRITDDDKRISRLMNPAFDET | | |
| | DVAVPEKPKTDAEVRIEAEKVYYPHYFVEP | | |
| | HKKVEREEKPCGHQKFHEGRLTGKIECTLK | | |
| | TLSPLIIPDSEVVTENGNGHKSYQFFRLNNEI | | |
| | IIPGSEIRGMISSVYEALTNSCFRVVDDIIPVV | | |
| | ISRSVSPYQIAQKLPSPDLRPCEREILEDSEIG | | |
| | SLDKCSEKRLFRLHEDGLCPSCRLFGTTHY | | |
| | KGRVRFGFAKHEGGEKWLMDDKTKDEYL | | |
| | TLPLLERPRPTWSMPSDEDDVPGRKFYVHH | | |
| | NGWEKVKQDSLDNKTSKTENNRSVEPLDK | | |
| | DNLFKFDVFFENLEPWELGLLLYSLELEEG | | |
| | LAHKFGMAKAFGFGSTKIDADKILLREEAG | | |
| | KFHPCEKADEYLKKGLNKLTSWFGKNEINE | | |
| | HMRNLLLFMTYYQNLPKVKYPDFDGYAK | | |
| | WRCSYVEQDKVEYFQNRWIVAS* | | |
| Cas7-S49 | MTTTMKISIEFLEPFRMTKWQESTRRNKNN | 1289 | Cas7.1: DiCas7-11 |
| (SEQ ID | KEFVRGQAFARWHRNKKDNTKGRPYITGT | | L1: DiCas7-11 |
| NO: 43) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: HvsCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLYEWIIVGRLIAQT | | |
| | PFHFGDEEKAEGAILLTPDNRFRLPRTALRG | | |
| | ILRRDLKLAGASACEVEVGRSEPCPCDVCKI | | |
| | MRRVTLLDTVSEDLRDFLPELRKRIRINPQS | | |
| | GTVAEGALFDTEVGPEGLSFPFVLRYKCEK | | |
| | LPDSLTTVLCWWQEGLAFLSGESATGKGRF | | |
| | RLEINGAFVWDLSDENQRNDYLKNWGWR | | |
| | DEKGLEELKKRLNSGLPEPGNYRDPKWHEI | | |
| | NVSIEMASPFINGDPIRAAVDKRGTDVVTF | | |
| | VKYKAEGEEAKPVCAYKAESFRGVIRSAV | | |
| | ARIHMEDGVPLTELTHSDCECLLCQIFGSEY | | |
| | EAGKIRFEDLVFESDPEPVTFDHVAIDRFTG | | |
| | GAADKKKFDDSPLPGSPARPLMLKGSFWIR | | |
| | RDVLEDEEYCKALGKALADVNNGLYPLGG | | |
| | KSAIGYGQVKSLGIKGDDKRISRLMNPAFD | | |
| | ETDVAVPEKPKTDAEVRIEAEKVYYPHYFV | | |
| | EPHKKVEREEKPCGHQKFHEGRLTGKIECT | | |
| | LKTLSPLIIPDSEVVTENGNGHKSYQFFRLN | | |
| | NEIIIPGSEIRGMISSVYEALTNSCFRVVDDII | | |
| | PVVISRSVSPYQIAQKLPSPDLRPCEREILED | | |
| | SEIGSLDKCSEKRLFRLHEDGLCPSCRLFGT | | |
| | THYKGRVRFGFAKHEGGEKWLMDDKTKD | | |
| | EYLTLPLLERPRPTWSMPSDEDDVPGRKFY | | |
| | VHHNGWEKVKQDSLDNKTSKTENNRSVEP | | |
| | LDKDNLFKFDVFFENLEPWELGLLLYSLEL | | |
| | EEGLAHKFGMAKAFGFGSTKIDADKILLRE | | |
| | EAGKFHPCEKADEYLKKGLNKLTSWFGKN | | |
| | EINEHMRNLLLFMTYYQNLPKVKYPDFDG | | |
| | YAKWRCSYVEQDKVEYFQNRWIVAS* | | |
| Cas7-S50 | MTTTMKISIEFLEPFRMTKWQESTRRNKNN | 1289 | Cas7.1: DiCas7-11 |
| (SEQ ID | KEFVRGQAFARWHRNKKDNTKGRPYITGT | | L1: DiCas7-11 |
| NO: 44) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: HvsCas7-11 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLKETVVCGELVAK | | |
| | TPFFFGAIDEDAKQTDLQVLLTPDNKYRLP | | |
| | RSAVRGILRRDLQTYFDSPCNAELGGRPCM | | |
| | CKTCRIMRGITVMDARSEYNAPPEIRHRTRI | | |
| | NPFTGTVAEGALFNMEVAPEGIVFPFQLRY | | |
| | RGSEDGLPDALKTVLKWWAEGQAFMSGA | | |
| | ASTGKGRFRMENAKYETLDLSDENQRNDY | | |
| | LKNWGWRDEKGLEELKKRLNSGLPEPGNY | | |
| | RDPKWDLIKYQLNISSPLLLNDPIRAIALYE | | |
| | GEGKAPDAVFFKKYVFENGKIEEKPCFKAE | | |
| | SIRGIFRTAVGRIKNVLTKNHEDCICVLCHL | | |
| | FGNVHETGRLKFEDLKIVSGQEEKFFDHVA | | |
| | IDRFLGGAKEKYKFDDKPIIGAPDTPIVLEG | | |
| | KIWVKKDINDEAKETLSQAFSDINTGIYYLG | | |
| | ANGSIGYGWIEEVKALKDDKRISRLMNPAF | | |
| | DETDVAVPEKPKTDAEVRIEAEKVYYPHYF | | |
| | VEPHKKVEREEKPCGHQKFHEGRLTGKIEC | | |
| | TLKTLSPLIIPDSEVVTENGNGHKSYQFFRL | | |
| | NNEIIIPGSEIRGMISSVYEALTNSCFRVVDD | | |
| | IIPVVISRSVSPYQIAQKLPSPDLRPCEREILE | | |
| | DSEIGSLDKCSEKRLFRLHEDGLCPSCRLFG | | |
| | TTHYKGRVRFGFAKHEGGEKWLMDDKTK | | |
| | DEYLTLPLLERPRPTWSMPSDEDDVPGRKF | | |
| | YVHHNGWEKVKQDSLDNKTSKTENNRSV | | |
| | EPLDKDNLFKFDVFFENLEPWELGLLLYSL | | |
| | ELEEGLAHKFGMAKAFGFGSTKIDADKILL | | |
| | REEAGKFHPCEKADEYLKKGLNKLTSWFG | | |
| | KNEINEHMRNLLLFMTYYQNLPKVKYPDF | | |
| | DGYAKWRCSYVEQDKVEYFQNRWIVAS* | | |
| Cas7-S51 | MTTTMKISIEFLEPFRMTKWQESTRRNKNN | 1289 | Cas7.1: DiCas7-11 |
| (SEQ ID | KEFVRGQAFARWHRNKKDNTKGRPYITGT | | L1: DiCas7-11 |
| NO: 45) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: Cas7-1 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLKETVVCGELVAK | | |
| | TPFFFGAIDEDAKQTDLQVLLTPDNKYRLP | | |
| | RSAVRGILRRDLQTYFDSPCNAELGGRPCM | | |
| | CKTCRIMRGITVMDARSEYNAPPEIRHRTRI | | |
| | NPFTGTVAEGALFNMEVAPEGIVFPFQLRY | | |
| | RGSEDGLPDALKTVLKWWAEGQAFMSGA | | |
| | ASTGKGRFRMENAKYETLDLSDENQRNDY | | |
| | LKNWGWRDEKGLEELKKRLNSGLPEPGNY | | |
| | RDPKWTKVGYEIKIQSPLLSNDPIAALFDTA | | |
| | NHDAVAYKKRVWENGRIIERHTFKGESIRG | | |
| | VLRTALGREYGLFELKHEDCPCSLCTIFGNE | | |
| | HEAGKIRFEDLEIEDGVAEKHFDHVAIDRFT | | |
| | GGAVDKKKFDDFSLAGSPGKPIKLKGCFW | | |
| | LRADIFSDEKKDEKKKIINALCDVRDGLYPL | | |
| | GGKGGVGYGWVKELRITDDDKRISRLMNP | | |
| | AFDETDVAVPEKPKTDAEVRIEAEKVYYPH | | |
| | YFVEPHKKVEREEKPCGHQKFHEGRLTGKI | | |
| | ECTLKTLSPLIIPDSEVVTENGNGHKSYQFF | | |
| | RLNNEIIIPGSEIRGMISSVYEALTNSCFRVV | | |
| | DDIIPVVISRSVSPYQIAQKLPSPDLRPCEREI | | |
| | LEDSEIGSLDKCSEKRLFRLHEDGLCPSCRL | | |
| | FGTTHYKGRVRFGFAKHEGGEKWLMDDK | | |
| | TKDEYLTLPLLERPRPTWSMPSDEDDVPGR | | |
| | KFYVHHNGWEKVKQDSLDNKTSKTENNRS | | |
| | VEPLDKDNLFKFDVFFENLEPWELGLLLYS | | |
| | LELEEGLAHKFGMAKAFGFGSTKIDADKIL | | |
| | LREEAGKFHPCEKADEYLKKGLNKLTSWF | | |
| | GKNEINEHMRNLLLFMTYYQNLPKVKYPD | | |
| | FDGYAKWRCSYVEQDKVEYFQNRWIVAS* | | |
| Cas7-S52 | MTTTMKISIEFLEPFRMTKWQESTRRNKNN | 1292 | Cas7.1: DiCas7-11 |
| (SEQ ID | KEFVRGQAFARWHRNKKDNTKGRPYITGT | | L1: DiCas7-11 |
| NO: 46) | LLRSAVIRSAENLLTLSDGKISEKTCCPGKF | | Cas1: Cas7-1 |
| | DTEDKDRLLQLRQRSTLRWTDKNPCPDNA | | L2: DiCas7-11 |
| | ETYCPFCELLGRSGNDGKKAEKKDWRFRIH | | Cas7.2: DiCas7-11 |
| | FGNLSLPGKPDFDGPKAIGSQRVLNRVDFK | | L3: DiCas7-11 |
| | SGKAHDFFKAYEVDHTRFPRFEGEITIDNK | | Cas7.3: DiCas7-11 |
| | VSAEARKLLCDSLKFTDRLCGALCVIRFDE | | L4: DiCas7-11 |
| | YTPAADSGKQTENVQAEPNAAVKKAAGDL | | Cas7.4: Cas7-1 |
| | VNSALNYGYGILYGRSINAIIQAGLNPMAG | | INS: Cas7-1 |
| | FLHSYQPTKPVLVYDLVEEFRPLAVDRGIFT | | CTE: HvsCas7-11 |
| | LLNRGQKIELGRDSLLSAKSRKKIAKSVIGC | | |
| | LSSEVCFNGHRLSLEDVIKEQALNIKKHLY | | |
| | NNAKYRPFLGRWMSGGLKITRRILGDAEFH | | |
| | GKPDRLEKSRSVSIGSVLKETVVCGELVAK | | |
| | TPFFFGAIDEDAKQTDLQVLLTPDNKYRLP | | |
| | RSAVRGILRRDLQTYFDSPCNAELGGRPCM | | |
| | CKTCRIMRGITVMDARSEYNAPPEIRHRTRI | | |
| | NPFTGTVAEGALFNMEVAPEGIVFPFQLRY | | |
| | RGSEDGLPDALKTVLKWWAEGQAFMSGA | | |
| | ASTGKGRFRMENAKYETLDLSDENQRNDY | | |
| | LKNWGWRDEKGLEELKKRLNSGLPEPGNY | | |
| | RDPKWHEINVSIEMASPFINGDPIRAAVDKR | | |
| | GTDVVTFVKYKAEGEEAKPVCAYKAESFR | | |
| | GVIRSAVARIHMEDGVPLTELTHSDCECLL | | |
| | CQIFGSEYEAGKIRFEDLVFESDPEPVTFDH | | |
| | VAIDRFTGGAADKKKFDDSPLPGSPARPLM | | |
| | LKGSFWIRRDVLEDEEYCKALGKALADVN | | |
| | NGLYPLGGKSAIGYGQVKSLGIKGDDKRIS | | |
| | RLMNPAFDETDVAVPEKPKTDAEVRIEAEK | | |
| | VYYPHYFVEPHKKVEREEKPCGHQKFHEG | | |
| | RLTGKIECTLKTLSPLIIPDSEVVTENGNGH | | |
| | KSYQFFRLNNEIIIPGSEIRGMISSVYEALTN | | |
| | SCFRVVDDIIPVVISRSVSPYQIAQKLPSPDL | | |
| | RPCEREILEDSEIGSLDKCSEKRLFRLHEDG | | |
| | LCPSCRLFGTTHYKGRVRFGFAKHEGGEK | | |
| | WLMDDKTKDEYLTLPLLERPRPTWSMPSD | | |
| | EDDVPGRKFYVHHNGWEKVKQDSLDNKT | | |
| | SKTENNRSVEPLDKDNLFKFDVFFENLEPW | | |
| | ELGLLLYSLELEEGLAHKFGMAKAFGFGST | | |
| | KIDADKILLREEAGKFHPCEKADEYLKKGL | | |
| | NKLTSWFGKNEINEHMRNLLLFMTYYQNL | | |
| | PKVKYPDFDGYAKWRCSYVEQDKVEYFQ | | |
| | NRWIVAS* | | |
| Cas7-S54 | MTTTMKISIEFLEPFRMTKWQESTRRNKNN | 1188 | Cas7.1: DiCas7-11 |
| (SEQ ID | KEFVRGQAFARWHRNKKDNTKGRPYITGT | | Linker 1: DiCas7- |
| NO: 47) | LLRSAVIRSAENLLTLSDGKISEKTCCPGKF | | 11 |
| | DTEDKDRLLQLRQRSTLRWTDKNPCPDNA | | Cas11: HsmCas7-11 |
| | ETYCPFCELLGRSGNDGKKAEKKDWRFRIH | | Linker 2: HsmCas7- |
| | FGNLSLPGKPDFDGPKAIGSQRVLNRVDFK | | 11 |
| | SGKAHDFFKAYEVDHTRFPRFEGEITIDNK | | Cas7.2:HvsCas7-11 |
| | VSAEARKLLCDSLKFTDRLCGALCVIRFDE | | Linker 3: SstCas7- |
| | YTPAADSGKQTENVQAEPNAEEIKKLAQEI | | 11 |
| | RDILKGEDALDKMRVLADTVLQMRTKGPE | | Cas7.3: OmCas7-11 |
| | IVNELPRGIEKKGGHWLWDKLRLRKKFKEI | | Linker 4: DiCas7- |
| | ANNYKDSWQELCEKLGNELYISYKELTGGI | | 11 |
| | AVKKRIIGETEYRKIPEQEISFLPSKAGYSYE | | Cas7.4: CsbCas7-11 |
| | WIIVGRLIAQTPFHFGDEEKAEGAILLTPDN | | INS: Cas7-1 |
| | RFRLPRTALRGILRRDLKLAGASACEVEVG | | CTE: CbfCas7-11 |
| | RSEPCPCDVCKIMRRVTLLDTVSEDLRDFL | | |
| | PELRKRIRINPQSGTVAEGALFDTEVGPEGL | | |
| | SFPFVLRYKCEKLPDSLTTVLCWWQEGLAF | | |
| | LSGESATGKGRFRLEINGAFVWDLSDKDKR | | |
| | KDYLDNYGWRNRIEALSLEKMPLDRMND | | |
| | YAEPLWKKHAWKLSFQVPLLTADPIMAQT | | |
| | RHEEDSVYFQKRIFTSDGRVVLVPALRGEG | | |
| | LRGLLRTAVSRAYGISLINDEHEDCDCPLCK | | |
| | IFGNEHHAGMLRFDDMVPVGTWNDKKIDH | | |
| | VSCSRFDASVVNKFDDRSLVGSPDSPLHFE | | |
| | GTFWLHRDFQNDVEIKTALQDFADGLYSIG | | |
| | GKGGIGYGWLFDMEIPRDDKRISRLMNPAF | | |
| | DETDVAVPEKPKTDAEVRIEAEKVYYPHYF | | |
| | VEPHKKVEREEKPCGHQKFHEGRLSGKINC | | |
| | KLETLTPLIIPDTSDENGLKLQGNKPGHKNY | | |
| | KFFNINGELMIPGSELRGMLRTHFEALTKSC | | |
| | FAIFGEDSTLSWRRSVSPYRKCASKTLGGK | | |
| | LDKALHPCTGLSDGLCPGCHLFGTTDYKGR | | |
| | VKFGFAKYENGPEWLITRGNNPERSLTLGV | | |
| | LESPRPAFSIPDDESEIPGRKFYLHHNGWRII | | |
| | RQKQLEIRETVQPERNVTTEVMDKGNVFSF | | |
| | DVRFENLREWELGLLLQSLDPGKNIAHKLG | | |
| | KGKPYGFGSVKIKIDSLHTFVVNVKDGCVL | | |
| | WEPEEDKIQHYIAKGLGKLTTWFGKEWDR | | |
| | LEHIQGLRSLQRLLPL | | |

TABLE 3
List of crRNAs used. The table indicates the target of the crRNA.
Further, the sequences in Table 3 include a direct repeat listed in Table 5.
NT indicated non-target crRNA.

| SEQ | | | |
|---|---|---|---|
| ID | | | |
| NO: | crRNA | Spacer Target | RNA sequence (5'->3') |
| 48 | Di-crRNA-EGFP | EGFP | gguuggaaagccgguuuucuuugaugucacggaacuucaccuugaug |
| | | | ccguucuucugcuugucg |
| 49 | Di-crRNA-NT | NT | gguuggaaagcegguuuucuuugaugucacggaaccuaccugguage |
| | | | ccuuguauuugaucagge |
| 50 | Rfx-crRNA-EGFP | EGFP | caaguaaaccccuaccaacuggucgggguuugaaacuucaccuugau |
| | | | gccguucuucu |
| 51 | Di-crRNA-EGFP-22 | EGFP | gguuggaaagccgguuuucuuugaugucacggaacuucaccuugaug |
| | | | ccguucuucu |
| 52 | Di-crRNA-NT-22 | NT | gguuggaaagccgguuuucuuugaugucacggaaccuaccugguagc |
| | | | ccuuguauuu |
| 53 | Di-crRNA-CoV2-1 | SARS-COV-2 | gguuggaaagcegguuuuuuugaugucacggaacaauuucuguggua |
| | | | acacuaaua |
| 54 | Di-crRNA-CoV2-2 | SARS-COV-2 | gguuggaaagcegguuuucuuugaugucacggaacagaauuucugug |
| | | | guaacacuaa |
| 55 | Di-crRNA-CoV2-3 | SARS-COV-2 | gguuggaaagcegguuuucuuugaugucacggaacguagaauuucug |
| | | | ugguaacacu |
| 56 | Di-critNA-CoV2-4 | SARS-COV-2 | gguuggaaagccgguuuucuuugaugucacggaacgguagaauuucu |
| | | | gugguaacac |
| 57 | Di-crRNA-CoV2-5 | SARS-COV-2 | gguuggaaagcegguuuucuuugaugucacggaaccugguagaauuu |
| | | | cugugguaac |
| 58 | Di-crRNA-CoV2-6 | SARS-COV-2 | gguuggaaagcegguuuucuuugaugucacggaaccacugguagaau |
| | | | uucuguggua |
| 59 | Di-crRNA-CoV2-2-30 | SARS-COV-2 | gguuggaaagccgguuuucuuugaugucacggaaccugguagaauuu |
| | | | cugugguaacacuaauag |
| 60 | Di-crRNA-CoV2-2-28 | SARS-COV-2 | gguuggaaagcegguuuucuuugaugucacggaaccugguagaauuu |
| | | | cugugguaacacuaau |
| 61 | Di-crRNA-CoV2-2-26 | SARS-COV-2 | gguuggaaagcegguuuucuuugaugucacggaaccugguagaauuu |
| | | | cugugguaacacua |
| 62 | Di-crRNA-CoV2-2-24 | SARS-COV-2 | gguuggaaagcegguuuucuuugaugucacggaaccugguagaauuu |
| | | | cugugguaacac |
| 63 | Di-crRNA-CoV2-2-20 | SARS-COV-2 | gguuggaaagcegguuuucuuugaugucacggaaccugguagaauuu |
| | | | cuguggua |
| 64 | Di-crRNA-array | SARS-COV-2 | gguuggaaagccgguuuucuuugaugucacggaaccugguagaauuu |
| | | | cugugguaacgguuggaaagcegguuuucuuugaugucacggaac |
| 65 | Di-crRNA-mDR-array | SARS-COV-2 | uugaugucacggaaccugguagaauuucugugguaacuugaugucac |
| | | | ggaac |
| 66 | Hvs-cRNA-EGFP | EGFP | gaaacaccguuugaaggaggauaauaagguugauguuucaccuugau |
| | | | gccguucuucugcuugucg |
| 67 | Hvs-crRNA-NT | NT | gaaacaccguuugaaggaggauaauaagguugaugucuaccugguag |
| | | | cccuuguauuugaucagge |
| 68 | Rfx-crRNA-CoV2 | SARS-COV-2 | caaguaaaccccuaccaacugguegggguuugaaacguagaauuucu |
| | | | gugguaacacuaauaguaa |
| 69 | Di-crRNA-mCherry | mCherry | gguuggaaagcegguuuucuuugaugucacggaaccaccuugaageg |
| | | | eaugaacucc |
| 70 | Di-crRNA-KRAS | KRAS | gguuggaaagcegguuuucuuugaugucacggaacgguuaaaaauuu |
| | | | acagauugugcu |
| 71 | 71-DRX-F-CoV2-5 | SARS-COV-2 | uuguuucaguuccugeeggcaggaauguccuuucagaccugguagaa |
| | | | uuucugugguaac |
| 72 | 71-DRX-R-COV2-5 | SARS-COV-2 | cugguagaauuucugugguaacuuguuucaguuccugccggcaggaa |
| | | | uguccuuucagac |
| 73 | 71-DRY-F-CoV2-5 | SARS-COV-2 | gucugaaaggacauuccugceggcaggaacugaaacaacugguagaa |
| | | | uuucugugguaac |
| 74 | 71-DRY-F-CoV2-5 | SARS-COV-2 | cugguagaauuucugugguaacgucugaaaggacauuccugceggca |
| | | | ggaacugaaacaa |

TABLE 4
Sequences of plasmids used with respective Addgene IDs.

| Construct | | | |
|---|---|---|---|
| No: | Construct | Application | Addgene ID |
| 1 | CMV-SynCas.v10-bGH-AmpR | Mammalian | 208589 |
| | | expression | |
| 2 | CMV-SynCas.v14A-bGH-AmpR | Mammalian | 208590 |
| | | expression | |
| 3 | CMV-SynCas.v35-bGH-AmpR | Mammalian | 208591 |
| | | expression | |
| 4 | CMV-SynCas.v39-bGH-AmpR | Mammalian | 208592 |
| | | expression | |
| 5 | CMV-SynCas.v41-bGH-AmpR | Mammalian | 208593 |
| | | expression | |
| 6 | CMV-SynCas.v47-bGH-AmpR | Mammalian | 209003 |
| | | expression | |
| 7 | T7-RBS-6xHis-SUMO-SynCas.v10-term-KanR | Protein | 208594 |
| | | purification | |
| 8 | T7-RBS-6xHis-SUMO-dSynCas.v10-term-KanR | Protein | 208595 |
| | | purification | |
| 9 | T7-RBS-6xHis-SUMO-SynCas.v12-term-KanR | Protein | 208596 |
| | | purification | |
| 10 | T7-RBS-6xHis-SUMO-dSynCas.v12-term-KanR | Protein | 208597 |
| | | purification | |

TABLE 5
Direct Repeat Sequence Table. Table listing direct repeat (DR)
nucleotide sequences. X-F/R and Y-F/R sequences are associated
with Cas7-1. Sequencescorrespond with DRs in FIGS. 6A-6C.

| | Enzyme | | |
|---|---|---|---|
| SEQ ID NO: | DR | RNA Sequence (5' -> 3') | Length |
| 75 | DRX | UUGUUUCAGUUCCUGCCGGCAGGAAUGUCCUU | 38 |
| | | UCAGAC | |
| 76 | DRY | GUCUGAAAGGACAUUCCUGCCGGCAGGAACUG | 38 |
| | | AAACAA | |
| 77 | DiCas7- | GGUUGgaaagccgguuuucuuugauGUCACGGAAC | 35 |
| | 11 | | |

TABLE 6
Additional Domain Patterns of Synthetic Proteins. The sequences
for these proteins are not in Table 2. Synthetic proteins are
representative of other iterations of domain combinations.

| | | | |
|---|---|---|---|
| Cas7-S1 | Cas7-S2 | Cas7-S4 | Cas7-S5 |
| Cas7.1: DiCas7-11 | Cas7.1: DiCas7-11 | Cas7.1: DiCas7-11 | Cas7.1: DiCas7-11 |
| L1: DiCas7-11 | L2: DiCas7-11 | L1: DiCas7-11 | L1: DiCas7-11 |
| Cas7.2: Cas7-1 | Cas7.2: Cas7-1 | Cas7.2: DiCas7-11 | L2: DiCas7-11 |
| and DiCas7-11 | and DiCas7-11 | L3: DiCas7-11 | Cas7.2: DiCas7-11 |
| L3: Cas7-1 | L3: Cas7-1 | Cas7.3: Cas7-1 | L3: DiCas7-11 |
| Cas7.3: Cas7-1 | Cas7.3: Cas7-1 | L4: Cas7-1 | Cas7.3: Cas7-1 |
| L4: Cas7-1 | L4: Cas7-1 | Cas7.4: Cas7-1 | L4: Cas7-1 |
| Cas7.4: Cas7-1 | Cas7.4: Cas7-1 | INS: Cas7-1 | Cas7.4: Cas7-1 |
| INS: Cas7-1 | INS: Cas7-1 | CTE: Cas7-1 | INS: Cas7-1 |
| CTE: Cas7-1 | CTE: Cas7-1 | Cas1: Cas7-1 | CTE: Cas7-1 |
| Cas1: Cas7-1 | Cas1: Cas7-1 | | Cas1: Cas7-1 |
| Cas7-6S | Cas7-8S | Cas7-S9 | Cas7-S16 |
| Cas7.1: DiCas7-11 | Cas7.1: DiCas7-11 | Cas7.1: DiCas7-11 | Cas7.1: DiCas7-11 |
| L1: DiCas7-11 | L1: DiCas7-11 | L1: DiCas7-11 | L1: DiCas7-11 |
| L2: DiCas7-11 | L2: DiCas7-11 | L2: DiCas7-11 | Cas1: Cas7-1 |
| Cas7.2: DiCas7-11 | Cas7.2: DiCas7-11 | Cas7.2: DiCas7-11 | L2: DiCas7-11 |
| L3: DiCas7-11 | L3: DiCas7-11 | L3: DiCas7-11 | Cas7.2: DiCas7-11 |
| Cas7.3: DiCas7-11 | Cas7.3: DiCas7-11 | Cas7.3: DiCas7-11 | L3: DiCas7-11 |
| L4: DiCas7-11 | L4: DiCas7-11 | L4: DiCas7-11 | Cas7.3: DiCas7-11 |
| Cas7.4: Cas7-1 | Cas7.4: DiCas7-11 | Cas7.4: DiCas7-11 | L4: DiCas7-11 |
| INS: Cas7-1 | INS: Cas7-1 | INS: Cas7-1 | Cas7.4: Cas7-1 |
| CTE: Cas7-1 | CTE: Cas7-1 | CTE: DiCas7-11 | INS: Cas7-1 |
| Cas1: Cas7-1 | Cas1: Cas7-1 | Cas1: Cas7-1 | CTE: Cas7-1 |
| Cas7-S17 | Cas7S-18 | Cas7-S19 | Cas7-S20 |
| Cas7.1: HvsCas7- | Cas7.1: HvsCas7- | Cas7.1: HvsCas7- | Cas7.1: HvsCas7- |
| 11 | 11 | 11 | 11 |
| L1: HvsCas7-11 | L1: HvsCas7-11 | L1: HvsCas7-11 | L1: HvsCas7-11 |
| Cas11: DiCas7-11 | Cas11: DiCas7-11 | Cas11: DiCas7-11 | Cas11: DiCas7-11 |
| L2: DiCas7-11 | L2: DiCas7-11 | L2: DiCas7-11 | L2: DiCas7-11 |
| Cas7.2: HvsCas7- | Cas7.2: HvsCas7- | Cas7.2: HvsCas7- | Cas7.2: DiCas7-11 |
| 11 | 11 | 11 | L3: DiCas7-11 |
| L3: DiCas7-11 | L3: DiCas7-11 | L3: DiCas7-11 | Cas7.3: HvsCas7- |
| Cas7.3: HvsCas7- | Cas7.3: Cas7-1 | Cas7.3: DiCas7-11 | 11 |
| 11 | L4: DiCas7-11 | L4: DiCas7-11 | L4: DiCas7-11 |
| L4: DiCas7-11 | Cas7.4: Cas7-1 | Cas7.4: Cas7-1 | Cas7.4: Cas7-1 |
| Cas7.4: Cas7-1 | INS: Cas7-1 | INS: Cas7-1 | INS: Cas7-1 |
| INS: Cas7-1 | CTE: HvsCas7-11 | CTE: HvsCas7-11 | CTE: HvsCas7-11 |
| CTE: HvsCas7-11 | | | |
| Cas7-S21 | Cas7-S22 | Cas7-S23 | Cas7-S24 |
| Cas7.1: HvsCas7- | Cas7.1: HvsCas7- | Cas7.1: HvsCas7- | Cas7.1: HvsCas7- |
| 11 | 11 | 11 | 11 |
| L1: HvsCas7-11 | L1: HvsCas7-11 | L1: HvsCas7-11 | L1: HvsCas7-11 |
| Cas11: DiCas7-11 | Cas11: DiCas7-11 | Cas11: HvsCas7- | Cas11: HvsCas7- |
| L2: DiCas7-11 | L2: DiCas7-11 | 11 | 11 |
| Cas7.2: DiCas7-11 | Cas7.2: DiCas7-11 | L2: DiCas7-11 | L2: DiCas7-11 |
| L3: DiCas7-11 | L3: DiCas7-11 | Cas7.2: HvsCas7- | Cas7.2: HvsCas7- |
| Cas7.3: Cas7-1 | Cas7.3: DiCas7-11 | 11 | 11 |
| L4: DiCas7-11 | L4: DiCas7-11 | L3: DiCas7-11 | L3: DiCas7-11 |
| Cas7.4: Cas7-1 | Cas7.4: Cas7-1 | Cas7.3: HvsCas7- | Cas7.3: Cas7-1 |
| INS: Cas7-1 | INS: Cas7-1 | 11 | L4: DiCas7-11 |
| CTE: HvsCas7-11 | CTE: HvsCas7-11 | L4: DiCas7-11 | Cas7.4: Cas7-1 |
| | | Cas7.4: Cas7-1 | INS: Cas7-1 |
| | | INS: Cas7-1 | CTE: HvsCas7-11 |
| | | CTE: HvsCas7-11 | |
| Cas7S-25 | Cas7S-26 | Cas7-S27 | Cas7-S28 |
| Cas7.1: HvsCas7- | Cas7.1: HvsCas7- | Cas7.1: HvsCas7- | Cas7.1: HvsCas7- |
| 11 | 11 | 11 | 11 |
| L1: HvsCas7-11 | L1: HvsCas7-11 | L1: HvsCas7-11 | L1: HvsCas7-11 |
| Cas11: HvsCas7- | Cas11: HvsCas7- | Cas11: HvsCas7- | Cas11: HvsCas7- |
| 11 | 11 | 11 | 11 |
| L2: DiCas7-11 | L2: DiCas7-11 | L2: DiCas7-11 | L2: DiCas7-11 |
| Cas7.2: HvsCas7- | Cas7.2: DiCas7-11 | Cas7.2: DiCas7-11 | Cas7.2: DiCas7-11 |
| 11 | L3: DiCas7-11 | L3: DiCas7-11 | L3: DiCas7-11 |
| L3: DiCas7-11 | Cas7.3: HvsCas7- | Cas7.3: Cas7-1 | Cas7.3: DiCas7-11 |
| Cas7.3: DiCas7-11 | 11 | L4: DiCas7-11 | L4: DiCas7-11 |
| L4: DiCas7-11 | L4: DiCas7-11 | Cas7.4: Cas7-1 | Cas7.4: Cas7-1 |
| Cas7.4: Cas7-1 | Cas7.4: Cas7-1 | INS: Cas7-1 | INS: Cas7-1 |
| INS: Cas7-1 | INS: Cas7-1 | CTE: HvsCas7-11 | CTE: HvsCas7-11 |
| CTE: HvsCas7-11 | CTE: HvsCas7-11 | | |
| Cas7S-29 | Cas7S-30 | Cas7-S31 | Cas7-S32 |
| Cas7.1: HvsCas7- | Cas7.1: HvsCas7- | Cas7.1: HvsCas7- | Cas7.1: HvsCas7- |
| 11 | 11 | 11 | 11 |
| L1: HvsCas7-11 | L1: HvsCas7-11 | L1: HvsCas7-11 | L1: HvsCas7-11 |
| Cas1: Cas7-1 | Cas1: Cas7-1 | Cas1: Cas7-1 | Cas1: Cas7-1 |
| L2: DiCas7-11 | L2: DiCas7-11 | L2: DiCas7-11 | L2: DiCas7-11 |
| Cas7.2: HvsCas7- | Cas7.2: HvsCas7- | Cas7.2: HvsCas7- | Cas7.2: DiCas7- |
| 11 | 11 | 11 | 11 |
| L3: DiCas7-11 | L3: DiCas7-11 | L3: DiCas7-11 | L3: DiCas7-11 |
| Cas7.3: HvsCas7- | Cas7.3: Cas7-1 | Cas7.3: DiCas7-11 | Cas7.3: HvsCas7-11 |
| 11 | L4: DiCas7-11 | L4: DiCas7-11 | L4: DiCas7-11 |
| L4: DiCas7-11 | Cas7.4: Cas7-1 | Cas7.4: Cas7-1 | Cas7.4: Cas7-1 |
| Cas7.4: Cas7-1 | INS: Cas7-1 | INS: Cas7-1 | INS: Cas7-1 |
| INS: Cas7-1 | CTE: HvsCas7-11 | CTE: HvsCas7-11 | CTE: HvsCas7-11 |
| CTE: HvsCas7-11 | | | |
| Cas7-S33 | Cas7S-34 | | |
| Cas7.1: HvsCas7- | Cas7.1: HvsCas7- | | |
| 11 | 11 | | |
| L1: HvsCas7-11 | L1: HvsCas7-11 | | |
| Cas1: Cas7-1 | Cas1: Cas7-1 | | |
| L2: DiCas7-11 | L2: DiCas7-11 | | |
| Cas7.2: DiCas7-11 | Cas7.2: DiCas7-11 | | |
| L3: DiCas7-11 | L3: DiCas7-11 | | |
| Cas7.3: Cas7-1 | Cas7.3: DiCas7-11 | | |
| L4: DiCas7-11 | L4: DiCas7-11 | | |
| Cas7.4: Cas7-1 | Cas7.4: Cas7-1 | | |
| INS: Cas7-1 | INS: Cas7-1 | | |
| CTE: HvsCas7-11 | CTE: HvsCas7-11 | | |

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group. All publications, patent applications, Appendices, patents, and other references mentioned herein or attached hereto are expressly incorporated by reference in their entirety, to the same extent as if each were incorporated by reference individually. In case of conflict, the present specification, including definitions, will control.

Other embodiments are set forth within the following claims.

### REFERENCES

1. K. S. Makarova, Y. I. Wolf, J. Iranzo, S. A. Shmakov, O. S. Alkhnbashi, S. J. J. Brouns, E. Charpentier, D. Cheng, D. H. Haft, P. Horvath, S. Moineau, F. J. M. Mojica, D. Scott, S. A. Shah, V. Siksnys, M. P. Terns, Č. Venclovas, M. F. White, A. F. Yakunin, W. Yan, F. Zhang, R. A. Garrett, R. Backofen, J. van der Oost, R. Barrangou, E. V. Koonin, Evolutionary classification of CRISPR-Cas systems: a burst of class 2 and derived variants. Nat. Rev. Microbiol. 18, 67-83 (2020).
2. S. P. B. van Beljouw, A. C. Haagsma, A. Rodríguez-Molina, D. F. van den Berg, J. N. A. Vink, S. J. J. Brouns, The gRAMP CRISPR-Cas effector is an RNA endonuclease complexed with a caspase-like peptidase. Science. 373, 1349-1353 (2021).
3. A. Özcan, R. Krajeski, E. Ioannidi, B. Lee, A. Gardner, K. S. Makarova, E. V. Koonin, O. O. Abudayyeh, J. S. Gootenberg, Programmable RNA targeting with the single-protein CRISPR effector Cas7-11. Nature, 1-6 (2021).
4. K. Kato, W. Zhou, S. Okazaki, Y. Isayama, T. Nishizawa, J. S. Gootenberg, O. O. Abudayyeh, H. Nishimasu, Structure and engineering of the type III-E CRISPR-Cas7-11 effector complex. Cell. 0 (2022), doi: 10.1016/j.cell.2022.05.003.
5. H. N. Goswami, J. Rai, A. Das, H. Li, Molecular mechanism of active Cas7-11 in processing CRISPR RNA and interfering target RNA. Elife. 11 (2022), doi: 10.7554/eLife.81678.
6. C. Hu, S. P. B. van Beljouw, K. H. Nam, G. Schuler, F. Ding, Y. Cui, A. Rodríguez-Molina, A. C. Haagsma, M. Valk, M. Pabst, S. J. J. Brouns, A. Ke, Craspase is a CRISPR RNA-guided, RNA-activated protease. Science. 377, 1278-1285 (2022).
7. A. B. Buchman, D. J. Brogan, R. Sun, T. Yang, P. D. Hsu, O. S. Akbari, Programmable RNA Targeting Using CasRx in Flies. CRISPR J. 3, 164-176 (2020).
8. J. F. Bot, J. van der Oost, N. Geijsen, The double life of CRISPR-Cas13. Curr. Opin. Biotechnol. 78, 102789 (2022).
9. C. P. Kelley, M. C. Haerle, E. T. Wang, Negative autoregulation mitigates collateral RNase activity of repeat-targeting CRISPR-Cas13d in mammalian cells. Cell Rep. 40, 111226 (2022).
10. Q. Wang, X. Liu, J. Zhou, C. Yang, G. Wang, Y. Tan, Y. Wu, S. Zhang, K. Yi, C. Kang, The CRISPR-Cas13a Gene-Editing System Induces Collateral Cleavage of RNA in Glioma Cells. Adv. Sci. 6, 1901299 (2019).
11. K. Kato, S. Okazaki, C. Schmitt-Ulms, K. Jiang, W. Zhou, J. Ishikawa, Y. Isayama, S. Adachi, T. Nishizawa, K. S. Makarova, E. V. Koonin, O. O. Abudayyeh, J. S. Gootenberg, H. Nishimasu, RNA-triggered protein cleavage and cell growth arrest by the type III-E CRISPR nuclease-protease. Science. 378, 882-889 (2022).
12. B. Ekundayo, D. Torre, B. Beckert, S. Nazarov, A. Myasnikov, H. Stahlberg, D. Ni, Structural insights into the regulation of Cas7-11 by TPR-CHAT. Nat. Struct. Mol. Biol. 30, 135-139 (2023).
13. K. S. Makarova, Y. I. Wolf, O. S. Alkhnbashi, F. Costa, S. A. Shah, S. J. Saunders, R. Barrangou, S. J. J. Brouns, E. Charpentier, D. H. Haft, P. Horvath, S. Moineau, F. J. M. Mojica, R. M. Terns, M. P. Terns, M. F. White, A. F. Yakunin, R. A. Garrett, J. van der Oost, R. Backofen, E. V. Koonin, An updated evolutionary classification of CRISPR-Cas systems. Nat. Rev. Microbiol. 13, 722-736 (2015).
14. W. X. Yan, S. Chong, H. Zhang, K. S. Makarova, E. V. Koonin, D. R. Cheng, D. A. Scott, Cas13d Is a Compact RNA-Targeting Type VI CRISPR Effector Positively Modulated by a WYL-Domain-Containing Accessory Protein. Mol. Cell. 70, 327-339.e5 (2018).
15. S. Konermann, P. Lotfy, N. J. Brideau, J. Oki, M. N. Shokhirev, P. D. Hsu, Transcriptome Engineering with RNA-Targeting Type VI-D CRISPR Effectors. Cell. 173, 665-676.e14 (2018).
16. S. Silas, G. Mohr, D. J. Sidote, L. M. Markham, A. Sanchez-Amat, D. Bhaya, A. M. Lambowitz, A. Z. Fire, Direct CRISPR spacer acquisition from RNA by a natural reverse transcriptase-Cas1 fusion protein. Science. 351 (2016), doi: 10.1126/science.aad4234.
17. J. Jumper, R. Evans, A. Pritzel, T. Green, M. Figurnov, O. Ronneberger, K. Tunyasuvunakool, R. Bates, A. Žídek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
18. M. Varadi, S. Anyango, M. Deshpande, S. Nair, C. Natassia, G. Yordanova, D. Yuan, O. Stroe, G. Wood, A. Laydon, A. Žídek, T. Green, K. Tunyasuvunakool, S. Petersen, J. Jumper, E. Clancy, R. Green, A. Vora, M. Lutfi, M. Figurnov, A. Cowie, N. Hobbs, P. Kohli, G. Kleywegt, E. Birney, D. Hassabis, S. Velankar, AlphaFold Protein Structure Database: massively expanding the structural coverage of protein-sequence space with high-accuracy models. Nucleic Acids Res. 50, D439-D444 (2022).
19. M. A. Cianfrocco, M. Wong-Barnum, C. Youn, R. Wagner, A. Leschziner, “COSMIC2: A Science Gateway for Cryo-Electron Microscopy Structure Determination” in Proceedings of the Practice and Experience in Advanced Research Computing 2017 on Sustainability, Success and Impact (Association for Computing Machinery, New York, NY, USA, 2017; https://doi.org/10.1145/3093338.3093390), PEARC17, pp. 1-5.
20. Y. Zheng, J. Li, B. Wang, J. Han, Y. Hao, S. Wang, X. Ma, S. Yang, L. Ma, L. Yi, W. Peng, Endogenous Type I CRISPR-Cas: From Foreign DNA Defense to Prokaryotic Engineering. Front Bioeng Biotechnol. 8, 62 (2020).
21. D. J. Brogan, D. Chaverra-Rodriguez, C. P. Lin, A. L. Smidler, T. Yang, L. M. Alcantara, I. Antoshechkin, J. Liu, R. R. Raban, P. Belda-Ferre, R. Knight, E. A. Komives, O. S. Akbari, Development of a Rapid and Sensitive CasRx-Based Diagnostic Assay for SARS-COV-2. ACS Sens (2021), doi: 10.1021/acssensors. 1c01088.
22. A. East-Seletsky, M. R. O'Connell, S. C. Knight, D. Burstein, J. H. D. Cate, R. Tjian, J. A. Doudna, Two distinct RNase activities of CRISPR-C2c2 enable guide-RNA processing and RNA detection. Nature. 538, 270-273 (2016).
23. B. Zetsche, J. S. Gootenberg, O. O. Abudayyeh, I. M. Slaymaker, K. S. Makarova, P. Essletzbichler, S. E. Volz, J. Joung, J. van der Oost, A. Regev, E. V. Koonin, F. Zhang, Cpf1 is a single RNA-guided endonuclease of a class 2 CRISPR-Cas system. Cell. 163, 759-771 (2015).
24. K. K. Yang, Z. Wu, F. H. Arnold, Machine-learning-guided directed evolution for protein engineering. Nat. Methods. 16, 687-694 (2019).
25. I. S. Al'Abri, D. J. Haller, Z. Li, N. Crook, Inducible directed evolution of complex phenotypes in bacteria. Nucleic Acids Res. 50, e58 (2022).
26. F. Adeola, Normalization of Gene Expression by Quantitative RT-PCR in Human Cell Line: comparison of 12 Endogenous Reference Genes. Ethiop. J. Health Sci. 28, 741-748 (2018).

1. A synthetic protein comprising first Cas7 peptide and a Cas1 or a Cas11 peptide, wherein the first Cas7 peptide is Cas7.1, and wherein the synthetic protein has RNA-targeting function.

2. The synthetic protein of claim 1, wherein the Cas1 peptide or Cas11 peptide is C-terminal relative to the first Cas7 peptide or wherein the Cas1 peptide is derived from an uncharacterized Cas1 peptide.

3. (canceled)

4. The synthetic protein of claim 1, wherein the Cas11 peptide is derived from a Cas7-11 effector.

5. The synthetic protein of claim 4, wherein the Cas7-11 effector is selected from DiCas7-11 or HvsCas7-11, optionally wherein the DiCas7-11 has a sequence according to SEQ ID NO: 6 and further optionally the HvsCas7-11 has a sequence according to SEQ ID NO: 14.

6. The synthetic protein of claim 1, wherein the Cas1 peptide is derived from a Cas7-1 effector, optionally wherein the Cas7-1 effector has a sequence according to SEQ ID NO: 1.

7. (canceled)

8. The synthetic protein of claim 1, further comprising a second, a third, and a fourth Cas7 peptide, and optionally wherein the second Cas7 peptide is Cas7.2, the third Cas7 peptide is Cas7.3, and the fourth Cas7 peptide is 7.4.

9. The synthetic protein of claim 8, wherein the second, third, and fourth Cas7 peptides are C-terminal relative to the first Cas7 peptide and the Cas1 or Cas11 peptide.

10. The synthetic protein of claim 1, wherein the first, second, third, and fourth Cas7 peptides are derived from at least one Cas7-1 effector and at least one Cas7-11 effector, optionally wherein the at least one Cas7-11 effector is selected from DiCas7-11, HvsCas7-11, OmCas7-11, or CsbCas7-11, optionally wherein the DiCas7-11 has a sequence according to SEQ ID NO: 6, optionally wherein the HvsCas7-11 has a sequence according to SEQ ID NO: 14, optionally wherein the OmCas7-11 has a sequence according to SEQ ID NO: 15, and further optionally wherein CsbCas7-11 has a sequence according to SEQ ID NO: 5.

11. The synthetic protein of claim 1, further comprising a first linker, optionally wherein the linker is located between the first Cas7 peptide and the Cas1 peptide or the Cas11 peptide.

12. The synthetic protein of claim 8, further comprising:
a second linker, a third linker, and a fourth linker,
wherein the linker is derived from at least one Cas7-11 or Cas7-1 effector, optionally wherein the first, second, third, and fourth linkers are the same or different, optionally wherein the second linker is located between the Cas1 peptide or the Cas11 peptide and the second Cas7 peptide, optionally wherein the third linker is located between the second Cas7 peptide and the third Cas7 peptide, and optionally wherein the fourth linker is located between the third Cas7 peptide and the fourth Cas7 peptide.

13-14. (canceled)

15. A vector comprising the synthetic protein of claim 1, optionally wherein the vector is, comprises, or is derived from a plasmid, an adenovirus, an adeno-associated virus (AAV), a retrovirus, a herpes simplex virus, a human immunodeficiency virus (HIV), or a synthetic vector.

16. A CRISPR system comprising the synthetic protein of claim 1, and a CRISPR RNA (crRNA).

17-18. (canceled)

19. The CRISPR system of claim 16, wherein the crRNA comprises an amino acid sequence comprising any one of SEQ ID NOs: 48, 49, 50, 51, 52, 53, 54, 55, 56, 57, 58, 59, 60, 61, 62, 63, 64, 65, 66, 67, 68, 69, 70, 71, 72, 73, or 74.

20. A composition comprising one or more of the synthetic protein of claim 1 and a carrier, optionally a pharmaceutically acceptable carrier.

21. (canceled)

22. A method to target and cleave an RNA molecule in vitro, comprising contacting a cell with the CRISPR system of claim 16 that targets an RNA sequence in the cell, optionally wherein the target sequence encodes a reporter gene or SARS-Cov-2 gene.

23. A method to target and cleave an RNA molecule in vivo, comprising contacting a cell with the CRISPR system of claim 16 that targets an RNA sequence in the cell, optionally wherein the target sequence encodes a reporter gene or SARS-Cov-2 gene.

24. A method to treat a microsatellite repeat expansion (MRE) disorder in a subject in need thereof, comprising administering to the subject an effective amount of the CRISPR system of any of claim 16 that targets an RNA sequence in the subject, wherein the RNA sequence encodes a gene associated with an MRE disorder, optionally wherein the subject in need is a mammal, and further optionally wherein the subject in need is a human.

25. (canceled)

26. A method to treat or prevent a viral disease in a subject in need thereof, comprising administering to the subject an effective amount of the CRISPR system of claim 16 that targets an RNA sequence.

27. A method to treat or prevent a disease in a plant, comprising administering to the plant an effective amount of the CRISPR system of claim 16 that targets an RNA sequence.

28-30. (canceled)
